(19) United States
(12) Patent Application Publication
(10) Pub. No.: US 2024/0350610 A1
(43) Pub. Date: Oct. 24, 2024

(54) RECOMBINANT CLASSICAL SWINE FEVER VIRUS E2 PROTEIN

(71) Applicant: Boehringer Ingelheim Vetmedica (China) Co., Ltd., Taizhou, Jiangsu (CN)

(72) Inventors: Ning Chen, Shanghai (CN); Huanhuan Liu, Shanghai (CN); Chao Tong, Shanghai (CN); Jiaying Wang, Shanghai (CN)

(21) Appl. No.: 18/032,530

(22) PCT Filed: Oct. 19, 2021
(86) PCT No.: PCT/CN2021/124794

§ 371 Date: Apr. 18, 2023

(30) Foreign Application Priority Data
Oct. 19, 2020 (WO) PCT/CN2020/121786

(51) Int. Cl. A61K 39/12; A61P 37/04; C07K 14/005

(52) U.S. Cl. CPC A61K 39/12; A61P 37/04; C07K 14/005; C12N 2770/24022; C12N 2770/24034; C12N 2770/24352

(57) ABSTRACT

The present invention relates to the field of animal health. Particularly, the present invention relates to a recombinant classical swine fever virus E2 protein comprising at least one mutation at the epitope specifically recognized by the 6B8 monoclonal antibody. Further, the present invention provides an immunogenic composition comprising the recombinant E2 protein of the present invention and the use of the immunogenic composition for preventing and/or treating diseases associated with CSFV in an animal. Moreover, the present invention provides a method and a kit for differentiating animals infected with CSFV from animals vaccinated with the immunogenic composition of the present invention. Furthermore, the present invention provides a method of producing the E2 protein.

### CROSS-REFERENCE TO RELATED APPLICATIONS

This application is a National Stage Application filed under 35 U.S.C. § 371 of International Patent Application No. PCT/CN2021/124794, filed on Oct. 19, 2021, which claims the benefit of International Patent Application No. PCT/CN2020/121786, filed on Oct. 19, 2020, the contents of each of which are hereby incorporated by reference in their entirety.

### TECHNICAL FIELD

The present invention relates to the field of animal health. Particularly, the present invention relates to a recombinant classical swine fever virus E2 protein comprising at least one mutation at the epitope specifically recognized by the 6B8 monoclonal antibody. Further, the present invention provides an immunogenic composition comprising the recombinant E2 protein of the present invention and the use of the immunogenic composition for preventing and/or treating diseases associated with CSFV in an animal. Moreover, the present invention provides a method and a kit for differentiating animals infected with CSFV from animals vaccinated with the immunogenic composition of the present invention. Furthermore, the present invention provides a method of producing the E2 protein.

### TECHNICAL BACKGROUND

Classical swine fever (CSF) is a highly contagious disease of pigs and wild boars that causes significant economic losses. The causative agent of the disease is classical swine fever virus (CSFV). In China, a combination of prophylactic vaccination and stamping out strategy is implemented to control CSF outbreaks. However, sporadic CSF outbreaks and persistent infection are still reported in most parts of China.

There is still a need in the art for a new CSFV vaccine that is safe, effective and animals vaccinated by which can be differentiated from those infected by wild type field strains.

### BRIEF DESCRIPTION OF THE INVENTION

In one aspect, the present invention provides a recombinant CSFV (classical swine fever virus) E2 protein comprising at least one mutation within the 6B8 epitope of the E2 protein, wherein the (unmodified) 6B8 epitope is specifically recognized by the 6B8 monoclonal antibody.

In one aspect, the present invention provides an isolate nucleic acid coding for the recombinant CSFV E2 protein of the present invention.

In one aspect, the present invention provides a vector comprising the nucleic acid of the present invention.

In one aspect, the present invention provides an immunogenic composition comprising the recombinant CSFV E2 protein, the nucleic acid encoding for the recombinant CSFV E2 protein, or the vector coding for such nucleic acid, each according to the present invention.

In one aspect, the present invention provides a method of preventing and/or treating diseases associated with CSFV in an animal, the method comprising the step of administering the immunogenic composition of the present invention to an animal in need thereof.

In one aspect, the present invention provides a method of differentiating animals infected with CSFV from animals vaccinated with the immunogenic composition of the present invention, comprising a) obtaining a sample from an animal; and b) analyzing said sample in an immuno test.

In one aspect, the present invention provides a kit for differentiating animals infected with CSFV from animals vaccinated with the immunogenic composition of the present invention.

### BRIEF DESCRIPTION OF THE DRAWINGS

FIG. 1: CSFV E2 structure and critical amino acids for 6B8 epitope.

FIG. 2: Construction of wildtype CSFV E2 and mutated CSFV E2 with various substitutions in 6B8 epitope.

FIG. 3: mAb 6B8 recognizes most CSFV strains while has no reaction with BVDV viruses.

FIG. 4: Sequence alignment of CSFV isolates, BVDV strains and some other Pestiviruses.

FIG. 5: IFA results showing that amino acid residues at position 14, 22, or 24/25 are critical for mAb 6B8 binding (FIGS. 5A-5C).

FIG. 6: A)-Purification results of wt-E2 and E2-KARD or E2-KRD, confirmed by both SDS PAGE and Western blotting (FIG. 6A); and B)-Purified E2-KARD or E2-KRD showed negative results with 6B8 staining (FIG. 6B).

FIG. 7: Purification results of QZ07-E2-Fc-WT, confirmed by both SDS PAGE and Western blotting.

FIG. 8: Post-challenge body temperature in the efficacy study.

FIG. 9: Post-challenge leucocyte counting in the efficacy study.

FIG. 10: Post-challenge mortality in the efficacy study.

FIG. 11: Post-challenge clinical score in the efficacy study.

FIG. 12: Serological response during the efficacy study.

FIG. 13: High yield of E2 protein expressed in CHO system.

FIG. 14: Immunogenicity of E2 protein expressed in CHO system.

FIG. 15: Identification of additional critical residues for 6B8 binding in E2. IFA detection of mutation forms of E2 protein in CHO cell expression system shows that amino acid residues at position 10, 41, or 64 are critical for mAb 6B8 binding.

FIG. 16: Western blot detection of mutation forms of E2 protein in CHO cell expression system.

FIG. 17: Identification of additional amino acids at position 22 for inhibiting 6B8 binding in E2.

FIG. 18: Identification of additional amino acids at position 24 for inhibiting 6B8 binding in E2.

FIG. 19: Identification of additional amino acids at position 25 for inhibiting 6B8 binding in E2.

FIG. 20: Identification of additional amino acids at position 64 for inhibiting 6B8 binding in E2.

### DETAILED DESCRIPTION

Before the aspects of the present invention are described, it must be noted that as used herein and in the appended claims, the singular forms “a”, “an”, and “the” include plural reference unless the context clearly dictates otherwise. Thus, for example, reference to “a or an epitope” includes a plurality of epitopes, reference to the “virus” is a reference to one or more viruses and equivalents thereof known to those skilled in the art, and so forth. The term “and/or” is intended to encompass any combinations of the items connected by this term, equivalent to listing all the combinations individually. For example. “A, B and/or C” encompasses “A”, “B”, “C”, “A and B”. “A and C”, “B and C”, and “A and B and C”. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the virus strains, the cell lines, vectors, and methodologies as reported in the publications which might be used in connection with the invention. Nothing herein is to be construed as an admission that the invention is not entitled to antedate such disclosure by virtue of prior invention.

In one aspect, the present invention provides a recombinant CSFV (classical swine fever virus) E2 protein comprising at least one mutation within the 6B8 epitope of the E2 protein, wherein the (unmodified) 6B8 epitope is specifically recognized by the 6B8 monoclonal antibody.

The term “CSFV” as used herein refers to all viruses belonging to species of classical swine fever virus (CSFV) in the genus Pestivirus within the family Flaviviridae.

The term “recombinant” refers to a protein or a nucleic acid that has been altered, rearranged, or modified by genetic engineering. However, the term does not refer to alterations in polynucleotide, amino acid sequence, nucleotide sequence that result from naturally occurring events, such as spontaneous mutations.

In one aspect, the recombinant CSFV E2 protein is isolated.

A polypeptide or nucleic acid molecule is considered to be “isolated”—for example, when compared to its native biological source and/or the reaction medium or cultivation medium from which it has been obtained—when it has been separated from at least one other component with which it is usually associated in said source or medium, such as another protein/polypeptide, another nucleic acid, another biological component or macromolecule or at least one contaminant, impurity or minor component. In particular, a polypeptide or nucleic acid molecule is considered “isolated” when it has been purified at least 2-fold, in particular at least 10-fold, more in particular at least 100-fold, and up to 1000-fold or more. A polypeptide or nucleic acid molecule that is “in isolated form” is preferably essentially homogeneous, as determined using a suitable technique, such as a suitable chromatographical technique, such as polyacrylamide gel electrophoresis.

“The 6B8 epitope of the E2 protein” herein also refers to an epitope of the E2 protein specifically recognized by the 6B8 monoclonal antibody as disclosed herein. The 6B8 epitope may be a conformational epitope. The 6B8 epitope may comprise defined amino acids at positions 24, 25, 14, 22, 10, 41 and/or 64 of the E2 protein. For example, the 6B8 epitope may comprise S at position 14, G at position 22, E or G at position 24, G at position 25, Y at position 10, D at position 41, and/or R at position 64 of the E2 protein.

The term “6B8 monoclonal antibody” refers to the 6B8 monoclonal antibody or an antigen-binding fragment thereof, wherein the 6B8 monoclonal antibody specifically recognizes the 6B8 epitope, in particular the 6B8 epitope that comprises at least the amino acid S at position 14, G at position 22, E or G at position 24, G at position 25, Y at position 10, D at position 41, and/or R at position 64 of the E2 protein. Preferably, the term 6B8 monoclonal antibody refers to a monoclonal antibody that comprises CDRs of the monoclonal antibody produced by a hybridoma deposited at CCTCC under the accession number CCTCC C2018120. Preferably, the term 6B8 monoclonal antibody refers to a monoclonal antibody that comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6. More preferably, the term 6B8 monoclonal antibody refers to a monoclonal antibody that comprises a heavy chain variable region (V_{H}) having an amino acid sequence as set forth in SEQ ID NO: 7 and a light chain variable region (V_{L}) having an amino acid sequence as set forth in SEQ ID NO: 8. More preferably the term 6B8 monoclonal antibody refers to the monoclonal antibody produced by a hybridoma deposited at CCTCC under the accession number CCTCC C2018120.

As used herein. “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Hence, an antibody includes any protein having a binding domain that is homologous or substantially homologous to an immunoglobulin antigen-binding domain (antibody combining site). Antibodies include antibody fragments. As used herein, the term antibody, thus, includes synthetic antibodies, recombinantly produced antibodies, multispecific antibodies (e.g., bispecific antibodies), human antibodies, non-human antibodies, humanized antibodies, chimeric antibodies, intrabodies, and antibody fragments. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

The term “variable region” as used herein means an immunoglobulin domain essentially consisting of four “framework regions” which are referred to in the art and hereinbelow as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and hereinbelow as “complementarity determining region 1” or “CDR1”; as “complementarity determining region 2” or “CDR2”; and as “complementarity determining region 3” or “CDR3”, respectively. Thus, the general structure or sequence of an immunoglobulin variable region can be indicated as follows: FR1-CDR1-FR2-CDR2-FR3-CDR3-FR4. VH or V_{H} refers to a heavy chain variable region, and VL or V_{L} refers to a light chain variable region. Similarly, VH CDR1, VH CDR2 and VH CDR3 refer to CDR1, CDR2 and CDR3 of a heavy chain variable region, respectively, VL CDR1, VL CDR2 and VL CDR3 refer to CDR1, CDR2 and CDR3 of a light chain variable region, respectively.

As used herein, an “antibody fragment” or “antigen-binding fragment” of an antibody refers to any portion of a full-length antibody that is less than full length but contains at least a portion of the variable region of the antibody that binds antigen (e.g., one or more CDRs and/or one or more antibody combining sites) and thus retains the binding specificity, and at least a portion of the specific binding ability of the full-length antibody. Hence, an antigen-binding fragment refers to an antibody fragment that contains an antigen-binding portion that binds to the same antigen as the antibody from which the antibody fragment is derived. Antibody fragments include antibody derivatives produced by enzymatic treatment of full-length antibodies, as well as synthetically. e.g. recombinantly produced derivatives. An antibody fragment is included among antibodies. Examples of antibody fragments include, but are not limited to, Fab, Fab′, F(ab′) 2, single-chain Fv (scFv), Fv, dsFv, diabody, Fd and Fd′ fragments and other fragments, including modified fragments (sec. for example. Methods in Molecular Biology, Vol 207: Recombinant Antibodies for Cancer Therapy Methods and Protocols (2003); Chapter 1; p 3-25. Kipriyanov). The fragment can include multiple chains linked together, such as by disulfide bridges and/or by peptide linkers. An antigen-binding fragment includes any antibody fragment that when inserted into an antibody framework (such as by replacing a corresponding region) results in an antibody that immunospecifically binds (i.e. exhibits Ka of at least or at least about 10⁷-10⁸ M⁻¹) to the antigen.

The term “antigen-binding fragment of the 6B8 monoclonal antibody” refers to a fragment of the 6B8 monoclonal antibody or at least encodes for an amino acid sequence that specifically recognizes the 6B8 epitope, in particular the 6B8 epitope that comprises at least the amino acid S at position 14, G at position 22, E or G at position 24, G at position 25, Y at position 10, D at position 41, and/or R at position 64 of the E2 protein. The term further encompasses an amino acid fragment coding for a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO: 3, and/or a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6. Moreover, the term also encompasses an amino acid fragment that comprises a heavy chain variable region (V_{H}) having an amino acid sequence as set forth in SEQ ID NO: 7 and/or a light chain variable region (V_{L}) having an amino acid sequence as set forth in SEQ ID NO: 8. More preferably the term encompasses an amino acid fragment encoded by the monoclonal antibody produced by a hybridoma deposited at CCTCC under the accession number CCTCC C2018120, which amino acid fragment specifically binds to the 6B8 epitope.

The term “mutation” includes substitution, deletion or addition of one or more amino acids. The term mutation is well known to the person skilled in the art and the person skilled in the art can generate mutations without further ado.

In one aspect, the at least one mutation within the 6B8 epitope of the E2 protein of the invention leads to a specific inhibition of the binding of 6B8 monoclonal antibody to such mutated 6B8 epitope.

The term “specifically inhibits or specific inhibition” means that the 6B8 antibody binds with an at least 2-times, preferably 5-times, more preferably 10-times and even more preferably 50-times lower affinity to the mutated 6B8 epitope in comparison to the unmodified 6B8 epitope, in particular to the unmodified 6B8 epitope having the amino acid S at position 14, G at position 22, E or G at position 24, G at position 25, Y at position 10, D at position 41, R at position 64 of the E2 protein, or combinations thereof. “Affinity” is the interaction between a single antigen-binding site on an antibody molecule and a single epitope. It is expressed by the association constant KA=kass/kdiss, or the dissociation constant KD=K_{diss}/kₐₛₛ. More preferably, the term “specifically inhibits or specific inhibition” means that the 6B8 monoclonal antibody, in particular the monoclonal antibody produced by a hybridoma deposited at CCTCC under the accession number CCTCC C2018120 does not detectably bind to the mutated 6B8 epitope according the invention in an specific immunofluorescence assay, preferably in the specific immunofluorescence assay as described in example 5, or in a specific Dot blot assay, preferably in the specific Dot blot assay as described in example 6. Both the specific immunofluorescence assay and the specific Dot blot assay can be used to determine the specific inhibition, however, if conflict results are obtained from the two assays, the result from Dot blot assay prevails.

The term “substitution” means that an amino acid is replaced by another amino acid at the same position. Thus, the term substitution covers the removal/deletion of an amino acid, followed by insertion of another amino acid at the same position.

The term “E2 protein” refers to the processed E2 protein which results as final cleavage product from the polyprotein (Npro-C-Erns-E1-E2-p7-NS2-NS3-NS4A-NS4B-NS5A-NS5B) of the CSFV. A person skilled in the art would acknowledge that any E2 protein of CSFV can be used in the invention. In one aspect of the invention, the recombinant E2 protein is derived from a wildtype E2 protein having a 6B8 epitope specifically recognized by the 6B8 monoclonal antibody. For example, the E2 protein can be derived from a known CSFV strain such as C-strain, or from new isolates, such as QZ07 or GD18 as defined herein. For example, the E2 protein of the field strain QZ07 has the amino acid sequence set forth in SEQ ID NO:9, the E2 protein of the field strain GD18 has the amino acid sequence set forth in SEQ ID NO:10, the E2 protein of the field strain GD191 has the amino acid sequence set forth in SEQ ID NO: 34, and the E2 protein of C-strain has the amino acid sequence set forth in SEQ ID NO:21.

In one aspect of the invention, the recombinant E2 protein comprises an amino acid sequence having at least 75%, at least 80%, at least 85%, at least 90%, at least 91%, at least 92%, at least 93%, at least 94%, at least 95%, at least 96%, at least 97%, at least 98%, at least 99% sequence identity to any one of SEQ ID NO: 9, 10, 34 and 21, but contains at least one mutation within the 6B8 epitope as disclosed herein.

“Sequence identity” between two polypeptide sequences indicates the percentage of amino acids that are identical between the sequences. Methods for evaluating the level of sequence identity between amino acid or nucleotide sequences are known in the art. For example, sequence analysis softwares are often used to determine the identity of amino acid sequences. For example, identity can be determined by using the BLAST program at NCBI database. For determination of sequence identity, see e.g., Computational Molecular Biology, Lesk. A. M., ed., Oxford University Press, New York, 1988; Biocomputing: Informatics and Genome Projects. Smith, D. W., ed., Academic Press, New York, 1993; Computer Analysis of Sequence Data, Part I, Griffin. A. M., and Griffin, H. G., eds., Humana Press, New Jersey. 1994; Sequence Analysis in Molecular Biology, von Heinje, G., Academic Press. 1987 and Sequence Analysis Primer, Gribskov. M, and Devereux, J., eds., M Stockton Press, New York, 1991.

In a preferred aspect of the invention, the recombinant E2 protein having at least one mutation within the 6B8 epitope as disclosed herein is immunogenic and preferably confers protective immunity against CSFV. The E2 protein contains four antigenic domains. A. B. C and D domain, and all these domains are located at the N-terminal of the E2 protein. The four domains constitute two independent antigenic units, one is the unit of B/C domains and the other comprises A/D domains. The B/C domain is from amino acid position 1 to positions 84/111 and D/A domain is located from amino acid position 77 to positions 111/177. Furthermore, the B/C domain is linked by a putative disulfide bond between amino acid 4C and 48C, while the unit D/A is formed with two disulfide bonds, one between amino acids 103C and 167C, and the other between amino acids 129C and 139C. Those Cysteine residues are crucial for conformation antigenic structure of E2 protein. Antigenic motif (82-85LLFD) are important for the antigenic structure of E2 protein for convalescent serum binding. Another motif (RYLASLHKKALPT, amino acid positions 64 to 76) is also identified important for the structural integrity of conformational epitope recognition of E2 protein. In addition it is reported that E2 protein containing merely one of above mentioned antigenic domain remained immunogenic and can protects pigs from infectious CSFV challenge. Therefore, in a preferred aspect of the invention, the recombinant E2 protein having at least one modification within the 6B8 epitope as described herein retains at least one, preferably at least one of the antigenic domains as described above. Preferably, the recombinant E2 protein of the invention can confer protective immunity against CSFV. In one aspect, the at least one mutation within the 6B8 epitope as defined herein can be introduced without substantially affects the protective immunogenicity of the recombinant E2 protein against CSFV.

In one aspect of the invention, the 6B8 epitope of the E2 protein specifically recognized by the 6B8 monoclonal antibody is defined at least by the amino acid residue at position 14, position 22, position 24, positions 24 and 25 (“24/25”), position 10, position 41 and/or position 64 of the E2 protein.

In one aspect of the invention, the 6B8 epitope of the E2 protein specifically recognized by the 6B8 monoclonal antibody is defined at least by the amino acid residue S14, G22, E24, E24/G25, Y10, D41 and/or R64 of the E2 protein, such as for isolates QZ07, GD18 or GD191. In one aspect of the invention, the 6B8 epitope of the E2 protein specifically recognized by the 6B8 monoclonal antibody is defined at least by the amino acid residue S14, G22, G24, G24/G25, Y10, D41 and/or R64 of the E2 protein, such as for C-strain.

The numbering of the amino acid residue refers to the amino acid position in the processed E2 protein from the N-terminal, e.g. to the amino acid position as provide in SEQ ID NO:9 or 10 in an exemplary manner. However, the amino acid position can further be defined in relation to the polyprotein (containing Npro-C-Erns-E1-E2-p7-NS2-NS3-NS4A-NS4B-NS5A-NS5B), e.g. to the amino acid position as provide in SEQ ID NO: 11 or 12 in an exemplary manner. For example, amino acid residues at position 14, position 22, position 24, position 25, position 10, position 41 and position 64 of the E2 protein corresponds to amino acid residues at position 703, position 711, position 713, position 714, position 699, position 730, and position 753 of the polyprotein.

In one aspect of the invention, the recombinant CSFV E2 protein according to the invention comprises a substitution at amino acid position 24 of the E2 protein, a substitution at amino acid positions 24/25 of the E2 protein, a substitution at amino acid position 14 of the E2 protein, a substitution at amino acid position 22 of the E2 protein, a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 10 of the E2 protein, a substitution at amino acid positions 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 24 of the E2 protein and a substitution at amino acid position 25 of the E2 protein, and at least one further substitution selected from the group consisting of a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 24 of the E2 protein and a substitution at amino acid position 14 of the E2 protein at least one further substitution selected from the group consisting of a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 24 of the E2 protein, a substitution at amino acid position 25 of the E2 protein and a substitution at amino acid position 14 of the E2 protein, and at least one further substitution selected from the group consisting of a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 24 of the E2 protein and a substitution at amino acid position 22 of the E2 protein, at least one further substitution selected from the group consisting of a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 24 of the E2 protein, a substitution at amino acid position 25 of the E2 protein and a substitution at amino acid position 22 of the E2 protein, and at least one further substitution selected from the group consisting of a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV according to the invention comprises a substitution at amino acid position 14 of the E2 protein and a substitution at amino acid position 22 of the E2 protein, and at least one further substitution selected from the group consisting of a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 24 of the E2 protein, a substitution at amino acid position 14 of the E2 protein, and a substitution at amino acid position 22 of the E2 protein, and at least one further substitution selected from the group consisting of a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 24 of the E2 protein, a substitution at amino acid position 25 of the E2 protein, a substitution at amino acid position 14 of the E2 protein, and a substitution at amino acid position 22 of the E2 protein, and at least one further substitution selected from the group consisting of a substitution at amino acid position 10 of the E2 protein, a substitution at amino acid position 41 of the E2 protein, and/or a substitution at amino acid position 64 of the E2 protein, with a substitution at amino acid position 64 being preferred.

In one aspect of the invention, the recombinant CSFV E2 protein according to the invention comprises a substitution at amino acid position 24 of the E2 protein, a substitution at amino acid position 25 of the E2 protein, a substitution at amino acid position 14 of the E2 protein, a substitution at amino acid position 22 of the E2 protein, and a substitution at amino acid position 64 of the E2 protein.

In one aspect of the invention, the recombinant CSFV E2 protein according to the invention comprises substitutions at the amino acid positions of the E2 protein as defined in the following tables 1 to 7. For example, the second line in table 1 means that the recombinant CSFV E2 protein according to the invention comprises a substitution at amino acid position 10 (“position 10”) of the E2 protein, and the third line in table 1 means that the recombinant CSFV E2 protein according to the invention comprises substitutions at amino acid positions 10 and 14 of the E2 protein, etc.

TABLE 1

| position | position | position | position | position | positions | position | position |
|---|---|---|---|---|---|---|---|
| 10 | 14 | 22 | 24 | 25 | 24/25 | 41 | 64 |
| x | | | | | | | |
| x | x | | | | | | |
| x | | x | | | | | |
| x | | | x | | | | |
| x | | | | x | x | | |
| x | | | | | | x | |
| x | | | | | | | x |
| x | x | x | | | | | |
| x | x | | x | | | | |
| x | x | | | x | x | | |
| x | x | | | | | x | |
| x | x | | | | | | x |
| x | | x | x | | | | |
| x | | x | | x | x | | |
| x | | x | | | | x | |
| x | | x | | | | | x |
| x | | | x | x | x | | |
| x | | | x | | | x | |
| x | | | x | | | | x |
| x | | | | x | x | x | |
| x | | | | x | x | | x |
| x | | | | | | x | x |
| x | x | x | x | | | | |
| x | x | x | | x | x | | |
| x | x | x | | | | x | |
| x | x | x | | | | | x |
| x | x | | x | x | x | | |
| x | x | | x | | | x | |
| x | x | | x | | | | x |
| x | x | | | x | x | x | |
| x | x | | | x | x | | x |
| x | x | | | | | x | x |
| x | | x | x | x | x | | |
| x | | x | x | | | x | |
| x | | x | x | | | | x |
| x | | | x | x | x | x | |
| x | | | x | x | x | | x |
| x | | | | x | x | x | x |
| x | x | x | x | x | x | | |
| x | x | x | x | | | x | |
| x | x | x | x | | | | x |
| x | | x | x | x | x | x | |
| x | | x | x | x | x | | |
| x | x | x | x | x | x | x | |
| x | x | x | x | x | x | | x |
| x | x | x | x | x | x | x | x |

TABLE 2

| position | position | position | position | position | position | positions | position |
|---|---|---|---|---|---|---|---|
| 41 | 10 | 14 | 22 | 24 | 25 | 24/25 | 64 |
| x | | | | | | | |
| x | x | | | | | | |
| x | | x | | | | | |
| x | | | x | | | | |
| x | | | | x | | | |
| x | | | | | x | x | |
| x | | | | | | | x |
| x | x | x | | | | | |
| x | x | | x | | | | |
| x | x | | | x | | | |
| x | x | | | | x | x | |
| x | x | | | | | | x |
| x | | x | x | | | | |
| x | | x | | x | | | |
| x | | x | | | x | x | |
| x | | x | | | | | x |
| x | | | x | x | | | |
| x | | | x | | x | x | |
| x | | | x | | | | x |
| x | | | | x | x | x | |
| x | | | | x | | | x |
| x | | | | | x | x | x |
| x | x | x | x | | | | |
| x | x | x | | x | | | |
| x | x | x | | | x | x | |
| x | x | x | | | | | x |
| x | x | | x | x | | | |
| x | x | | x | | x | x | |
| x | x | | x | | | | x |
| x | x | | | x | x | x | |
| x | x | | | x | | | x |
| x | x | | | | x | x | x |
| x | | x | x | x | | | |
| x | | x | x | | x | x | |
| x | | x | x | | | | x |
| x | | | x | x | x | x | |
| x | | | x | x | | | x |
| x | | | | x | x | x | x |
| x | x | x | x | x | | | |
| x | x | x | x | | x | x | |
| x | x | x | x | | | | x |
| x | | x | x | x | x | x | |
| x | | x | x | x | | | x |
| x | x | x | x | x | x | x | |
| x | x | x | x | x | | | x |
| x | x | x | x | x | x | x | x |

TABLE 3

| position | position | position | position | position | position | positions | position |
|---|---|---|---|---|---|---|---|
| 64 | 10 | 14 | 22 | 24 | 25 | 24/25 | 41 |
| x | | | | | | | |
| x | x | | | | | | |
| x | | x | | | | | |
| x | | | x | | | | |
| x | | | | x | | | |
| x | | | | | x | x | |
| x | | | | | | | x |
| x | x | x | | | | | |
| x | x | | x | | | | |
| x | x | | | x | | | |
| x | x | | | | x | x | |
| x | x | | | | | | x |
| x | | x | x | | | | |
| x | | x | | x | | | |
| x | | x | | | x | x | |
| x | | x | | | | | x |
| x | | | x | x | | | |
| x | | | x | | x | x | |
| x | | | x | | | | x |
| x | | | | x | x | x | |
| x | | | | x | | | x |
| x | | | | | x | x | x |
| x | x | x | x | | | | |
| x | x | x | | x | | | |
| x | x | x | | | x | x | |
| x | x | x | | | | | x |
| x | | | x | x | | | |
| x | x | | x | | x | x | |
| x | x | | x | | | | x |
| x | x | | | x | x | x | |
| x | x | | | x | | | x |
| x | x | | | x | x | x | |
| x | x | | | x | | | x |
| x | x | | | | x | x | x |
| x | | x | x | x | | | |
| x | | x | x | | x | x | |
| x | | x | x | | | | x |
| x | | | x | x | x | x | |
| x | | | x | x | | | x |
| x | | | | x | x | x | x |
| x | x | x | x | x | | | |
| x | x | x | x | | x | x | |
| x | x | x | x | | | | x |
| x | | x | x | x | x | x | |
| x | | x | x | x | | | x |
| x | x | x | x | x | x | x | |
| x | x | x | x | x | | | x |
| x | x | x | x | x | x | x | x |

TABLE 4

| position | position | position | position | position | positions | position | position |
|---|---|---|---|---|---|---|---|
| 14 | 10 | 22 | 24 | 25 | 24/25 | 41 | 64 |
| x | | | | | | | |
| x | x | | | | | | |
| x | | x | | | | | |
| x | | | x | | | | |
| x | | | | x | x | | |
| x | | | | | | x | |
| x | | | | | | | x |
| x | x | x | | | | | |
| x | x | | x | | | | |
| x | x | | | x | x | | |
| x | x | | | | | x | |
| x | x | | | | | | x |
| x | | x | x | | | | |
| x | | x | | x | x | | |
| x | | x | | | | x | |
| x | | x | | | | | x |
| x | | | x | x | x | | |
| x | | | x | | | x | |
| x | | | x | | | | x |
| x | | | | x | x | x | |
| x | | | | x | x | | x |
| x | | | | | | x | x |
| x | x | x | x | | | | |
| x | x | x | | x | x | | |
| x | x | x | | | | x | |
| x | x | x | | | | | x |
| x | x | | x | x | x | | |
| x | x | | x | | | x | |
| x | x | | x | | | | x |
| x | x | | | x | x | x | |
| x | x | | | x | x | | x |
| x | x | | | | | x | x |
| x | | x | x | x | x | | |
| x | | x | x | | | x | |
| x | | x | x | | | | x |
| x | | | x | x | x | x | |
| x | | | x | x | x | | x |
| x | | | | x | x | x | x |
| x | x | x | x | x | x | | |
| x | x | x | x | | | x | |
| x | x | x | x | | | | x |
| x | | x | x | x | x | x | |
| x | | x | x | x | x | | x |
| x | x | x | x | x | x | x | |
| x | x | x | x | x | x | | x |
| x | x | x | x | x | x | x | x |

TABLE 5

| position | position | position | position | | positions | position | position |
|---|---|---|---|---|---|---|---|
| 22 | 10 | 14 | 24 | position25 | 24/25 | 41 | 64 |
| x | | | | | | | |
| x | x | | | | | | |
| x | | x | | | | | |
| x | | | x | | | | |
| x | | | | x | x | | |
| x | | | | | | x | |
| x | | | | | | | x |
| x | x | x | | | | | |
| x | x | | x | | | | |
| x | x | | | x | x | | |
| x | x | | | | | x | |
| x | x | | | | | | x |
| x | | x | x | | | | |
| x | | x | | x | x | | |
| x | | x | | | | x | |
| x | | x | | | | | x |
| x | | | x | x | x | | |
| x | | | x | | | x | |
| x | | | x | | | | x |
| x | | | | x | x | x | |
| x | | | | x | x | | x |
| x | | | | | | x | x |
| x | x | x | x | | | | |
| x | x | x | | x | x | | |
| x | x | x | | | | x | |
| x | x | x | | | | | x |
| x | x | | x | x | x | | |
| x | x | | x | | | x | |
| x | x | | x | | | | x |
| x | x | | | x | x | x | |
| x | x | | | x | x | | x |
| x | x | | | | | x | x |
| x | | x | x | x | x | | |
| x | | x | x | | | x | |
| x | | x | x | | | | x |
| x | | | x | x | x | x | |
| x | | | x | x | x | | x |
| x | | | | x | x | x | x |
| x | x | x | x | x | x | | |
| x | x | x | x | | | x | |
| x | x | x | x | | | | x |
| x | | x | x | x | x | x | |
| x | | x | x | x | x | | x |
| x | x | x | x | x | x | x | |
| x | x | x | x | x | x | | x |
| x | x | x | x | x | x | x | x |

TABLE 6

| position | position | position | position | position | positions | position | position |
|---|---|---|---|---|---|---|---|
| 24 | 10 | 14 | 22 | 25 | 24/25 | 41 | 64 |
| x | | | | | | | |
| x | x | | | | | | |
| x | | x | | | | | |
| x | | | x | | | | |
| x | | | | x | x | | |
| x | | | | | | x | |
| x | | | | | | | x |
| x | x | x | | | | | |
| x | x | | x | | | | |
| x | x | | | x | x | | |
| x | x | | | | | x | |
| x | x | | | | | | x |
| x | | x | x | | | | |
| x | | x | | x | x | | |
| x | | x | | | | x | |
| x | | x | | | | | x |
| x | | | x | x | x | | |
| x | | | x | | | x | |
| x | | | x | | | | x |
| x | | | | x | x | x | |
| x | | | | x | x | | x |
| x | | | | | | x | x |
| x | x | x | x | | | | |
| x | x | x | | x | x | | |
| x | x | x | | | | x | |
| x | x | x | | | | | x |
| x | x | | x | x | x | | |
| x | x | | x | | | x | |
| x | x | | x | | | | x |
| x | x | | | x | x | x | |
| x | x | | | x | x | | |
| x | x | | | | | x | x |
| x | | x | x | x | x | | |
| x | | x | x | | | x | |
| x | | x | x | | | | x |
| x | | | x | x | x | x | |
| x | | | x | x | x | | x |
| x | | | | x | x | x | x |
| x | x | x | x | x | x | | |
| x | x | x | x | | | x | |
| x | x | x | x | | | | x |
| x | | x | x | x | x | x | |
| x | | x | x | x | x | | x |
| x | x | x | x | x | x | x | |
| x | x | x | x | x | x | | x |
| x | x | x | x | x | x | x | x |

TABLE 7A

| position | position | position | position | position | position | position |
|---|---|---|---|---|---|---|
| 25 | 10 | 14 | 22 | 24 | 41 | 64 |
| x | | | | | | |
| x | x | | | | | |
| x | | x | | | | |
| x | | | x | | | |
| x | | | | x | | |
| x | | | | | x | |
| x | | | | | | x |
| x | x | x | | | | |
| x | x | | x | | | |
| x | x | | | x | | |
| x | x | | | | x | |
| x | x | | | | | x |
| x | | x | x | | | |
| x | | x | | x | | |
| x | | x | | | x | |
| x | | x | | | | x |
| x | | | x | x | | |
| x | | | x | | x | |
| x | | | x | | | x |
| x | | | | x | x | |
| x | | | | x | | x |
| x | | | | | x | x |
| x | x | x | x | | | |
| x | x | x | | x | | |
| x | x | x | | | x | |
| x | x | x | | | | x |
| x | x | | x | x | | |
| x | x | | x | | x | |
| x | x | | x | | | x |
| x | x | | | x | x | |
| x | x | | | x | | x |
| x | x | | | | x | x |
| x | | x | x | x | | |
| x | | x | x | | x | |
| x | | x | x | | | x |
| x | | | x | x | x | |
| x | | | x | x | | x |
| x | | | | x | x | x |
| x | x | x | x | x | | |
| x | x | x | x | | x | |
| x | x | x | x | | | x |
| x | | x | x | x | x | |
| x | | x | x | x | | x |
| x | x | x | x | x | x | |
| x | x | x | x | x | | x |
| x | x | x | x | x | x | x |

TABLE 7B

| positions | position | position | position | position | position | position |
|---|---|---|---|---|---|---|
| 24/25 | 10 | 14 | 22 | 24 | 41 | 64 |
| x | | | | | | |
| x | x | | | | | |
| x | | x | | | | |
| x | | | x | | | |
| x | | | | x | | |
| x | | | | | x | |
| x | | | | | | x |
| x | x | x | | | | |
| x | x | | x | | | |
| x | x | | | x | | |
| x | x | | | | x | |
| x | x | | | | | x |
| x | | x | x | | | |
| x | | x | | x | | |
| x | | x | | | x | |
| x | | x | | | | x |
| x | | | x | x | | |
| x | | | x | | x | |
| x | | | x | | | x |
| x | | | | x | x | |
| x | | | | x | | x |
| x | | | | | x | x |
| x | x | x | x | | | |
| x | x | x | | x | | |
| x | x | x | | | x | |
| x | x | x | | | | x |
| x | x | | x | x | | |
| x | x | | x | | x | |
| x | x | | x | | | x |
| x | x | | | x | x | |
| x | x | | | x | | x |
| x | x | | | | x | x |
| x | | x | x | x | | |
| x | | x | x | | x | |
| x | | x | x | | | x |
| x | | | x | x | x | |
| x | | | x | x | | x |
| x | | | | x | x | x |
| x | x | x | x | x | | |
| x | x | x | x | | x | |
| x | x | x | x | | | x |
| x | | x | x | x | x | |
| x | | x | x | x | | x |
| x | x | x | x | x | x | |
| x | x | x | x | x | | x |
| x | x | x | x | x | x | x |

In one aspect of the invention, in the recombinant CSFV E2 protein according to the invention the amino acid at position 10 of the E2 protein is substituted to A or P, the amino acid at position 14 of the E2 protein is substituted to K, Q or R, the amino acid at position 22 of the E2 protein is substituted to A, R, Q, E, D, N, K, L, P, T, V or S, with A, Q, D, E, N and S being preferred, the amino acid at position 24 of the E2 protein is substituted to R, D or I, with R being preferred, the amino acid at position 25 of the E2 protein is substituted to D, K, L, N, R, T, V, E or P, with D, E, K, L, N, R, T and V being preferred, the amino acid at position 41 of the E2 protein is substituted to A, N or E, and/or the amino acid at position 64 of the E2 protein is substituted to A, S, E, D, G, H, T, L, P, K or W, with A, K, and W being preferred.

In one aspect of the invention it is understood that the amino acid substitutions as described in tables 1 to 7 are the specific substitutions as described in the paragraph above. For instance, the amino acid at position 10 of the E2 protein as described in tables 1 to 7 can be substituted to A or P, the amino acid at position 14 of the E2 protein as described in tables 1 to 7 can be substituted to K, Q or R, the amino acid at position 22 of the E2 protein as described in tables 1 to 7 can be substituted to A, R, Q, E, D, N, K, L, P, T, V or S, with A, Q, D, E, N and S being preferred, the amino acid at position 24 of the E2 protein as described in tables 1 to 7 can be substituted to R, D or I, with R being preferred, the amino acid at position 25 of the E2 protein as described in tables 1 to 7 can be substituted to D, K, L, N, R, T, V, E or P, with D, K, L, N, R, T and V being preferred, the amino acid at position 41 of the E2 protein as described in tables 1 to 7 can be substituted to A, N or E, and/or the amino acid at position 64 of the E2 protein as described in tables 1 to 7 can be substituted to A, S, E, D, G, H, T, L, P, K or W, with A, K, and W being preferred. It is within the knowledge of a skilled person in the art to perform all possible permutation within the tables 1 to 7 and to combine, for instance the mutation 10A with the mutation 14K, 14Q or 14R, or to combine the mutation 10P with the mutation 14K, 14Q or 14R, etc.

In one aspect of the invention, the amino acid substitution within the 6B8 epitope of the E2 protein according to the invention results in a mutated 6B8 epitope comprising amino acid A or P at position 10 of the E2 protein, amino acid K, Q or R at position 14 of the E2 protein, amino acid A, R, Q, E, D, N, K, L, P, T, V or S at position 22 of the E2 protein, amino acid R, D or I at position 24 of the E2 protein, amino acid D, K, L, N, R, T, V, E or P at position 25 of the E2 protein, amino acid A, N or E at position 41 of the E2 protein, and/or amino acid A, S, E, D, G, H, T, L, P, K or W at position 64 of the E2 protein, with A, Q, D, E, N and S being preferred at amino acid position 22, R being preferred at amino acid position 24, D, K, L, N, R, T and V being preferred at amino acid position 25, A, K, and W being preferred at amino acid position 64 of the E2 protein.

A person skilled in the art would acknowledge that the recombinant CSFV E2 protein of the invention can be derived from various CSFV isolates, as the 6B8 epitope is evolutionarily conserved among different CSFV strains.

In one aspect of the invention, the recombinant CSFV E2 protein of the invention is derived from an isolate of genogroup 2.1. In one aspect of the invention, the recombinant CSFV E2 protein is derived for example from the field strain GD18 or QZ07. The field strain QZ07 has a full length nucleotide sequence as shown in SEQ ID NO: 13, or comprises or expresses a polyprotein with the amino acid sequence set forth in SEQ ID NO:11. The field strain GD18 has a full length nucleotide sequence as shown in SEQ ID NO: 14, or comprises or expresses a polyprotein with the amino acid sequence set forth in SEQ ID NO:12.

In one aspect of the invention, the recombinant CSFV E2 protein of the invention is derived from an isolate of genogroup 1. In one aspect of the invention, the recombinant CSFV E2 protein is derived from the C-strain well known in the art.

In one aspect of the invention, the recombinant CSFV E2 protein is derived, for example from a field strain QZ07, and comprises a substitution of Y to A or P at amino acid position 10 of the E2 protein, a substitution of S to K, Q or R at amino acid position 14 of the E2 protein, a substitution of G to A, R, Q, E, D, N, K, L, P, T, V or S, with A, Q, D, E, N and S being preferred, at amino acid position 22 of the E2 protein, a substitution of E to R, D or I, with R being preferred, at amino acid position 24 of the E2 protein, substitution of G to D, K, L, N, R, T, V, E or P, with D, K, L, N, R, T and V being preferred, at amino acid position 25 of the E2 protein, a substitution of D to A, N or E at amino acid position 41 of the E2 protein, and/or a substitution of R to A, S, E, D, G, H, T, L, P, K or W, with A, K, and W being preferred, at amino acid position 64 of the E2 protein.

In one aspect of the invention, the recombinant CSFV E2 protein is derived, for example from a field strain QZ07, and comprises a substitution of E to R, D or I, with R being preferred, at amino acid position 24 and a substitution of G to D, K, L, N, R, T, V, E or P, with D, K, L, N, R, T and V being preferred, at amino acid position 25 of the E2 protein, optionally further comprises a substitution of S to K, Q or R at amino acid position 14 of the E2 protein, or a substitution of G to A, R, Q, E, D, N, K, L, P, T, V or S, with A, Q, D, E, N and S being preferred, at amino acid position 22 of the E2 protein, and at least one further substitution selected from the group consisting of the substitution of Y to A or P at amino acid position 10 of the E2 protein, the substitution of D to A, N or E at amino acid position 41 of the E2 protein, and/or the substitution of R to A, S, E, D, G, H, T, L, P, K or W, with A, K, and W being preferred, at amino acid position 64 of the E2 protein.

In one aspect of the invention, the recombinant CSFV E2 protein is derived, for example from a field strain QZ07, and comprises a substitution of E to R, D or I, with R being preferred, at amino acid position 24, a substitution of G to D, K, L, N, R, T, V, E or P, with D, K, L, N, R, T and V being preferred, at amino acid position 25 of the E2 protein, a substitution of S to K, Q or R at amino acid position 14 of the E2 protein, a substitution of G to A, R, Q or E, with A and R being preferred, at amino acid position 22 of the E2 protein, and a substitution of R to A, S, E, D, G, H, T, L, P, K or W, with A, K, and W being preferred, at amino acid position 64 of the E2 protein.

In one aspect of the invention, the recombinant CSFV E2 protein is derived, for example from a field strain GD18, and comprises a substitution of E to R, D or I, with R being preferred, at amino acid position 24 of the E2 protein, and optionally comprises a substitution of S to K, Q or R at amino acid position 14 of the E2 protein, a substitution of G to A, R, Q, E, D, N, K, L, P, T, V or S, with A, Q, D, E, N and S being preferred, at amino acid position 22 of the E2 protein, a substitution of Y to A or P at amino acid position 10 of the E2 protein, a substitution of D to A, N or E at amino acid position 41 of the E2 protein, and/or a substitution of R to A, S, E, D, G, H, T, L, P, K or W, with A, K, and W being preferred, at amino acid position 64 of the E2 protein.

In one aspect of the invention, the recombinant CSFV E2 protein is derived, for example from a field strain GD18, and comprises a substitution of E to R, D or I, with R being preferred, at amino acid position 24 and a substitution of G to D, K, L, N, R, T, V, E or P, with D, K, L, N, R, T and V being preferred, at amino acid position 25 of the E2 protein, optionally comprises a substitution of S to K, Q or R at amino acid position 14 of the E2 protein or a substitution of G to A, R, Q, E, D, N, K, L, P, T, V or S, with A, Q, D, E, N and S being preferred, at amino acid position 22 of the E2 protein, and at least one further substitution selected from the group consisting of the substitution of Y to A or P at amino acid position 10 of the E2 protein, the substitution of D to A, N or E at amino acid position 41 of the E2 protein, and/or the substitution of R to A, S, E, D, G, H, T, L, P, K or W, with A, K, and W being preferred, at amino acid position 64 of the E2 protein.

In one aspect of the invention, the recombinant CSFV E2 protein is derived, for example from a field strain GD18, and comprises a substitution of E to R, D or I, with R being preferred, at amino acid position 24, a substitution of G to D, K, L, N, R, T, V, E or P, with D, K, L, N, R, T and V being preferred, at amino acid position 25 of the E2 protein, a substitution of S to K, Q or R at amino acid position 14 of the E2 protein, a substitution of G to A, R, Q, E, D, N, K, L, P, T, V or S, with A, Q, D, E, N and S being preferred, at amino acid position 22 of the E2 protein, and a substitution of R to A, S, E, D, G, H, T, L, P, K or W, with A, K, and W being preferred, at amino acid position 64 of the E2 protein.

In one aspect of the invention, the recombinant CSFV E2 protein is derived, for example from C-strain, and comprises a substitution of G to R, D or I, with R being preferred, at amino acid position 24 of the E2 protein, and optionally comprises a substitution of S to K, Q or R at amino acid position 14 of the E2 protein or a substitution of G to A, R, Q, E, D, N, K, L, P, T, V or S, with A, Q, D, E, N and S being preferred, at amino acid position 22 of the E2 protein, and at least one further substitution selected from the group consisting of the substitution of Y to A or P at amino acid position 10 of the E2 protein, the substitution of D to A, N or E at amino acid position 41 of the E2 protein, and/or the substitution of R to A, S, E, D, G, H, T, L, P, K or W, with A, K, and W being preferred, at amino acid position 64 of the E2 protein.

In one aspect of the invention, the recombinant CSFV E2 protein is derived, for example from C-strain, and comprises a substitution of G to R, D or I, with R being preferred, at amino acid position 24 and a substitution of G to D, K, L, N, R, T, V, E or P, with D, K, L, N, R, T and V being preferred, at amino acid position 25 of the E2 protein, and optionally r comprises a substitution of S to K, Q or R at amino acid position 14 of the E2 protein or a substitution of G to A, R, Q, E, D, N, K, L, P, T, V or S, with A, Q, D, E, N and S being preferred, at amino acid position 22 of the E2 protein, and at least one further substitution selected from the group consisting of the substitution of Y to A or P at amino acid position 10 of the E2 protein, the substitution of D to A, N or E at amino acid position 41 of the E2 protein, and/or the substitution of R to A, S, E, D, G, H, T, L, P, K or W, with A, K, and W being preferred, at amino acid position 64 of the E2 protein.

In one aspect of the invention, the recombinant CSFV E2 protein is derived, for example from C-strain, and comprises a substitution of G to R, D or I, with R being preferred, at amino acid position 24, a substitution of G to D, K, L, N, R, T, V, E or P, with D, K, L, N, R, T and V being preferred, at amino acid position 25 of the E2 protein, a substitution of S to K, Q or R at amino acid position 14 of the E2 protein, a substitution of G to A, R, Q, E, D, N, K, L, P, T, V or S, with A, Q, D, E, N and S being preferred being preferred, at amino acid position 22 of the E2 protein, and a substitution of R to A, S, E, D, G, H, T, L, P, K or W, with A, K, and W being preferred, at amino acid position 64 of the E2 protein. In one aspect of the invention, in order to obtain a soluble E2 protein, the recombinant E2 protein according to the invention may be truncated to remove the transmembrane domain. For example, the last about 40 amino acids (e.g., 42 or 43 amino acids) of the C-terminus of the intact E2 protein according to the invention may be deleted.

In one aspect of the invention, in order to obtain a secreted format of the recombinant E2 protein according to the invention, a signal peptide can be added to the N-terminal of the E2 protein. For example, the last about 20 amino acids, in particular the last 16 amino acids (e.g., for C-strain) or 21-23 amino acids (e.g., for GD18 or QZ07), from E1 protein can be added to the N-terminal of the recombinant E2 protein according to the invention. In one aspect, the signal peptide may comprise an amino acid sequence selected from SEQ ID NOs: 41-43. A person skilled in the art would acknowledge that other signal peptide allowing secret expression can also be applied in the present invention.

In one aspect of the invention, the E2 protein may be truncated to remove the transmembrane domain and a signal peptide can be added to the N-terminal of the E2 protein, so as to obtain a soluble and secreted E2 protein, for example, the last 43 amino acids of the intact E2 protein may be deleted and the last 16 amino acids or 21-23 amino acids from E1 protein can be added to the N-terminal of the E2 protein.

In one aspect of the invention, the recombinant E2 protein may also comprise a fusion tag for identification and/or purification. Such tags are well known in the art, such as a His-tag or a FLAG-tag.

In one aspect of the invention, the immunoglobulin Fc fragment can be linked to the E2 protein. The term “immunoglobulin Fc fragment”, as used herein, refers to a protein that contains the heavy-chain constant region 2 (CH2) and the heavy-chain constant region 3 (CH3) of an immunoglobulin and, more particular, that does not contain the variable regions of the heavy and light chains, and the light-chain constant region 1 (CL1) of the immunoglobulin. It may further include the hinge region, or a portion of the hinge region, of the immunoglobulin (i.e., the hinge region at the heavy-chain constant region). Also, the immunoglobulin Fc fragment may contain a part of the heavy-chain constant region 1 (CH1). It is understood that the term “immunoglobulin Fc fragment”, as used herein, is equivalent to “Fc fragment”.

The herein used term “linked to” in particular refers to any means for connecting, within a polypeptide, an immunoglobulin Fc fragment to the C-terminus or N-terminus of the E2 protein. Example of linking means may include direct linkage of the immunoglobulin Fc fragment to the E2 protein by covalent bonding. In one aspect, the Fc fragment can be linked to the C terminus of the E2 protein.

In one aspect, the immunoglobulin Fc fragment can be linked to the E2 protein via a peptide linker. The term “peptide linker” as used herein refers to a peptide comprising one or more amino acid residues. More particular, the term “peptide linker” as used herein refers to a peptide capable of connecting two variable proteins and/or domains, e.g. the E2 protein and an immunoglobulin Fc fragment. The peptide linker mentioned herein is preferably an amino acid sequence being 3 to 20 amino acid residues in length. For example, the linker moiety may be a peptide linker being 1, 2, 3, 4, 5, 6, 7, 8, 9, or 10 amino acid residues in length. The exemplary sequences of the peptide linkers can be GGS, GSGGSG, GGGGS, GGGGSGGGGS. In one aspect, the amino acid sequence of the peptide linker is GGS (SEQ ID NO: 91).

In one aspect, the immunoglobulin Fc fragment is a swine IgG Fc fragment. In one aspect, the Fc fragment can be linked to the C terminus of the E2 protein. In one aspect, the Fc fragment can be linked to the C terminus of the E2 protein via a peptide linker. In one aspect, the amino acid sequence of the swine Fc fragment is shown by SEQ ID NO: 95. The nucleotide sequence encoding the Fc fragment is shown by SEQ ID NO: 96.

In one aspect of the invention, the recombinant CSFV E2 protein comprises one of the amino acid sequences selected from the group consisting of SEQ ID NOs: 15-20, 22-33, 35-40, 49-72 and 74-81.

In one aspect of the invention, the recombinant E2 protein of the invention comprises an amino acid sequence having at least 75%, at least 80%, at least 85%, at least 90%, at least 91%, at least 92%, at least 93%, at least 94%, at least 95%, at least 96%, at least 97%, at least 98%, at least 99% sequence identity to any one of SEQ ID NOs: 15-20, 22-33, 35-40, 49-72 and 74-81 containing at least one mutation within the 6B8 epitope.

In one aspect, the present invention provides a recombinant CSFV (classical swine fever virus) comprising the recombinant E2 protein of the invention. In one aspect of the invention, the recombinant CSFV is attenuated.

A person skilled in the art would acknowledge that the recombinant CSFV of the invention can be derived from various CSFV isolates, as the 6B8 epitope is evolutionarily conserved among different CSFV strains. In one aspect of the invention, the recombinant CSFV of the invention is derived from an isolate of genogroup 2.1. In one aspect of the invention, the recombinant CSFV is derived for example from the field strain GD18 or QZ07. In one aspect of the invention, the recombinant CSFV of the invention is derived from an isolate of genogroup 1. In one aspect of the invention, the recombinant CSFV is derived from the C-strain well known in the art.

In one aspect, the present invention also provides an immunogenic composition comprising the recombinant CSFV E2 protein according to the present invention and/or the recombinant CSFV of the invention.

The term “immunogenic composition” as used herein refers to a composition that comprises at least one antigen, which elicits an immunological response in the host to which the immunogenic composition is administered. Such immunological response may be a cellular and/or antibody-mediated immune response to the immunogenic composition of the invention. The host is also described as “subject”. Preferably, any of the hosts or subjects described or mentioned herein is an animal.

Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic composition of the invention. Preferably, the host will display either a protective immunological response or a therapeutically response.

A “protective immunological response” will be demonstrated by either a reduction or lack of clinical signs normally displayed by an infected host, a quicker recovery time and/or a lowered duration of infectivity or lowered pathogen titer in the tissues or body fluids or excretions of the infected host.

An “antigen” as used herein refers to, but is not limited to, components which elicit an immunological response in a host to an immunogenic composition or vaccine of interest comprising such antigen or an immunologically active component thereof.

In case where the host displays a protective immunological response such that resistance to new infection will be enhanced and/or the clinical severity of the disease reduced, the immunogenic composition is described as a “vaccine”.

In one aspect, the immunogenic composition of the present invention is a vaccine.

The term “vaccine” as understood herein is a vaccine for veterinary use comprising antigenic substances and is administered for the purpose of inducing a specific and active immunity against a disease provoked by a CSFV infection.

Preferably, the vaccine according to the invention is a subunit CSFV vaccine, comprising a recombinant CSFV E2 protein, preferably as described herein, eliciting a protective immune response in the host animal.

A vaccine may additionally comprise further components typical to pharmaceutical compositions.

Additional components to enhance the immune response are constituents commonly referred to as “adjuvants”, or ancillary molecules added to the vaccine or generated by the body after the respective induction by such additional components, like but not restricted to interferons, interleukins or growth factors. “Adjuvants” as used herein, can include aluminum hydroxide and aluminum phosphate, saponins e.g., Quil A, QS-21 (Cambridge Biotech Inc., Cambridge MA), GPI-0100 (Galenica Pharmaceuticals, Inc., Birmingham, AL), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.), John Wiley and Sons, NY, pp51-94 (1995) and Todd et al., Vaccine 15:564-570 (1997). Exemplary adjuvants are the SPT emulsion described on page 147 of “Vaccine Design. The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8. No. 2. June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms. e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P. 934P and 971P. Most preferred is the use of Cabopol 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta GA), SAF-M (Chiron. Emeryville CA), monophosphoryl lipid A. Avridine lipid-amine adjuvant, heat-labile enterotoxin from E. coli (recombinant or otherwise), cholera toxin. IMS 1314 or muramyl dipeptide, or naturally occurring or recombinant cytokines or analogs thereof or stimulants of endogenous cytokine release, among many others.

In one aspect, the immunogenic composition is formulated into a water-in-oil emulsion with a suitable adjuvant. The adjuvant can comprise oils and surfactants. In one aspect, the adjuvant is MONTANIDE™ ISA 71R VG (Manufactured by Seppic Inc. Cat no: 365187). In one aspect, the adjuvant is Seppic ISA 206. The adjuvant can be added in an amount of about 100 μg to about 10 mg per dose. Even more preferred the adjuvant is added in an amount of about 100 μg to about 10 mg per dose. Even more preferred the adjuvant is added in an amount of about 500 μg to about 5 mg per dose. Even more preferred the adjuvant is added in an amount of about 750 μg to about 2.5 mg per dose. Most preferred the adjuvant is added in an amount of about 1 mg per dose. In one embodiment, the immunogenic composition of the invention comprises about 7 parts of oil phase containing the adjuvant and about 3 parts of aqueous phase containing the E2 protein of the invention per dose.

In one aspect of the present invention, the at least one mutation within the 6B8 epitope of the E2 protein specifically recognized by the 6B8 monoclonal antibody, as defined above, such as a substitution at amino acid position 24 of the E2 protein, a substitution at amino acid positions 24/25 of the E2 protein, a substitution at amino acid position 14 of the E2 protein, a substitution at amino acid position 22 of the E2 protein, a substitution at amino acid position 10 of the E2 protein, a substitution at amino acid position 41 of the E2 protein and/or a substitution at amino acid position 64 of the E2 protein, is used as a marker.

The term “marker” as used herein refers to the mutant 6B8 epitope according to the present invention. The mutant 6B8 epitope according to the present invention is different from the 6B8 epitope sequence of a wildtype CSFV E2 protein (6B8 epitope that has not been genetically modified). Thus, the mutant 6B8 epitope according to the present invention allows the differentiation of naturally infected animals having a non-mutated 6B8 epitope from vaccinated animals having a mutant 6B8 epitope according to the present invention by exemplary immuno tests and/or genomic analytical tests.

In one aspect of the invention, the immunogenic composition of the present invention is a marker vaccine or a DIVA (differentiation between infected and vaccinated animals) vaccine.

The term “marker vaccine” or “DIVA (differentiation between infected and vaccinated animals)” refers to a vaccine having a marker as set forth above. Thus, a marker vaccine can be used for differentiating a vaccinated animal from a naturally infected animal. The immunogenic composition of the present invention acts as a marker vaccine because, in contrast to infection with wild-type CSFV, in animals vaccinated with the vaccine of the present invention the substituted 6B8 epitope according to the present invention can be detected. By exemplary immuno tests and/or genomic analytical tests the substituted 6B8 epitope according to the present invention can be differentiated from the 6B8 epitope sequence of a wildtype CSFV (a 6B8 epitope that has not been genetically modified). Finally, the marker epitope should be specific for the pathogen in order to avoid false-positive serological results which are induced by other organisms that may appear in livestock. However, as the 6B8 epitope is evolutionarily conserved (by sequence alignment) and specific for CSFV (6B8 mAb does not bind to BVDV). Thus, the substituted 6B8 epitope according to the present invention is highly suitable to be used in a marker vaccine.

A major advantage of an efficacious marker vaccine is that it allows the detection of pigs acutely infected or infected some time (for example at least ca. 3 weeks) before taking samples in a vaccinated pig population, and thus offers the possibility to monitor the spread or re-introduction of CSFV in a pig population. Thus, it makes it possible to declare, with a certain level of confidence, that a vaccinated pig population is free of CSFV on the basis of laboratory test results.

The marker vaccine of the present invention is ideally suited for an emergency vaccination in the case of swine fever detection or outbreak. The marker vaccine facilitates fast and effective administration and allows discrimination between animals infected with the field virus (disease-associated) and vaccinated animals.

In one aspect of the present invention, the animals treated with the immunogenic composition of the present invention can be differentiated from animals infected with naturally occurring swine fever virus via analysis of samples obtained from said animals using immuno tests and/or genomic analytical tests.

The term “sample” refers to a sample of a body fluid, to a sample of separated cells or to a sample from a tissue or an organ. Samples of body fluids can be obtained by well-known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting.

The term “obtained” may comprise an isolation and/or purification step known to the person skilled in the art, preferably using precipitation, columns etc.

The term “immuno tests” and “genomic analytical tests” are specified below. However, the analysis of said “immuno tests” and “genomic analytical tests”, respectively, is the basis for differentiating animals vaccinated with the immunogenic composition according to the present invention and animals infected with the naturally occurring (disease-associated) swine fever virus.

In one aspect of the present invention said immunogenic composition is formulated for a single-dose administration.

Advantageously, the experimental data provided by the present invention disclose that a single dose administration of the immunogenic composition of the present invention reliably and effectively stimulated a protective immune response. Thus, in one aspect of the invention said immunogenic composition is formulated for and effective by a single-dose administration.

Also, the invention provides the use of the immunogenic composition of the present invention for use as a medicament.

In one aspect, the invention provides a method of preventing and/or treating diseases associated with CSFV in an animal, the method comprising the step of administering the immunogenic composition according to the invention to an animal in need thereof. In one aspect, the disease associated with CSFV is CSF.

The present invention also relates to a method for immunizing an animal, comprising administering to such animal any of the immunogenic compositions according to the present invention. The present invention also relates to a method for immunizing an animal, comprising a single administering to such animal any of the immunogenic compositions according to the present invention. Preferably, the method for immunizing an animal is effective by the single administration of the immunogenic compositions according to the present invention to such animal

The term “immunizing” relates to an active immunization by the administration of an immunogenic composition to an animal to be immunized, thereby causing an immunological response against the antigen included in such immunogenic composition.

The immunization results in lessening of the incidence of the particular CSFV infection in a herd or in the reduction in the severity of clinical signs caused by or associated with the particular CSFV infection.

Preferably, the immunization results in lessening of the incidence of the particular CSFV infection in a herd or in the reduction in the severity of clinical signs caused by or associated with the particular CSFV infection by a single administration of the immunogenic composition according to the present invention.

According to one aspect of the invention, the immunization of an animal in need with the immunogenic compositions as provided herewith, results in preventing infection of a subject by CSFV infection, preferably by a single administration of the immunogenic composition according to the present invention. Even more preferably, immunization results in an effective, long-lasting, immunological-response against CSFV infection. It will be understood that the said period of time will last more than 2 months, preferably more than 3 months, more preferably more than 4 months, more preferably more than 5 months, more preferably more than 6 months. It is to be understood that immunization may not be effective in all animals immunized. However, the term requires that a significant portion of animals of a herd are effectively immunized.

Preferably, a herd of animals is envisaged in this context which normally, i.e. without immunization, would develop clinical signs normally caused by or associated with a CSFV infection. Whether the animals of a herd are effectively immunized can be determined without further ado by the person skilled in the art. Preferably, the immunization shall be effective if clinical signs in at least 33%, at least 50%, at least 60%, at least 70%, at least 80%, or at least 90% of the animals of a given herd are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, and most preferably by at least 95% in comparison to animals that are either not immunized or immunized with an immunogenic composition that was available prior to the present invention but subsequently infected by CSFV.

In one aspect of the present invention, the animal is swine. In one aspect the animal is a piglet. Piglets are normally younger than 3 to 4 weeks of age. In one aspect the piglets are vaccinated between 1 to 4 weeks of age. In one aspect the animal is a sow. In one aspect the animal is a pregnant sow.

In one aspect of the present invention, the immunogenic composition is administered intradermal, intratracheal, intravaginal, intramuscular, intranasal, intravenous, intraarterial, intraperitoneal, oral, intrathecal, subcutaneous, intracutaneous, intracardial, intralobal, intramedullar, intrapulmonary, and combinations thereof. However, depending on the nature and mode of action of a compound, the immunogenic composition may be administered by other routes as well.

The present invention also provides a method of reducing the incidence of or severity in an animal of one or more clinical signs associated with CSF, the method comprising the step of administering the immunogenic composition according to the present invention to an animal in need thereof, wherein the reduction of the incidence of or the severity of the one or more clinical signs is relative to an animal not receiving the immunogenic composition. Preferably, the method comprises the administration of a single dose of the immunogenic composition and is effective in reduction of the incidence of or the severity of the one or more clinical signs by such single administration of the immunogenic composition.

The term “clinical signs” as used herein refers to signs of infection of an animal from CSFV. The clinical signs are defined further below. However, the clinical signs also include but are not limited to clinical signs that are directly observable from a live animal. Examples for clinical signs that are directly observable from a live animal include nasal and ocular discharge, lethargy, coughing, wheezing, thumping, elevated fever, weight gain or loss, dehydration, diarrhea, joint swelling, lameness, wasting, paleness of the skin, unthriftiness, and the like. Mittelholzer et al. (Vet.Microbiol., 2000, 74 (4): p. 293-308) developed a checklist for the determination of the clinical scores in CSF animal experiments. This checklist contains the parameters liveliness, body tension, body shape, breathing, walking, skin, eyes/conjunctiva, appetite, defecation and leftovers in feeding through.

Preferably, clinical signs are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, and most preferably by at least 95% in comparison to subjects that are either not treated or treated with an immunogenic composition that was available prior to the present invention but subsequently infected by CSFV.

In one aspect of the invention the immunogenic composition is administered once and is efficacious by such single administration.

However, while the single dose administration is preferred, the immunogenic composition can also be administered twice or several times, with a first dose being administered prior to the administration of a second (booster) dose. Preferably, the second dose is administered at least 15 days after the first dose. More preferably, the second dose is administered between 15 and 40 days after the first dose. Even more preferably, the second dose is administered at least 17 days after the first dose. Still more preferably, the second dose is administered between 17 and 30 days after the first dose. Even more preferably, the second dose is administered at least 19 days after the first dose. Still more preferably, the second dose is administered between 19 and 25 days after the first dose. Most preferably the second dose is administered at least 21 days after the first dose. In a preferred aspect of the two-time administration regimen, both the first and second doses of the immunogenic composition are administered in the same amount. In addition to the first and second dose regimen, an alternate embodiment comprises further subsequent doses. For example, a third, fourth, or fifth dose could be administered in these aspects. Preferably, subsequent third, fourth, and fifth dose regimens are administered in the same amount as the first dose, with the time frame between the doses being consistent with the timing between the first and second doses mentioned above.

In one aspect of the invention the one or more clinical signs are selected from the group consisting of: respiratory distress, labored breathing, coughing, sneezing, rhinitis, tachypnea, dyspnea, pneumonia, red/blue discolouration of the cars and vulva, jaundice, lymphocytic infiltrates, lymphadenopathy, hepatitis, nephritis, anorexia, fever, lethargy, agalatia, diarrhea, nasal extrudate, conjunctivitis, progressive weight loss, reduced weight gain, paleness of the skin, gastric ulcers, macroscopic and microscopic lesions on organs and tissues, lymphoid lesions, mortality, virus induced abortion, stillbirth, malformation of piglets, mummification and combinations thereof.

In one aspect, the present invention also provides a method of differentiating animals infected with CSFV from animals vaccinated with the immunogenic composition according to the present invention, comprising
a) obtaining a sample, and
b) testing said sample in an immuno test.

The term “immuno test” refers to a test comprising an antibody specific for the 6B8 epitope of the E2 protein of the CSFV. The antibody may be specific for the mutant 6B8 epitope according to the present invention or for the 6B8 epitope of a wildtype CSFV E2 protein (6B8 epitope that has not been genetically modified). However, the term “immuno test” does also refer to a test comprising mutant 6B8 epitope peptides according to the present invention or 6B8 epitope peptides of a wildtype CSFV E2 protein (6B8 epitope that has not been genetically modified). Examples of immuno tests include any enzyme-immunological or immunochemical detection method such as ELISA (enzyme linked immunosorbent assay), EIA (enzyme immunoassay), RIA (radioimmunoassay), sandwich enzyme immune tests, fluorescent antibody test (FAT), electrochemiluminescence sandwich immunoassays (ECLIA), dissociation-enhanced lanthanide fluoro immuno assay (DELFIA) or solid phase immune tests, immunofluorescent test (IFT), immunohistological staining, Western blot analysis or any other suitable method available to technicians skilled in the art. Depending upon the assay used, the antigens or the antibodies can be labeled by an enzyme, a fluorophore or a radioisotope. See, e.g., Coligan et al. Current Protocols in Immunology, John Wiley & Sons Inc., New York, N. Y. (1994); and Frye et al., Oncogen 4:1153-1157, 1987.

Preferably, an antibody specific for the 6B8 epitope of a wildtype CSFV E2 protein is used to detect CSFV antigen in serum cells (such as leucocytes) or cryostat sections of isolated organs (such as tonsils, spleen, kidney, lymph nodes, distal portions of the ileum) from an animal (such as a pig) that is suspected to be infected with wildtype CSFV or that is vaccinated with a vaccine comprising a recombinant CSFV E2 protein according to the invention. In such a case, only the sample of the animal infected with wildtype CSFV will show positive results by said 6B8 epitope specific antibody. In contrast, the sample of an animal vaccinated with the vaccine comprising a recombinant CSFV E2 protein of the present invention will show no results by said 6B8 epitope specific antibody due to the mutation within the 6B8 epitope according to the present invention. In an alternative test, CSFV is isolated from, for example, organs (such as the tonsils of an animal) or serum cells (such as leukoyctes) infected, suspected to be infected with wildtype CSFV or vaccinated animals and incubated with a suitable cell line (such as SK-6 cells or PK-15 cells) for infection of the cells with the virus. The replicated virus is subsequently detected in the cells using 6B8 epitope specific antibodies that differentiate between the field (wildtype, disease associated) CSFV and the recombinant CSFV according to the invention. Further, peptides could be used to block unspecific cross-reactivity. Moreover, antibodies specific for other epitopes of the wildtype CSFV could be used as a positive control.

More preferably, an ELISA is used, wherein the antibody specific for the 6B8 epitope of a wildtype CSFV E2 protein (6B8 epitope that has not been genetically modified) is cross-linked to micro-well assay plates for differentiating between infected pigs from pigs vaccinated with the vaccine according to the present invention. Said cross-linking preferably is performed through an anchor protein such as, for example, poly-L-lysine. ELISAs employing such cross-linking are in general more sensitive when compared to ELISAs employing a passively coated technique. The wildtype (disease associated) CSFV binds to the antibody specific for the 6B8 epitope of a wildtype CSFV E2 protein (6B8 epitope that has not been genetically modified). The detection of the binding of the wildtype CSFV virus to the antibody specific for the 6B8 epitope of a wildtype CSFV can be performed by a further antibody specific for CSFV. In such a case, only the sample of the infected pig will show positive results by the 6B8 epitope specific antibody. Further, peptides could be used to block unspecific cross-reactivity. Moreover, antibodies specific for other epitopes of the wildtype CSFV could be used as a positive control.

Alternatively, the micro-well assay plates may be cross-linked with an antibody specific for CSFV other than the antibody specific for the 6B8 epitope of a wildtype CSFV E2 protein (6B8 epitope that has not been genetically modified). The wildtype (disease associated) CSFV binds to the cross linked antibody. The detection of the binding of the wildtype CSFV to the cross linked antibody can be performed by the antibody specific for the 6B8 epitope of a wildtype CSFV E2 protein (6B8 epitope that has not been genetically modified).

As already set forth above the 6B8 epitope is evolutionarily conserved and specific for wildtype CSFV.

Therefore, more preferably, an ELISA is used for detecting in the sample antibodies that are directed against the mutant 6B8 epitope according to the present invention or the 6B8 epitope of a wildtype CSFV (6B8 epitope that has not been genetically modified). Such a test comprises mutant 6B8 epitope peptides according to the present invention or the 6B8 epitope peptides of a wildtype CSFV (6B8 epitope that has not been genetically modified).

Such a test could e.g., comprise wells with a substituted 6B8 epitope according to the present invention or the 6B8 epitope of a wildtype CSFV (6B8 epitope that has not been genetically modified) cross-linked to micro-well assay plates. Said cross-linking preferably is performed through an anchor protein such as, for example, poly-L-lysine. Expression systems for obtaining a mutant or wildtype 6B8 epitope are well known to the person skilled in the art. Alternatively, said 6B8 epitopes could be chemically synthesized. It has to be understood that although the mutant or wildtype 6B8 epitope as such can be used in a test according to the invention, it can be convenient to use a protein comprising the complete E2 protein or a fragment of the E2 protein comprising the said 6B8 epitope, instead of the relatively short epitope as such. Especially when the epitope is for example used for the coating of a well in a standard ELISA test, it may be more efficient to use a larger protein comprising the epitope, for the coating step.

Animals vaccinated with the vaccine comprising a recombinant CSFV E2 protein according to the present invention have not raised antibodies against the wild-type 6B8 epitope. However, such animals have raised antibodies against the substituted 6B8 epitope according to the present invention. As a consequence, no antibodies bind to a well coated with the wildtype 6B8 epitope. In contrast, if a well has been coated with the mutant 6B8 epitope according to the present invention antibodies bind to said mutant 6B8 epitope.

Animals infected with the wild-type CSFV will however have raised antibodies against the wild-type epitope of CSFV. However, such animals have not raised antibodies against the mutant 6B8 epitope according to the present invention. As a consequence, no antibodies bind to a well coated with the mutant 6B8 epitope according to the present invention. In contrast, if a well has been coated with the wildtype 6B8 epitope antibodies bind to the wildtype 6B8 epitope.

The binding of the antibodies to the mutant 6B8 epitope according to the present invention or the 6B8 epitope of a wildtype CSFV (6B8 epitope that has not been genetically modified) can be done by methods well known to the person skilled in the art.

Preferably, the ELISA is a sandwich type ELISA. More preferably, the ELISA is a competitive ELISA. Most preferably, the ELISA is a double competitive ELISA. However, the different ELISA techniques are well known to the person skilled in the art. ELISA have been described exemplary by Wensvoort G. et al., 1988 (Vet. Microbiol. 17 (2): 129-140), by Robiolo B. et al., 2010 (J. Virol. Methods. 166 (1-2): 21-27) and by Colijn. E. O. et al., 1997 (Vet. Microbiology 59:15-25).

In one aspect of the present invention the immuno test comprises testing whether antibodies specifically recognizing the intact 6B8 epitope of the CSFV E2 protein are binding to the CSFV E2 protein in the sample. In one aspect of the present invention the immuno test comprises testing whether an antibody specifically recognizing a 6B8 epitope of the CSFV E2 protein is present in the sample, and/or testing whether an antibody specifically recognizing a mutated 6B8 epitope of the CSFV E2 protein is present in the sample. Such a mutated 6B8 epitope comprises mutation(s) in the 6B8 epitope as defined herein.

In one aspect of the present invention the immunological test is an EIA (enzyme immunoassay) or ELISA (enzyme linked immunosorbent assay). In one aspect of the present invention the ELISA is an indirect ELISA, Sandwich ELISA, a competitive ELISA or double competitive ELISA, preferably a double competitive ELISA.

In one aspect, the present invention also provides a nucleic acid coding for the recombinant CSFV E2 protein according to the present invention.

The term “nucleic acid” refers to polynucleotides including DNA molecules, RNA molecules, cDNA molecules or derivatives. The term encompasses single as well as double stranded polynucleotides.

The nucleic acid of the present invention encompasses recombinant polynucleotides (i.e. recombinant from its natural context) and genetically modified forms. Moreover, comprised are also chemically modified polynucleotides including naturally occurring modified polynucleotides such as glycosylated or methylated polynucleotides or artificial modified one such as biotinylated polynucleotides. Further, it is to be understood that the recombinant CSFV E2 protein of the present invention may be encoded by a large number of polynucleotides due to the degenerated genetic code. In one aspect of the present invention, the nucleic acid coding for the recombinant CSFV E2 protein according to the present invention is codon-optimized according to the host cell into which the nucleic acid is to be introduced.

In one aspect, the present invention also provides a vector comprising the nucleic acid coding for the recombinant CSFV E2 protein according to the present invention. In one aspect, the vector is an expression vector.

The term “vector” encompasses phage, plasmid, viral or retroviral vectors as well artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the nucleic acid of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells. More preferably, the polynucleotide is operatively linked to expression control sequences allowing expression in prokaryotic or eukaryotic cells or isolated fractions thereof. Expression of said polynucleotide comprises transcription of the polynucleotide, preferably into a translatable mRNA, Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise. e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer. SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. For example, the techniques are described in Sambrook.

Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Preferably, the vector of the invention is a baculovirus vector. More preferably, the vector of the invention is a mammalian expression vector for the expression in a mammalian cell, such as a CHO cell.

In one aspect, the invention also provides a host cell comprising the nucleic acid or vector of the invention. The host cell may be a prokaryotic cell, such as E. coli, or an eukaryotic cell, such as for example an inset cell. Preferably, the host cell is an SF9 cell. More preferably, the host cell is a mammalian cell, such as a CHO cell.

In one aspect, the invention also provides a method for producing a recombinant CSFV E2 protein, preferably the recombinant CSFV E2 protein comprising the one or more amino acid substitutions as defined in tables 1 to 7 or disclosed otherwise herein of, comprising
(i) culturing the host cell, preferably the CHO cell as defined herein under conditions suitable for the expression of the CSFV E2 protein, and
(ii) isolating and optionally purifying the CSFV E2 protein.

The purification of the CSFV E2 protein, for example can be done via a fusion tag or fusion peptide attached to CSFV E2 protein, e.g. via a His-tag, FLAG-tag or a Fc-fragment, if present.

In one aspect, the invention also provides a method of preparing an immunogenic composition, comprising: (i) culturing cells containing an expression vector capable of expressing an E2 protein; and (ii) harvesting the E2 protein or the whole cell culture comprising the E2 protein, wherein the E2 protein comprises at least one mutation within the 6B8 epitope of the E2 protein specifically recognized by the 6B8 monoclonal antibody as defined herein above.

In one aspect of the invention, the expression vector is a recombinant mammalian expression vector comprising the nucleic acid molecule of the invention. In one aspect, the recombinant mammalian expression vector is derived from a commercial product. In one aspect, the recombinant mammalian expression vector is derived from pcDNA3.4 (Invitrogen). In one aspect, the cells are mammalian cells. In one aspect, the mammalian cells are CHO cells.

In one aspect, the method is performed by using the ExpiCHO™ Expression System (Gibico, Cat #A29133) according to the user manual (Revision D.0, May 25, 2018, Thermo Fisher Scientific Inc). In one embodiment, the CHO cells are ExpiCHO-S™ cells sold under Cat #A29127 by Gibico. In one embodiment, the method comprises a step of infecting the mammalian cells with the recombinant mammalian expression vector of the invention, for example, using ExpiFectamine™ CHO Transfection Kit (Gibico, Cat #A29129).

In one aspect of the invention, the expression vector is a recombinant baculovirus comprising the nucleic acid molecule of the invention. In one aspect, the recombinant baculovirus is derived from a commercial product. In one aspect, the recombinant baculovirus is derived from a commercial product sold under the trademark Sapphire™ Baculovirus (Allele Biotechnology). In one aspect, the cells are insect cells. In one aspect, the insect cells are SF+ cells. In one embodiment, the SF+ cells are a commercial product sold by Protein Sciences Corporation (Meriden, CT).

In one aspect of the invention, the method comprises a step of preparing a recombinant baculovirus comprising the nucleic acid molecule of the invention. In one aspect, the recombinant baculovirus is derived from a commercial product. In one aspect, the recombinant baculovirus is derived from a commercial product sold under the trademark Sapphire™ Baculovirus (Allele Biotechnology).

In one aspect of the invention, the method comprises a step of infecting cells with the recombinant baculovirus of the invention. In one embodiment, the cells are insect cells. In one embodiment, the insect cells are SF+ cells. In one embodiment, the SF+ cells are a commercial product sold by Protein Sciences Corporation (Meriden, CT).

In one aspect of the invention, the method comprises preparing a recombinant baculovirus comprising the nucleic acid molecule of the invention, and infecting insect cells with the recombinant baculovirus. In one embodiment, the recombinant baculovirus is derived from a commercial product sold under the trademark Sapphire™ Baculovirus (Allele Biotechnology). In one embodiment, the insect cells are SF+ cells. In one embodiment, the SF+ cells are a commercial product sold by Protein Sciences Corporation (Meriden, CT).

In one aspect of the invention, the method comprises: (i) preparing a recombinant baculovirus comprising the nucleic acid molecule of the invention; (ii) infecting insect cells with the recombinant baculovirus; (iii) culturing the insect cells in a culture medium; and (iv) harvesting the E2 protein of the invention or the whole cell culture comprising the E2 protein of the invention. In one aspect, the recombinant baculovirus is derived from a commercial product sold under the trademark Sapphire™ Baculovirus (Allele Biotechnology). In one embodiment, the insect cells are SF+ cells. In one embodiment, the SF+ cells are a commercial product sold by Protein Sciences Corporation (Meriden, CT).

In one aspect of the invention, the culture medium for culturing the cells of the invention will be determined by those of skill in the art. In one aspect, the culture medium is a serum-free insect cell medium. In one aspect, the culture medium is Ex-CELL 420 (Ex-CELL® 420 serum-free medium for insect cells, Sigma-Aldrich, Cat. 14420C).

In one aspect of the invention, the insect cells are cultured under the condition suitable for the expression of the E2 protein. In one aspect, the insect cells are incubated over a period of up to ten days, preferably from about two days to about ten days, more preferably from about four days to about nine days, and even more preferably from about five days to about eight days. In one aspect, the condition suitable for culturing the insect cell comprises a temperature between about 22-32° C., preferably from about 24-30° C., more preferably from about 25-29° C., even more preferably from about 26-28° C., and most preferably about 27° C.

In one aspect of the invention, the method further comprises a step of inactivating the cell culture of the invention. Any conventional inactivation method can be used for purposes of the invention, including but not limited to chemical and/or physical treatments.

In one aspect, the inactivation step comprises the addition of cyclized binary ethylenimine (BEI), preferably in a concentration of about 1 to about 20 mM, preferably of about 2 to about 10 mM, more preferably of about 5 mM or 10 mM. In one embodiment, the inactivation step comprises the addition of a solution of 2-bromoethyleneamine hydrobromide which will be cyclized to form BEI in NaOH.

In one aspect, the inactivation step is performed between 25-40° C., preferably between 28-39° C., more preferably between 30-39° C., more preferably between 35-39° C. In one embodiment, inactivation step is performed for 24-72 h, preferably for 30-72 h, more preferably 48-72 h. In general, the inactivation step is performed until no replication of the viral vector is detectable.

In one aspect of the invention, the method further comprises a step of a neutralization step after the inactivation step. The neutralization step comprises adding of an equivalent amount of an agent that neutralizes the inactivation agent within the solution. In one embodiment, the inactivation agent is BEI. In one aspect, the neutralization agent is sodium thiosulfate. In one aspect, when the inactivation agent is BEI, an equivalent amount of sodium thiosulfate will be added. For example, in the event BEI is added to a final concentration of 5 mM, a 1.0M sodium thiosulfate solution is added to give a final minimum concentration of 5 mM to neutralize any residual BEI. In one aspect, the neutralization step comprises adding of a sodium thiosulfate solution to a final concentration of 1 to 20 mM, preferably of 2 to 10 mM, more preferably of 5 mM or 10 mM, when the inactivation agent is BEI. In one aspect, the neutralization agent is added after the inactivation step is completed, which means that no replication of the viral vector replication can be detected. In one aspect, the neutralization agent is added after the inactivation step is performed for 24 h. In one aspect, the neutralization agent is added after the inactivation step is performed for 30 h. In one aspect, the neutralization agent is added after the inactivation step is performed for 48 h. In one aspect, the neutralization agent is added after the inactivation step is performed for 72 h.

In one aspect of the invention, the CSFV E2 protein is further purified. For example, the CSFV E2 protein according to the invention may comprise a fusion peptide such as for example a His-tag, a FLAG-tag or a Fc-fragment, which are attached to the CSFV E2 protein. CSFV E2 proteins with a His-tag can for example be purified by Ni-NTA affinity (nickel²⁺-ion coupled to nitrilotriacetic acid). CSFV E2 proteins with a FLAG-tag (DYKDDDDK) can for example be purified by a monoclonal antibody specifically binds to the FLAG-tag (commercial kits for example are available from Sigma-Aldrich (Anti-FLAG® MI Agarsose affinity gel)). CSFV E2 proteins linked to a Fc-fragment can for example be purified either by protein A or protein G affinity.

In one aspect, the invention also provides a method for producing a recombinant CSFV E2 protein, preferably the recombinant CSFV E2 protein comprising the one or more amino acid substitutions as defined in tables 1 to 7 or disclosed otherwise herein of, in CHO cells, said method comprises
a) adapting CHO cells to high-density suspension culture in a medium;
b) transfecting the adapted CHO cells from step a) with a recombinant mammalian expression vector comprising a nucleic acid molecule encoding the recombinant CSFV E2 protein, preferably the recombinant CSFV E2 protein comprising the one or more amino acid substitutions as defined in tables 1 to 7 or disclosed otherwise herein of,
c) culturing the transfected CHO cells from step b) under conditions suitable for the expression of the recombinant CSFV E2 protein;
d) harvesting and optionally purifying the recombinant CSFV E2 protein.

In one aspect, the invention also provides a method for producing a recombinant CSFV E2 protein, preferably the recombinant CSFV E2 protein comprising the one or more amino acid substitutions as defined in tables 1 to 7 or the recombinant CSFV E2 protein according to the present invention, in CHO cells, said method comprises
a) growing CHO cells adapted to high-density suspension culture in a medium;
b) transfecting the CHO cells from step a) with a mammalian expression vector comprising the nucleic acid molecule encoding the recombinant CSFV E2 protein, preferably the recombinant CSFV E2 protein comprising the one or more amino acid substitutions as defined in tables 1 to 7 or the recombinant CSFV E2 protein according to the present invention,
c) culturing the transfected CHO cells from step b) under conditions suitable for the expression of the recombinant CSFV E2 protein;
d) harvesting and optionally purifying the recombinant CSFV E2 protein.

In one embodiment, the recombinant mammalian expression vector is derived from pcDNA3.4 (Invitrogen).

In one embodiment, the CHO cells are ExpiCHO-S™ cells sold under Cat #A29127 by Gibico.

In one embodiment, the medium as used in the method is the ExpiCHO™ Expression Medium sold under Cat #A29100 by Gibico.

In one embodiment, the adapted CHO cells are transfected by using ExpiFectamine™ CHO Transfection Kit (Gibico, Cat #A29129).

In one embodiment, in step c), the transfected CHO cells are cultured at about 32-37° C., preferably about 32° C.

In one embodiment, in step c), the transfected CHO cells are cultured with a humidified atmosphere of about 5-8% CO₂.

In one embodiment, ExpiFectamine™ CHO Enhancer and ExpiCHO™ Feed are added in step c).

In one embodiment, the recombinant CSFV E2 protein is harvested about 2-14 days post transfection, for example, about 4-12 days post transfection, or about 8-10 days post transfection.

In one aspect of the invention, the CSFV E2 protein is further purified. For example, the CSFV E2 protein according to the invention may comprise a fusion peptide such as for example a His-tag, a FLAG-tag or an Fc-fragment, which are attached to the CSFV E2 protein. CSFV E2 proteins with a His-tag can for example be purified by Ni-NTA affinity (nickel²⁺-ion coupled to nitrilotriacetic acid). CSFV E2 proteins with a FLAG-tag (DYKDDDDK) can for example be purified by a monoclonal antibody specifically binds to the FLAG-tag (commercial kits for example are available from Sigma-Aldrich (Anti-FLAG® MI Agarsose affinity gel)). CSFV E2 proteins linked to an Fc-fragment can for example be purified either by protein A or protein G affinity.

In one aspect, the present invention provides a kit for differentiating animals infected with CSFV from animals vaccinated with the immunogenic composition of the invention. In one aspect, the kit comprises the antibody as defined herein or an antigen-binding fragment thereof, the recombinant E2 protein of the invention with mutation(s) in the 6B8 epitope, and/or a wild type E2 protein of CSFV comprising the 6B8 epitope as defined herein. The kit may also contain instructions for use.

The following clauses are also described herein and part of disclosure of the invention:
1. A recombinant CSFV (classical swine fever virus) E2 protein comprising at least one mutation within the 6B8 epitope, wherein the unmodified 6B8 epitope is specifically recognized by the 6B8 monoclonal antibody.
2. The recombinant CSFV E2 protein according to clause 1, wherein the at least one mutation within the 6B8 epitope of the E2 protein leads to a specific inhibition of the binding of a 6B8 monoclonal antibody to such mutated 6B8 epitope.
3. The recombinant CSFV E2 protein according to clause 1 or 2, wherein the 6B8 monoclonal antibody
(i) is produced by a hybridoma deposited at CCTCC under the accession number CCTCC C2018120, or
(ii) comprises a heavy chain variable region (V_{H}) having an amino acid sequence as set forth in SEQ ID NO: 7 and a light chain variable region (V_{L}) having an amino acid sequence as set forth in SEQ ID NO: 8, or
(iii) comprises the CDRs of the monoclonal antibody produced by a hybridoma deposited at CCTCC under the accession number CCTCC C2018120, or
(iv) comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO: 4, a VL CDR2 comprising the amino acid sequence set forth in SEQ ID NO:5, and a VL CDR3 comprising the amino acid sequence set forth in SEQ ID NO:6.
4. The recombinant CSFV E2 protein according to any one of clauses 1 to 3, wherein the 6B8 epitope of the E2 protein specifically recognized by the 6B8 monoclonal antibody is defined at least by the amino acid residue at position 10, position 14, position 22, position 24, positions 24/25, position 41 and/or position 64 of the E2 protein.
5. The recombinant CSFV E2 protein according to any one of clauses 1 to 3, wherein the 6B8 epitope of the E2 protein specifically recognized by the 6B8 monoclonal antibody is defined at least by the amino acid residue S14, G22, E24, E24/G25, Y10, D41 and/or R64 of the E2 protein, or is defined at least by the amino acid residue S14, G22, G24, G24/G25, Y10, D41 and/or R64 of the E2 protein.
6. The recombinant CSFV E2 protein according to any one of clauses 1 to 5, which comprises a substitution at amino acid position 24 of the E2 protein, a substitution at amino acid positions 24/25 of the E2 protein, a substitution at amino acid position 14 of the E2 protein, a substitution at amino acid position 22 of the E2 protein, a substitution at amino acid position 10 of the E2 protein, a substitution at amino acid position 41 of the E2 protein, and/or a substitution at amino acid position 64 of the E2 protein.
7. The recombinant CSFV E2 protein according to any one of clauses 1 to 6, in which the amino acid at position 24 of the E2 protein is substituted to R, the amino acid at positions 24 and 25 of the E2 protein is substituted to R and D respectively, the amino acid at position 14 of the E2 protein is substituted to K, Q or R, the amino acid at position 22 of the E2 protein is substituted to A, R, Q or E, with A and R being preferred, the amino acid at position 10 of the E2 protein is substituted to A or P, the amino acid at position 41 of the E2 protein is substituted to A, N or E, and/or the amino acid at position 64 of the E2 protein is substituted to A, S or E, with A and S being preferred.
8. The recombinant CSFV E2 protein according to any one of clauses 1 to 7, which comprises a substitution of E or G to R, D or I, with R being preferred, at amino acid position 24 of the E2 protein, a substitution of E or G to R, D or I, with R being preferred, at amino acid position 24 and G to D, K, L, N, R, T, V, E or P, with D, K, L, N, R, T and V being preferred, at amino acid position 25 of the E2 protein, a substitution of S to K, Q or R at amino acid position 14 of the E2 protein, a substitution of G to A, R, Q, E, D, N, K, L, P, T, V or S, with A, Q, D, E, N and S being preferred, at amino acid position 22 of the E2 protein, a substitution of Y to A or P at amino acid position 10 of the E2 protein, a substitution of D to A, N or E at amino acid position 41 of the E2 protein, and/or a substitution of R to A, S, E, D, G, H, T, L, P, K or W, with A, K, and W being preferred, at amino acid position 64 of the E2 protein.
9. The recombinant CSFV E2 protein according to any one of clauses 1 to 8, wherein the amino acid substitution within the 6B8 epitope results in a mutated 6B8 epitope comprising amino acid R, D or I at position 24 of the E2 protein, amino acid D, K, L, N, R, T, V, E or P at position 25 of the E2 protein, amino acid K, Q or R at position 14 of the E2 protein, amino acid A, R, Q, E, D, N, K, L, P, T, V or S at position 22 of the E2 protein, amino acid A or P at position 10 of the E2 protein, amino acid A, N or E at position 41 of the E2 protein, and/or amino acid A, S, E, D, G, H, T, L, P, K or W at position 64 of the E2 protein.
10. The recombinant CSFV E2 protein according to any one of clauses 1 to 9, wherein the recombinant CSFV E2 protein is derived from C-strain or a field strain QZ07 or GD18.
11. The recombinant CSFV E2 protein according to any one of clauses 1 to 10, wherein the recombinant CSFV E2 protein is derived from a field strain QZ07, and comprises a substitution of E to R, D or I, with R being preferred, at amino acid position 24 of the E2 protein, or a substitution of E to R at amino acid position 24 and G to D, K, L, N, R, T, V, E or P, with D, K, L, N, R, T and V being preferred, at amino acid position 25 of the E2 protein, optionally further comprises a substitution of S to K, Q or R at amino acid position 14 of the E2 protein or a substitution of G to A, R, Q, E, D, N, K, L, P, T, V or S, with A, Q, D, E, N and S being preferred, at amino acid position 22 of the E2 protein, and at least one further substitution selected from the group of consisting of the substitution of Y to A or P at amino acid position 10 of the E2 protein, the substitution of D to A, N or E at amino acid position 41 of the E2 protein, and/or the substitution of R to A, S, E, D, G, H, T, L, P, K or W, with A, K, and W being preferred, at amino acid position 64 of the E2 protein.
12. The recombinant CSFV E2 protein according to any one of clauses 1 to 10, wherein the recombinant CSFV E2 protein is derived from a field strain GD18, and comprises a substitution of E to R, D or I, with R being preferred, at amino acid position 24 of the E2 protein, or a substitution of E to R, D or I, with R being preferred, at amino acid position 24 and G to D, K, L, N, R, T, V, E or P, with D, K, L, N, R, T and V being preferred, at amino acid position 25 of the E2 protein, optionally further comprises a substitution of S to K, Q or R at amino acid position 14 of the E2 protein or a substitution of G to A, R, Q, E, D, N, K, L, P, T, V or S, with A, Q, D, E, N and S being preferred, at amino acid position 22 of the E2 protein, and at least one further substitution selected from the group of consisting of the substitution of Y to A or P at amino acid position 10 of the E2 protein, the substitution of D to A, N or E at amino acid position 41 of the E2 protein, and/or a substitution of R to A, S, E, D, G, H, T, L, P, K or W, with A, K, and W being preferred, at amino acid position 64 of the E2 protein.
13. The recombinant CSFV E2 protein according to any one of clauses 1 to 10, wherein the recombinant CSFV E2 protein is derived from C-strain, and comprises a substitution of G to R, D or I, with R being preferred, at amino acid position 24 of the E2 protein, and a substitution of G to D, K, L, N, R, T, V, E or P, with D, K, L, N, R, T and V being preferred, at amino acid position 25 of the E2 protein, and optionally further comprises a substitution of S to K at amino acid position 14 of the E2 protein or a substitution of G to A, R, Q, E, D, N, K, L, P, T, V or S, with A, Q, D, E, N and S being preferred, at amino acid position 22 of the E2 protein, and at least one further substitution selected from the group of consisting of the substitution of Y to A or P at amino acid position 10 of the E2 protein, the substitution of D to A, N or E at amino acid position 41 of the E2 protein, and/or the substitution of R to A, S, E, D, G, H, T, L, P, K or W, with A, K, and W being preferred, at amino acid position 64 of the E2 protein.
14. The recombinant CSFV E2 protein according to any one of clauses 1 to 10 wherein the recombinant CSFV E2 protein comprises one or more amino acid substitutions at the amino acid positions of the E2 protein as defined in the tables 1 to 7.
15. The recombinant CSFV E2 protein according to any one of clauses 1 to 10, wherein the recombinant CSFV E2 protein comprises one of the amino acid sequences selected from the group consisting of SEQ ID NOs: 15-20, 22-33, 35-40, 49-72 and 74-81.
16. The recombinant CSFV E2 protein according to any one of clauses 1 to 15, wherein an Fc fragment, such as a swine Fc fragment is linked to the recombinant CSFV E2 protein; preferably the Fc fragment, such as a swine Fc fragment is linked to the C terminus of the E2 protein, preferably via a peptide linker.
17. A recombinant nucleic acid coding for the recombinant CSFV E2 protein according to any one of clauses 1 to 16.
18. A vector comprising the nucleic acid of clause 17, preferably, the vector is a mammalian expression vector.
19. A host cell comprising the nucleic acid of clause 17 or the vector of clause 18, preferably, the host cell is a CHO cell.
20. A recombinant CSFV comprising the recombinant CSFV E2 protein according to any one of clauses 1 to 16.
21. The recombinant CSFV of claim 20, wherein the recombinant CSFV is attenuated.
22. An immunogenic composition comprising the recombinant CSFV E2 protein according to any one of clauses 1 to 16, the recombinant nucleic acid according to clause 17, the vector according to clause 18, the host cell of clause 19, and/or the recombinant CSFV of clause 20 or 21.
23. The immunogenic composition according to clause 22, wherein said immunogenic composition is a vaccine, preferably a marker vaccine or a DIVA (differentiation between infected and vaccinated animals) vaccine.
24. An immunogenic composition according to clause 22 or 23 for use in a method of preventing and/or treating diseases associated with CSFV in an animal, the method comprising the step of administering the immunogenic composition according to clause 22 or 23 to an animal.
25. The immunogenic composition according to clause 22 or 23 for use in a method of preventing and/or treating diseases associated with CSFV in an animal according to clause 22 or 23, wherein said animal is swine.
26. The immunogenic composition according to clause 22 or 23 for use in a method of preventing and/or treating diseases associated with CSFV in an animal according to clause 22 or 23, wherein said animal is a piglet.
27. The immunogenic composition according to clause 22 or 23 for use in a method of preventing and/or treating diseases associated with CSFV in an animal according to clause 22 or 23, wherein said animal is a piglet of 1 to 4 weeks of age.
28. The immunogenic composition according to clause 22 or 23 for use in a method of preventing and/or treating diseases associated with CSFV in an animal according to clause 22 or 23, wherein said animal is a sow.
29. The immunogenic composition according to clause 22 or 23 for use in a method of preventing and/or treating diseases associated with CSFV in an animal according to clause 22 or 23, wherein said animal is a pregnant sow.
30. The immunogenic composition according to clause 22 or 23 for use in a method of preventing and/or treating diseases associated with CSFV in an animal according to clause 22 or 23, wherein said immunogenic composition is administered only once.
31. The immunogenic composition according to clause 22 or 23 for use in a method of preventing and/or treating diseases associated with CSFV in an animal according to clause 22 or 23, wherein said immunogenic composition is administered only once to the animal and effective in preventing and/or treating diseases associated with CSFV after said single administration of the immunogenic composition.
32. The immunogenic composition according to clause 22 or 23 for use in a method of preventing and/or treating diseases associated with CSFV in an animal according to clause 22 or 23, wherein said immunogenic composition is administered one or several times.
33. The immunogenic composition according to clause 22 or 23 for use in a method of preventing and/or treating diseases associated with CSFV in an animal according to clause 22 or 23, wherein said immunogenic composition is administered one or several times to the animal and effective in preventing and/or treating diseases associated with CSFV after said single or multiple administration of the immunogenic composition.
34. A method of preventing and/or treating diseases associated with CSFV in an animal, the method comprising the step of administering the immunogenic composition according to clause 22 or 23 to an animal in need thereof.
35. A method of differentiating animals infected with CSFV from animals vaccinated with the immunogenic composition of any one of clause 22 or 23, comprising
a) obtaining a sample, and
b) testing said sample in an immuno test.
36. The method according to clause 35, wherein the immuno test comprises testing whether an antibody specifically recognizing the 6B8 epitope of the CSFV E2 protein or an antigen-binding fragment thereof can bind to the CSFV E2 protein in the sample.
37. The method according to clause 35 or 36, wherein the immuno test comprises testing whether an antibody specifically recognizing a 6B8 epitope of the CSFV E2 protein is present in the sample, and/or testing whether an antibody specifically recognizing a mutated 6B8 epitope of the recombinant CSFV E2 protein is present in the sample.
38. The method according to any one of clauses 35 to 37, wherein the immuno test is an EIA (enzyme immunoassay) or ELISA (enzyme linked immunosorbent assay), preferably a double competitive ELISA.
39. The method according to any one of clauses 36 to 38, wherein the antibody specifically recognizing the 6B8 epitope
(i) is produced by a hybridoma deposited at CCTCC under the accession number CCTCC C2018120, or
(ii) comprises a heavy chain variable region (V_{H}) having an amino acid sequence as set forth in SEQ ID NO: 7 and a light chain variable region (V_{L}) having an amino acid sequence as set forth in SEQ ID NO: 8, or
(iii) comprises the CDRs of the monoclonal antibody produced by a hybridoma deposited at CCTCC under the accession number CCTCC C2018120, or
(iv) comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO: 4, a VL CDR2 comprising the amino acid sequence set forth in SEQ ID NO:5, and a VL CDR3 comprising the amino acid sequence set forth in SEQ ID NO:6.
40. A kit for differentiating animals infected with CSFV from animals vaccinated with the immunogenic composition of any one of clause 22 or 23, which comprises an antibody specifically recognizing the 6B8 epitope of the CSFV E2 protein or an antigen-binding fragment thereof.
41. A method for producing the recombinant CSFV E2 protein according to any one of clauses 1 to 16, comprising
(i) culturing the host cell of clause 19 under conditions suitable for the expression of the CSFV E2 protein, and
(ii) isolating and optionally purifying the CSFV E2 protein.
42. A method for producing a recombinant CSFV E2 protein, preferably the recombinant CSFV E2 protein comprising the one or more amino acid substitutions as defined in tables 1 to 7 or the recombinant CSFV E2 protein according to any one of clauses 1 to 16, in CHO cells, said method comprises
a) adapting CHO cells to high-density suspension culture in a medium;
b) transfecting the adapted CHO cells from step a) with a mammalian expression vector comprising the nucleic acid molecule encoding the recombinant CSFV E2 protein, preferably the recombinant CSFV E2 protein comprising the one or more amino acid substitutions as defined in tables 1 to 7 or the recombinant CSFV E2 protein according to any one of clauses 1 to 16,
c) culturing the transfected CHO cells from step b) under conditions suitable for the expression of the recombinant CSFV E2 protein;
d) harvesting and optionally purifying the recombinant CSFV E2 protein.
43. A method for producing a recombinant CSFV E2 protein, preferably the recombinant CSFV E2 protein comprising the one or more amino acid substitutions as defined in tables 1 to 7 or the recombinant CSFV E2 protein according to any one of clauses 1 to 16, in CHO cells, said method comprises
a) growing CHO cells adapted to high-density suspension culture in a medium;
b) transfecting the CHO cells from step a) with a mammalian expression vector comprising the nucleic acid molecule encoding the recombinant CSFV E2 protein, preferably the recombinant CSFV E2 protein comprising the one or more amino acid substitutions as defined in tables 1 to 7 or the recombinant CSFV E2 protein according to any one of clauses 1 to 16,
c) culturing the transfected CHO cells from step b) under conditions suitable for the expression of the recombinant CSFV E2 protein;
d) harvesting and optionally purifying the recombinant CSFV E2 protein.
44. The method of clauses 42 or 43, wherein in step c), the transfected CHO cells are cultured at about 32-37° C., preferably about 32° C.
45. The method of any one of clauses 42-44, wherein in step c), the transfected CHO cells are cultured with a humidified atmosphere of about 5-8% CO₂.
46. The method of any one of clauses 42-45, wherein the recombinant CSFV E2 protein is harvested about 2-14 days post transfection, for example, about 4-12 days post transfection, or about 8-10 days post transfection.
47. The method of any one of clauses 42-46, the CHO cells are ExpiCHO-S™ cells sold under Cat #A29127 by Gibico.
48. The method of any one of clauses 44-47, the medium as used in the method is the ExpiCHO™ Expression Medium sold under Cat #A29100 by Gibico.
49. The method of any one of clauses 44-48, the adapted CHO cells are transfected by using ExpiFectamine™ CHO Transfection Kit (Gibico, Cat #A29129).
50. The method of any one of clauses 42-49, ExpiFectamine™ CHO Enhancer and ExpiCHO™ Feed are added in step c).
51. The method of any one of clauses 42-50, wherein the CSFV E2 protein is further purified.
52. The method of any one of clauses 42-51, wherein the CSFV E2 protein comprises a fusion peptide and the purification of the CSFV E2 protein is done by affinity to a matrix that specifically binds to the fusion peptide.
53. The method of any one of clauses 42-52, wherein the CSFV E2 protein comprises a His-tag as a fusion peptide and the wherein CSFV E2 protein is purified by binding the CSFV E2 protein via the His-tag to Ni-NTA and release the CSFV E2 protein from the Ni-NTA.
54. The method of any one of clauses 42-52, wherein the CSFV E2 protein comprises a FLAG tag as a fusion peptide and the wherein CSFV E2 protein is purified by binding the CSFV E2 protein via the FLAG tag to a monoclonal antibody specifically binds to the FLAG tag and release the CSFV E2 protein from such monoclonal antibody.
55. The method of any one of clauses 42-52, wherein the CSFV E2 protein is linked to a Fc-fragment and the CSFV E2 protein is purified by binding the CSFV E2 protein via the Fc fragment to protein A or protein G and release the CSFV E2 protein from the protein A or protein G.

### EXAMPLES

The subsequent examples further illustrate the invention in an exemplified manner. It is understood that the invention is not limited to any of those examples as described below. A person skilled in the art understands that the performance, results and findings of these examples can be adapted and applied in a broader sense in view of the general description of the invention.

### Materials and Methods

### 1. Cell Culture

The sf9 cell line was cultured in Excell 420 with 5% fetal bovine serum (FBS) and incubated at 27° C. without CO₂.

The sf+ cell line was cultured in Excell 420 and incubated at 27° C., shaker with a speed of 120 rpm.

PK/WRL cell line was cultured with 10% fetal bovine serum (FBS) and incubated at 37° C. with 5% CO₂.

2. Construction of pVI1393-Based Shuttle Plasmids for QZ07-E2 and QZ07-E2-Mutations

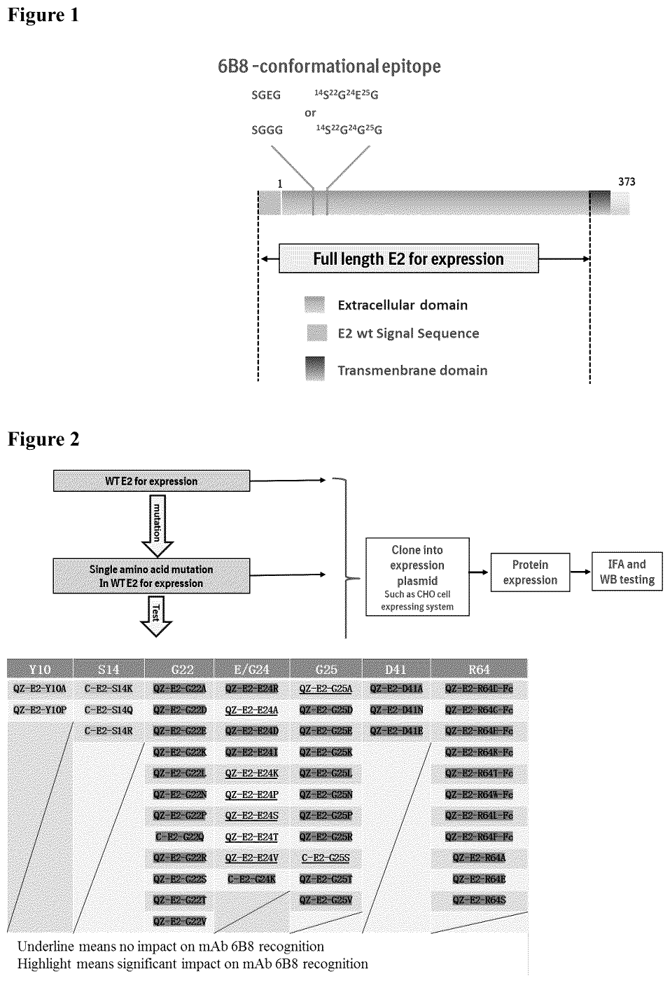

QZ07-E2 sequence, QZ07-E2-KRD and QZ07-E2-KARD sequence were each codon optimized (SEQ ID NOs: 44-46, respectively) and synthesized according the insect expressing expression system. In order to obtain soluble and secret form E2 protein, the last 43 amino acids (aa) of E2 was deleted in final optimized sequence while the last 21 aa from E1 protein was added as signal peptide. Schematic present of E2 structure to be expressed was showed in FIG. 1. Sequences synthesized each were cloned to pVL1393 shuttle plasmids by BamH I and EcoR I to complete the pVL1393-shuttle plasmids for further co-transfection. Whole construction process of CSFV E2 and CSFV E2 with 6B8 epitope mutations refer to FIG. 2. KARD means S14K, G22A, and E24R/G25D mutations, numbering of the amino acid refers to the E2 protein, such as SEQ ID NO:9. Other combinations of mutations, such as KRD (S14K, and E24R/G25D) were also introduced into the E2 protein, respectively.

C-E2 sequence and C-E2-KARD sequence (SEQ ID NOs: 47 and 48, respectively) were each synthesized. In order to obtain soluble and secret form E2 protein, the last 42 amino acids (aa) of E2 was deleted in final sequence while the last 16aa from E1 protein was added as signal peptide. Schematic present of E2 structure to be expressed was the same as showed in FIG. 1. Sequences synthesized each were cloned to pVL1393 shuttle plasmids by BamH I and EcoR I to complete the pVL1393-shuttle plasmids for further co-transfection. Whole construction process of CSFV E2 and CSFV E2 with 6B8 epitope mutations refer to FIG. 2. KARD means S14K, G22A, and G24R/G25D mutations, numbering of the amino acid refers to the E2 protein, such as SEQ ID NO:21.

3. Construction of Recombinant Baculovirus with E2 Expression Cassette,

One well of SF9 cells (1.0×10⁶) was prepared in a six-well plate for transfection and another well was used as cell control. The cells were evenly distributed over the surface after 1 hour incubation. DNA lipoplex transfection mixture was prepared as follows: in one tube, mix 0.5 ml serum-free Grace's insect medium (un-supplemented) and 3 μl DNA shuttle transfection reagent were added; in another tube. 1 μl sapphire baculovirus DNA. 1 μg of transfer plasmid and 0.5 ml serum-free Grace's insect medium (un-supplemented) were added; contents of both tubes were combined into one and mixed gently and placed at room temperature for 20 minutes. Medium was removed from cells and the monolayer was rinsed twice with 1 ml serum free Grace's insect medium (un-supplemented) each time, then medium was removed from cells and DNA/transfection reagent mixture was add. The cell monolayer was incubated for 4-5 hours at 27° C., and transfection mixture was replaced with 2 ml of Excell 420 with 5% FBS. Incubation was continued at 27° C. for 5-6 days. Cells and cell culture medium were collected by centrifuge at 3000 rpm for 10 min at 4° C.

### 4 Plaque Purification Process for Recombinant Baculovirus

Six-well plates with sf9 cells (1.5×10⁶ cells/well) were prepared and leaved at room temperature for 1 hour. 10-fold serial dilutions (50 μL of virus and 450 μL of medium) of each virus, from 10⁻¹ to 10⁻⁶ dilution, were prepared. The cell culture medium was removed from the plates and 100 μL of virus per well from dilutions 10⁻³ to 10⁻⁶ was added in a drop-wise manner to the center of each dish (two wells were infected per dilution). Then the plates were incubated at room temperature for 1 hour. During incubation period. 1% (w/v) LGT agarose medium was prepared at 37° C. water bath. The virus inoculum was removed from each well and 2 ml of 1% (w/v) LGT agarose medium was pipetted and overlay into each well. The plates were incubated at room temperature for about 15 min until solidified. Then 1 ml of insect cell culture medium was added per well on to top of agarose overlay and incubated at 27° C. for 5 days. Finally, liquid overlay was removed and 1 ml of Neutral Red (1:20 with medium) was added to each well, incubated for 2 to 4 hours at 27° C. For the plaques to clear, the dishes were leaved in the dark in the inverted position for 4 hours. The plaques were counted and virus titer was calculated. Individual plaques were pickup with pipette tips and dissolved in 200 μL of medium, stored at 4° C. until propagation.

5 E2 Protein Purification 300 ml culture supernatant was centrifuged and followed by filtration. Filtered supernatant was incubated with Ni sepharose excel beads for 2 hours to capture the target protein. The beads were washed against buffer PBS, pH7.4, and then washed by the buffer containing 20 mM, 50 mM, 80 mM imidazole respectively, finally eluted by the buffer containing 200 mM imidazole and 500 mM imidazole. SDS PAGE and Western blotting were performed to check the purity and concentration of target protein.
6. Construction of Recombinant Plasmid with E2 Expression Cassette for QZ07-E2-Fc-WT and QZ07-E2-Fc-Mutations and Expression in CHO Cells

QZ07-E2-Fc-WT sequence (CSFV E2-Fc fusion protein) and CSFV E2-Fc fusion protein with 6B8 epitope mutations were codon optimized and synthesized according the instruction of CHO expression system (ExpiCHO™ Expression System. Thermo Fisher). In order to obtain soluble and secret form of E2 protein, the last 43 amino acids (aa) of E2 was deleted in final optimized sequence while the last 21 aa from E1 protein was added as signal peptide. A peptide linker was also added between E2 protein and Fc fragment. The amino acid sequence of QZ07-E2-Fc-WT sequence (also named as “QZ07-E2-WT-FL-Fc”) was shown by SEQ ID NO: 97. Sequences synthesized each were cloned to pCDNA3.4 plasmid. Whole construction process of CSFV E2-Fc fusion protein and CSFV E2-Fc fusion protein with 6B8 epitope mutations (such as mutation at position 64) can be referred to FIG. 2.

The transfection and culture was also done by following the instruction of ExpiCHO™ Expression System guide from Thermo Fisher.

On the day prior to transfection (Day-1), the ExpiCHO-S™ culture was splitted to a final density of 3×10⁶-4×10⁶ viable cells/mL, and the cells were allowed to grow overnight. On the day of transfection (Day 0), viable cell density and percent viability were determined. When the cells reached a density of approximately 7×10⁶-10×10⁶ viable cells/mL with a viability of 95-99%, the cells were diluted to a final density of 6×10⁶ viable cells/mL with fresh ExpiCHO™ Expression Medium. The flasks were swirled gently to mix the cells. ExpiFectamine™ CHO/plasmid DNA complexes using cold reagents (4° C.) were prepared. Reagents from refrigeration were simply removed, and DNA complexation was commenced. The following steps comprised: a) gently inverting the ExpiFectamine™ CHO Reagent bottle 4-5 times to mix, b) diluting plasmid DNA with cold OptiPRO™ medium, and mixing by swirling the tube and/or by inversion, c) diluting ExpiFectamine™ CHO Reagent with OptiPRO™ medium, mixing by swirling the tube and/or by inversion or gentel pipetting 2-3 times, mixing by swirling the tube or by inversion, incubating ExpiFectamine™ CHO/plasmid DNA complexes (from Step d) at room temperature for 1-5 minutes, and then slowly transferring the solution to the shaker flask, swirling the flask gently during addition. Day 1. On the day after transfection (Day 1, 18-22 hours post-transfection). ExpiFectamine™ CHO Enhancer and ExpiCHO™ Feed were added to the flask (adding volume according to the guide), followed by gently swirling the flask during addition. The flask was transferred to a 32° C., incubator with a humidified atmosphere of 5% CO₂ in air with shaking and was cultured for 6-10 days. Cell culture medium was collected by centrifuge at 4000 rpm for 15 min at 4° C., and the supernatant was collected.

7. Purification of E2-Fc Fusion Protein and E2-Fc Fusion Protein with 6B8 Epitope Mutations

The purification of E2-Fc fusion protein and E2-Fc fusion protein with 6B8 epitope mutations was performed by using Protein A Agarose from Thermo, respectively. The purification solution comprises 1 M Tris buffer (pH 9.0). The purification process comprises the steps of equilibrating the Protein Agarose and all buffers to room temperature; carefully packing the column with 0.5 ml (for QZ07-E2-Fc-WT and QZ07-E2-Fc mutations, such as mutation at position 64) of resin slurry, following the instructions provided with the columns; equilibrating the column by adding 5 ml of Binding Buffer and allowing the solution to drain through the column; diluting sample at 1:2 with Binding Buffer before application to the Protein A column to maintain the proper ionic strength and pH for optimal binding; applying the diluted sample to the column and allow it to flow completely into the resin; washing the Protein A column with 10 ml of the Binding Buffer; eluting antibodies with 5 ml of Elution Buffer and collect the fraction; immediately adjusting eluted fractions to physiologic pH by adding 100 μl of the Neutralization Buffer 1 ml of elute; transferring the elution into centrifugal filters, and adding 6 ml of PBS; placing the tube at centrifuge. 4000 rpm for 15 min; washing the tube using 10 ml of PBS; repeat the centrifugation step to concentrate the sample into 1-2 ml; using Qubit Kit to measure the purified-buffer exchanged samples; and confirming the product by SDS-PAGE and Western Blot analysis.

### Example 1: Identification and Incorporation of DIVA Sites

A core feature of the desired new vaccine is its ability to differentiate vaccinated animal from infected animal (DIVA). The DIVA feature will be an essential improvement from the traditional CSFV E2 subunit vaccine and has important technical advantage. The strategy of introducing DIVA feature is to alter one or more critical epitope in the immune dominant E2 protein surface and use ELISA to demonstrate the absence of antibody recognizing wild type epitope as an indication of vaccination (negative DIVA).

To implement this strategy, the inventors chose a strongly neutralizing mouse mAb 6B8. Hybridomas producing monoclonal antibody 6B8 was obtained from Zhejiang University and deposited under the accession number CCTCC C2018120 at CCTCC (CHINA CENTER FOR TYPE CULTURE COLLECTION). Wuhan University. Wuhan 430072. P.R. China) on Jun. 13, 2018. Sequencing of the monoclonal antibody 6B8 revealed that it has a heavy chain variable region (VH) having an amino acid sequence as set forth in SEQ ID NO: 7 and a light chain variable region (VL) having an amino acid sequence as set forth in SEQ ID NO: 8. CDRs of this antibody can be easily determined by various methods known in the art, such as Kabat method. For example, mAb 6B8 comprises a VH CDR1 of the amino acid sequence set forth in SEQ ID NO:1, a VH CDR2 of the amino acid sequence set forth in SEQ ID NO:2, a VH CDR3 of the amino acid sequence set forth in SEQ ID NO:3, a VL CDR1 of the amino acid sequence set forth in SEQ ID NO:4, a VL CDR2 of the amino acid sequence set forth in SEQ ID NO:5, and a VL CDR3 of the amino acid sequence set forth in SEQ ID NO:6.

### 1. Characterization of 6B8 mAb

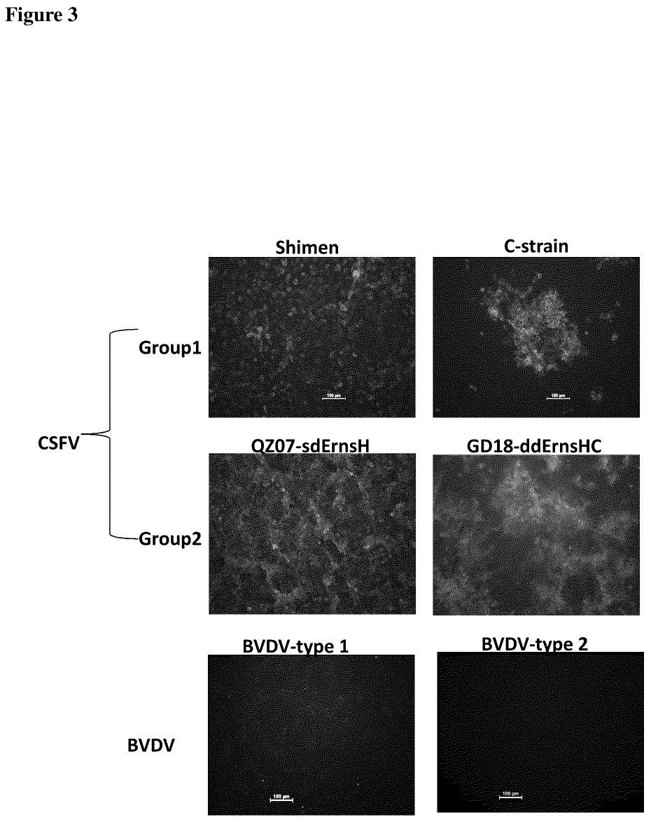

To investigate whether mAb 6B8 can be used for most CSFVs, the inventors tested the binding of mAb 6B8 with various CSF viruses, such as CSFVs from Group 1 (including Shimen strain and C-strain) and from Group2 (including QZ07 and GD18), with two BVDVs as control. The results were shown in FIG. 3. Additional 8 field CSFV isolates from genotype group 2 were also tested as positive for 6B8 mAb (data not shown). These data indicated that 6B8 recognizes a conserved epitope presents on most of CSF viruses, while has no reaction with BVDV viruses.

### 2. Identification of Critical Amino Acids for 6B8 Binding

After serial passage of C-strain virus in PK/WRL cell cultures in the presence of mAb 6B8, escape mutants emerged and can grow in the presence neutralizing concentration of 6B8 antibody. Four clones of such escape mutants were obtained and they all escaped 6B8 binding. Their E2 genes were sequenced and the sequencing results indicated that two nucleotide mutation in two codons (GGAGGT to AGAGAT). These changes translated to two amino acid mutations at consecutive positions 24&25 (Gly-Gly to Arg-Asp, or GG to RD).

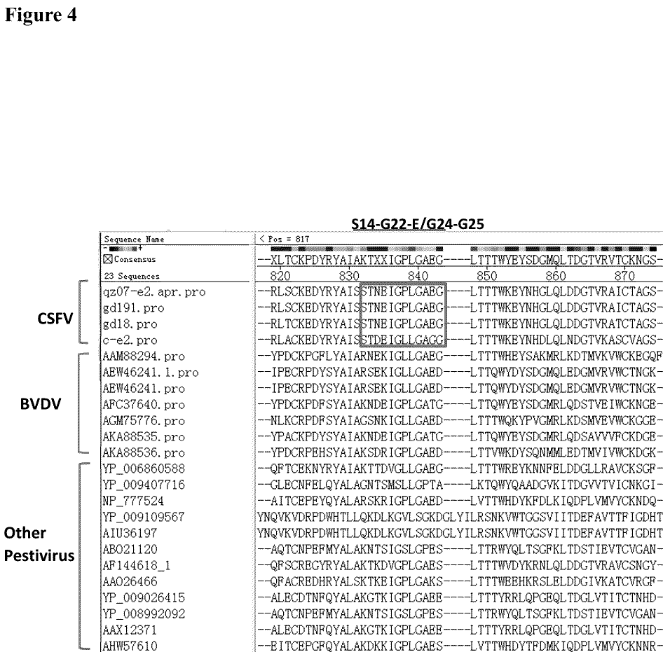

Then, E2 sequence alignment (QZ07, GD18, GD191 and C-strain) was performed with BVDV and other pestivirus E2 to identify other potential critical amino acids for 6B8 binding (FIG. 4). By this approach, additional potential critical amino acids were identified, such as amino acids at position 14 and position 22.

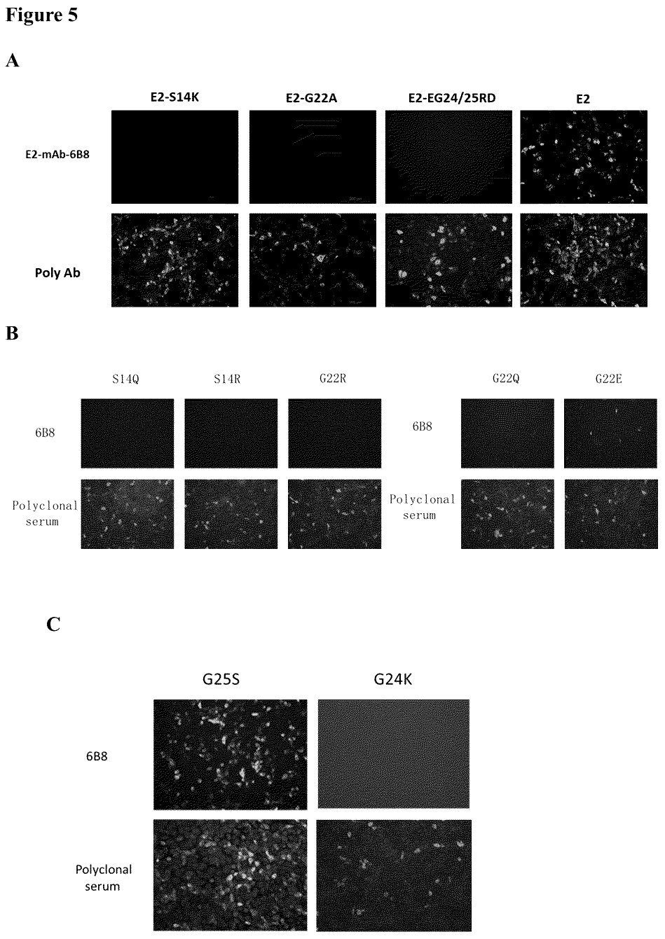

All these potential mutations (S14K, G22A, E24R/G25D) were introduced into E2 expression vector individually to test its effect on 6B8 binding. E2 gene was cloned into pCI-neo-Tag vector (Promega, cat #E1841) to generate expression vectors. After confirmation of the correct expression of E2 protein, all the mutations were introduced into the E2 expression vector. These vectors were then transfected into PK/WRL cells using Lipofectamine3000 (Invitrogen, cat #L3000015) in 24-well plate. 24 hours post transfection, the cells were fixed with 4% formaldehyde and then treated with 0.1% Triton X-100. Cell are then stained with mAb 6B8 or a rabbit-polyclonal antibody against CSFV (used as positive control to detect CSFV with modified 6B8 epitopes), and corresponding Alexa Fluor®488 conjugated second antibody (Invitrogen cat #21206) in an IFA (immunoinfluoscent assay) test. As shown in FIG. 5 A, microscopic examination revealed that S14K, G22A, E24R/G25D mutations are critical for abolishing 6B8 binding.

The inventors also tested the effect of other mutations at positions 14, 22, 24 and 25 individually on the binding with 6B8 antibody. As shown in FIG. 5 B, mutations S14Q, S14R, and G22R totally abolished the binding of 6B8 while G22E, G22Q partially affect the binding of 6B8, further indicating that positions 14 and 22 are critical for 6B8 binding. As shown in FIG. 5 C, a single mutation G24K (for C strain) totally abolished the binding of 6B8, also supporting that position 24 is critical for 6B8 binding. G25S alone cannot abolish the binding of 6B8. However, as the position 25 Gly to Asp mutation emerged together with the mutation at position 24, and thus the two mutations can be considered as one mutation (24/25 mutation).

The results suggest that mutations at position 14, 22, 24 and/or 24/25 may be used for DIVA. The results also suggest that the mutation of 6B8 epitope does not substantially alter the overall immunogenicity of the E2 protein, as the mutated E2 protein can still be recognized by polyclonal antibody against CSFV.

### Example 2: Baculovirus Expression System Construction

Baculovirus expression system of each construct was setup by co-transfection of pV11393-QZ07-E2, QZ07-E2-KARD, QZ07-E2-KRD, C-E2 and C-E2-KARD with baculovirus genome DNA into sf9 cell by commercial kit (Sapphire Baculovirus DNA and transfection Kit: Allele Biotech Cat #ABP-BVD-100029) and recombinant baculovirus containing each E2 expression cassette was purified by plaque purification on Sf9 cell line. The transfected cells were cultured in 6-well plates and incubated at 27° C. for 5 days. Supernatant of each transfected sample was collected and store at 4° C. for further plaque purification.

Plaque purification assay was then conducted for supernatant collected for each constructs as described in above methods. After two rounds of purification, the final recombination baculovirus for with each E2 expression cassette was successfully constructed.

### Example 3: Scale-Up of Expression and Purification of E2 and E2-KARD or E2-KRD

Recombination baculovirus with QZ07-E2, QZ07-E2-KARD, QZ07-E2-KRD, C-E2 and C-E2-KARD expression cassette was amplified by infection of SF+ cell line at MOI 5. 300 ml of supernatant collected from each infected SF+ cell was used for purification as described in method.

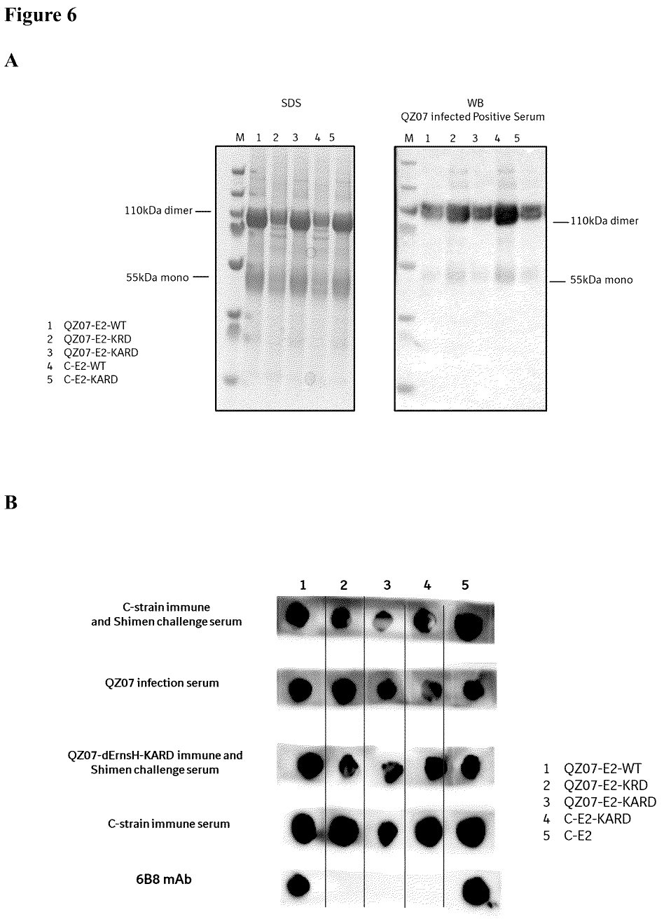

Final products were verified by both SDS PAGE and Western blotting assay. Purified E2 showed correct molecular weight at 110 kDa of dimer-form and 55 kDa of mono-form (FIG. 6A).

Further Dot blot assay showed no reaction of purified QZ07-E2-KARD, QZ07-E2-KRD and C-E2-KARD with 6B8 mAb (FIG. 6B), indicating the each DIVA form of E2 was successfully purified and can be further applied as subunit vaccine. The results also suggest that the mutation of 6B8 epitope does not substantially alter the overall immunogenicity of the E2 protein, as the mutated E2 protein can still be recognized by multiple convalescent swine serum and C-strain vaccinated serum.

### Example 4: Recombinant Plasmid-CHO Expression System Construction and Purification

QZ07-E2-Fc-WT sequence and QZ07-E2-Fc-mutations at position 64 were codon optimized and synthesized according the instruction of CHO expression system (ExpiCHO™ Expression System, Thermo Fisher). In order to obtain soluble and secret form E2 protein, the last 43 amino acids (aa) of E2 was deleted in final optimized sequence while the last 21 aa from E1 protein was added as signal peptide. A peptide linker was also added between E2 protein and Fc fragment. Each synthesized mutation sequence was cloned to pCDNA3.4 plasmid. The transfection and culture for each constructs were then conducted as described in above methods. Finally, the supernatant containing each constructs were successfully collected.

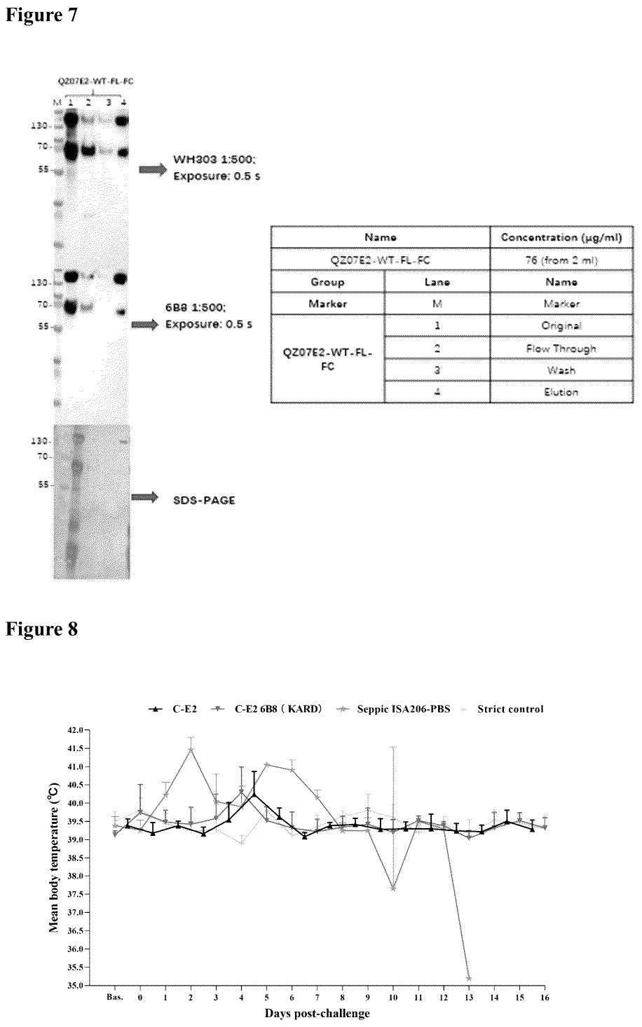

The purification of E2-Fc fusion protein and E2-Fc fusion protein with 6B8 epitope mutation at position 64 were performed by using Protein A Agarose from Thermo, respectively. The purification processes were then conducted as described in above methods. The purification product of E2-Fc fusion protein was confirmed by SDS-PAGE and Western Blot analysis (FIG. 7). The purification product of E2-Fc fusion protein with 6B8 epitope mutation at position 64 was also confirmed by SDS-PAGE and Western Blot analysis, and it shows the similar result for WH303 staining and similar result for SDS-PAGE as the E2-Fc fusion protein, while it shows the negative result for 6B8 staining.

### Example 5: Efficacy Evaluation of E2 and E2-KARD

The objective of this Example was to evaluate the efficacy of the candidate subunit vaccines in 3-week-old piglets.

The two IVPs (Investigational Veterinary Products), adjuvanated C-E2 and C-E2-KARD as expressed in Example 2, are subject to efficacy evaluation.

Briefly, a total of 20 piglets (3-week old) were assigned into 4 groups (Groups 1, 2, 3, and 4), 5 piglet each in Group 1 (C-E2) and Group 2 (C-E2-KARD), were used for IVP test while another 5 piglets in Group 3 served as challenge control. The rest five piglets in Group 4 served as strict (negative) control. On Day 0, animals in groups 1, and 2, were inoculated (IM) with 2 mL Seppic ISA 206 adjuvanated C-E2 (54.2 μg/ml) or C-E2-KARD (55.2 μg/ml) per piglet, respectively. Group 3 was inoculated (IM) with 2 mL PBS+Adjuvant (Seppic ISA 206) on Day 0, served as challenge control. Animals in groups 1, 2, and 3 were inoculated (IM) with CSFV Shimen strain at dose ≥10⁵ MLD/mL on Day 21. All piglets were clinical healthy and free for CSFV and PRRSV antibodies and free of antigen including BVDV. PRV on Day 0. All animals were healthy at the time of immunization.

Rectal temperature and clinical observations were collected daily from D21 to D37. Serum samples were collected every 7 days starting from −Day 7. On Days 21, 24, 28, 31 and 37 (DPC 0, 3, 7, 10, 16), whole blood samples and nasal swap sample of all animals were collected.

### Body Temperature

As shown in FIG. 8, mean body temperature of the challenge control group (Group 3) fluctuated dramatically after challenge, body temperature decreased when pigs moribund. Body temperature of Group 1 and Group 2 rose higher within several days (D2-D4) after challenge but soon fall to similar level of the strict control group.

### Leukocytes Count

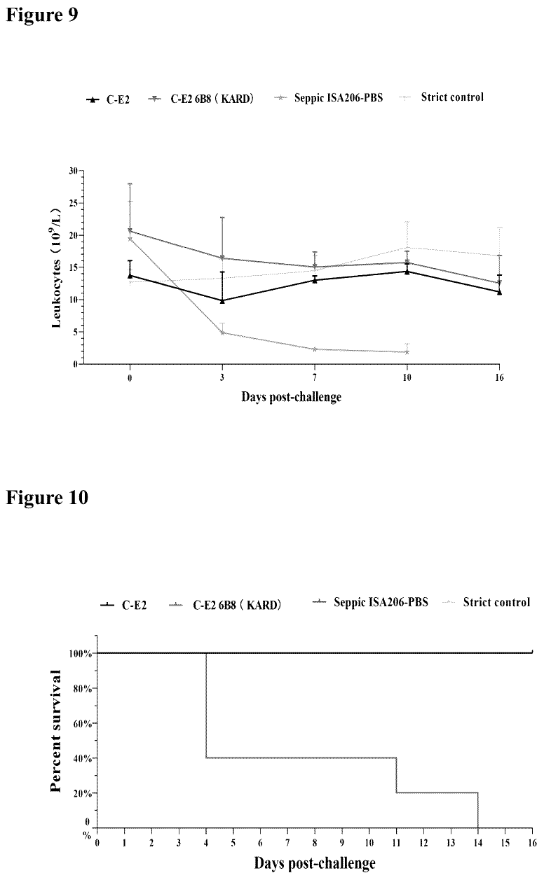

As shown in FIG. 9, Leukocyte counts of the challenge control group decreased dramatically after challenge, while leucocyte counts of animals in the vaccinated groups decreased slightly after challenge and then went up.

### Mortality

As shown in FIG. 10, piglets were all dead in challenge control group (Group 3); no piglet died in other groups.

### Clinical Observation

Clinical observation consist of assessments of liveliness, body tension (stiffness, cramps), body shape (body condition, thinned musculature), breathing, walking, skin, appearance of conjunctiva, appetite and defecation as shown in Table 8. A zero indicates no clinical signs, and increased clinical score indicate an increasing degree of severity of clinical signs. If individual animals show total clinical score above 2 with 3 consecutive observation points is to be considered as CSF related clinical signs.

TABLE 8
Clinical Score Instruction

| No. | Parameters | Criteria | Score |
|---|---|---|---|
| 1 | Liveliness | Attentive (curious , stands up immediately) | 0 |
| | | Slightly reduced ( stands up hesitantly, | 1 |
| | | but without help) | |
| | | Tired, gets up only when forced to, | 2 |
| | | lies down again | |
| | | Dormant, will not stand up | 3 |
| 2 | Body tension | Relaxed, straight back | 0 |
| | | Stiffness and bent back while standing up, | 1 |
| | | afterwards normal | |
| | | Bent back and stiff walking remains | 2 |
| | | Cramps | 3 |
| 3 | Body shape | Full stomach, “round” body | 0 |
| | | Empty stomach | 1 |
| | | Empty stomach, thinned body muscles | 2 |
| | | Emaciated, backbone and ribs visible, head | 3 |
| | | size too big compared to body size | |
| 4 | Breathing | Frequency 10-15/min, barely visible chest | 0 |
| | (judge before | movement | |
| | approaching | Frequency >20/min | 1 |
| | pig) | Frequency >20/min, distinct chest | 2 |
| | | movement | |
| | | Frequency >30/min, breathing through | 3 |
| | | open mouth | |
| 5 | Walking | Well-coordinated movements | 0 |
| | | Hesitant walking, crossed-over legs are | 1 |
| | | corrected slowly | |
| | | Distinct ataxia/hind lameness, able to walk | 2 |
| | | Massive lameness, unable to walk | 3 |
| 6 | Skin (in | Evenly light pink, hair coat flat | 0 |
| | particular | Reddened skin areas | 1 |
| | ears, nose, | Purple-discolored and cold skin areas, | 2 |
| | legs and tail) | few patchier | |
| | | Black-red discoloration of skin, no | 3 |
| | | sensitivity, large hemorrhage in skin | |
| 7 | Eyes/ | Light pink | 0 |
| | conjunctiva | Reddened, clear secretion | 1 |
| | | Highly inflammation, turbid secretion | 2 |
| | | Highly inflammation, purulent secretion, | 3 |
| | | accentuated blood vessels | |
| 8 | Appetite | Greedy, hungry | 0 |
| | | Eats slowly when fed | 1 |
| | | Does not eat when fed, but taste food | 2 |
| | | Does not eat at all, shows no interest | 3 |
| | | for food | |
| 9 | Defecation | Soft feces, normal amount | 0 |
| | | Reduced amount of feces, dry | 1 |
| | | Only small amount of dry, fibrin-covered | 2 |
| | | feces, or diarrhea | |
| | | No feces, mucus in rectum, or watery | 3 |
| | | or bloody diarrhea | |

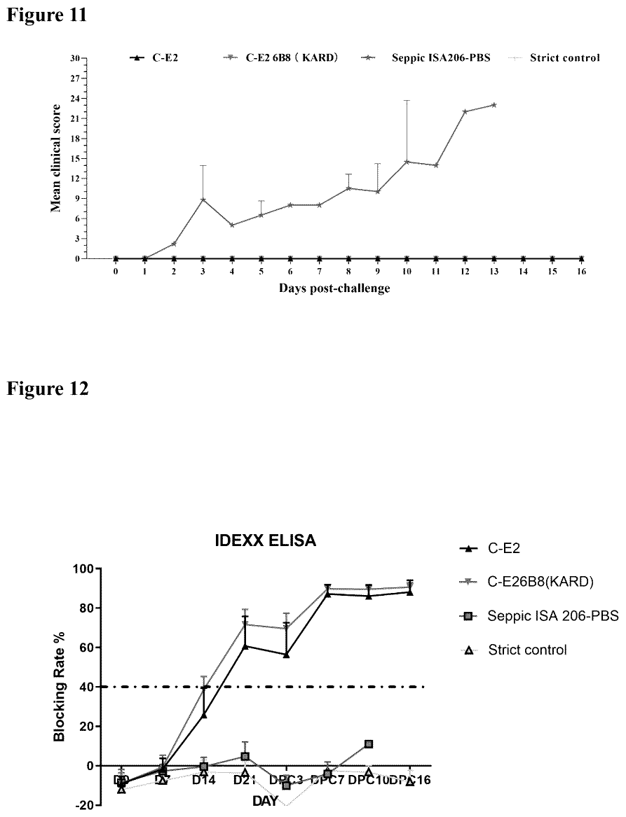

As shown in FIG. 11, mean clinical score of the challenge control group (Group 3) rose higher and higher after challenge; mean clinical score of Group 1, Group 2 and Group 4 were all 0 during the study.

### Virus Isolation

Virus isolation in whole blood, nasal swab and tonsil samples were determined by standard methods in the art. Results are shown in following Table 9. All samples from Group 1 and Group 2 were VI (virus isolation) negative from all collected samples.

TABLE 9

| | Groups | | | | | | | | | | |
|---|---|---|---|---|---|---|---|---|---|---|---|
| Sample | DPC0 | | DPC3 | | DPC7 | | DPC10 | | DPC16 | | |
| Type | WB | NS | WB | NS | WB | NS | WB | NS | WB | NS | Tonsil |
| 1 | 0/5 | 0/5 | 0/5 | 0/5 | 0/5 | 0/5 | 0/5 | 0/5 | 0/5 | 0/5 | 0/5 |
| 2 | 0/5 | 0/5 | 0/5 | 0/5 | 0/5 | 0/5 | 0/5 | 0/5 | 0/5 | 0/5 | 0/5 |
| 3 | 0/5 | 0/5 | 5/5 | 0/5 | 2/2 | 2/2 | 2/2 | 1/2 | 0* | 0 | 5/5 |

WB: whole blood; NS: nasal swab; *piglets of Group 3 were all dead before DPC16.

### Serological Response

The antibody titers of the samples were tested using IDEXX ELISA (Catalog No. 99-43220). As can be seen in FIG. 12, the antibody titers of the two IVP groups were positive (>40%) on D21.

### CONCLUSION

Pigs were protected after vaccinated with the two IVPs, mortality and morbidity rate were all 0%. No viremia or shielding can be detected from IVP groups, and no tonsil tissues were found CSFV positive. Serum on D21 were all positive for the two IVP group. Introduction of the DIVA mutation (in the 6B8 epitope) has no impact on efficacy.

### Example 7: Immunoinfluoscent Assay (IFA) for Determining the Binding of 6B8 mAb to a Mutated 6B8 Epitope

The binding of 6B8 mAb to a mutated 6B8 epitope (test sample) is determined by an immunoinfluoscent assay (IFA) according to the following steps:
1. In a 96-well microtiter plate is seeded with 1.0×10⁶ SF9 cells/well and afterwards infected with the following recombinant baculoviruses at MOI 0.01, each in duplicates:
(i) Test sample: Recombinant baculovirus expressing E2 protein with a modified 6B8 epitope;
(ii) Positive Control: Recombinant baculovirus expressing E2 protein with the wildtype 6B8 epitope;
(iii) Negative Control: Recombinant baculovirus expressing E2 protein with the KARD mutation within the 6B8 epitope as described herein.

The baculovirus infected cells are held in an incubator at about 27° C. for 5 days.
2. The culture media is discarded, and the cells are rinsed once with 1×PBS (200 to 250 μL/well).
3. 100 μl of cold methanol/acetone (50:50) is added per well and incubated at room temperature for 10 min.
4. The fixative is discarded to a defined waste container and plates are dried for 15-30 min under fume hood
5. The 6B8 specific mAb (such as the antibody produced by a hybridoma deposited at CCTCC under the accession number CCTCC C2018120) is diluted with PBS containing 5% BSA to 1:500 to 1:1000, then added to the assay plates with 50 μL/well. The plates are covered with the lid and incubated at 37° C. for 1-2 hour.
6. The assay plates are rinsed 3 times with 1×PBS (250 μL/well).
7. The secondary antibody, Alexa Fluor®488 conjugated Donkey anti-mouse antibody that specifically binds to the 6B8 antibody (Invitrogen, cat #21202), is diluted with PBS containing 5% BSA at 400 fold, added to the assay plates with 50 μL/well. The plates are covered with the lid and incubated at 37° C. for 1 hour.
8. The assay plates are rinsed 3 times with 1×PBS (250 μL/well). At last, 1×PBS is added, 100 μL/well. Final fluorescence signals are read out with an inverted fluorescence microscopy.

A negative result of the Test Sample in this IFA (in both replicates) indicates that the one or more mutations within the 6B8 epitope of the E2 protein leads to a specific inhibition of the binding of a 6B8 monoclonal antibody to such mutated 6B8 epitope.

### Example 8: Dot Blot Assay for Determining the Binding of 6B8 mAb to a Mutated 6B8 Epitope

The binding of 6B8 mAb to a mutated 6B8 epitope (test sample) is determined by a dot blot assay according to the following steps:
1. 1-5 μg of each purified protein diluted in PBS is spotted to NC membrane (Pall, cat #66485), air dried under fume hood for 30 min or longer
(i) Test sample: Recombinant baculovirus expressing E2 protein with a modified 6B8 epitope;
(ii) Positive Control: Recombinant baculovirus expressing E2 protein with the wildtype 6B8 epitope;
2. The membranes are blocked with blocking solution (5% skimmed milk in PBST) at RT for 1 hour.
3. The 6B8 specific mAb (such as the antibody produced by a hybridoma deposited at CCTCC under the accession number CCTCC C2018120) is diluted with PBST containing 5% skimmed milk to 1:800 to 1:1000, then added to each dotted membrane for 10 ml/membrane. The membranes are sealed with the lid and incubated at 37° C. for 1-2 hour.
4. Primary antibody is discarded and each membrane is washed by 3* PBST for 3 times.
5. The secondary antibody, HRP-conjugated anti-mouse antibody (Bio-Rad, STAR117P) that specifically binds to the 6B8 antibody, is diluted with PBST containing 5% skimmed milk at 2000 fold, added to each dotted membrane for 10 ml/membrane. The membranes are sealed with the lid and incubated at 37° C. for 1 hour.
6. Secondary antibody is discarded and each membrane is washed by 3* PBST for 3 times.
7 Blot signal of each membrane is developed with 1-5 mL super signal kit (Thermo, cat #34080) at room temperature.
8. Development time is 1-10 s and the picture is taken with chemdoc (Bio-Rad).

A negative result of the Test Sample in this dot blot indicates that the one or more mutations within the 6B8 epitope of the E2 protein leads to a specific inhibition of the binding of a 6B8 monoclonal antibody to such mutated 6B8 epitope.

### Example 9: Comparison of the Expression of E2 Protein in CHO System and Baculovirus System

QZ07-E2 was codon optimized (using the OptimumGene™ algorithm) (codon optimized sequence of QZ07-E2 is shown in SEQ ID NO:82) and synthesized according the CHO cell expressing expression system (ExpiCHO™ Expression System, Gibico, Cat #A29133). In order to obtain soluble and secret form E2 protein, the last 43 amino acids (aa) of E2 were deleted in final optimized sequence while the last 21 aa from E1 protein was added as the signal peptide. Sequence synthesized was cloned to pcDNA3.4 plasmids.

The ExpiCHO-S™ cells (Gibico, Cat #A29127, which is a clonal derivative of the CHO-S cell line) were adapted to high-density suspension culture in ExpiCHO™ Expression Medium (Gibico, Cat #A2910001). Transfection of the recombinant pcDNA3.4 plasmids was performed according to instruction of ExpiFectamine™ CHO Transfection Kit (Gibico, Cat #A29129). Expression was performed according to User Guide of ExpiCHO™ Expression System (Revision D.0, May 25, 2018, Thermo Fisher Scientific Inc), using ExpiCHO™ Feed (High Titer) method.

Specifically, ExpiCHO-S™ cells were inoculated at a final density of 6×10⁶ viable cell/ml in a flask with fresh ExpiCHO Expression Medium, pre-warmed to 37° C. The flask was swirled gently to mix the cells. Plasmid DNA was diluted with cold OptiPRO Medium and ExpiCHO Fectamine CHO Reagent was diluted with cold OptiPRO medium. Then above two were mixed together and the ExpiFectamine CHO Reagent/plasmid DNA complexes were incubated at Room Temperature for 2 min. Then, the solution was slowly transferred to the shaker flask with swirling the flask during addition. On the day after transfection, ExpiFectamine CHO Enhancer and ExpiCHO Feed were added with gently swirling the flask during addition. The flask was placed to the 32° C., incubator with a humidified atmosphere of 5% CO₂ in air on an orbital shaker, 125±5 rpm. Supernatant and cells were separated by centrifuge and harvested at D4-D10 post transfection.

Expression results of QZ07-E2 in CHO cells in comparison to expression in BEVS (Baculovirus Expression Vector System, see above) are shown in FIG. 13. The yield of E2 in CHO expression system is higher (about 1 mg/ml) compared to BEVS system (about 10 μg/ml).

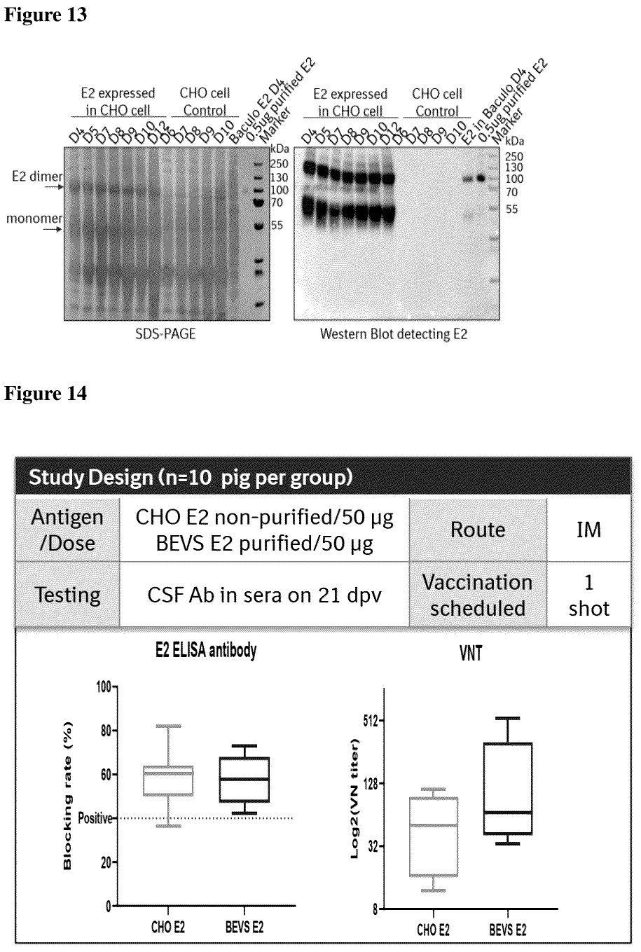

The efficacy of E2 proteins expressed in CHO cells and in BEVS was tested. Specifically, 50 μg non-purified CHO-expressed E2 and 50 μg purified BEVS-expressed E2 were administered (IM) once to pigs (n=10 pigs/per group). CSF antibodies in sera on 21 day-post-vaccination were tested. Results are shown in FIG. 14. It can be seen that CHO expressed E2 maintains its immunogenicity in pigs.

### Example 10: Identification of Additional Critical Residues for 6B8 Binding in E2 Protein

In this Example, amino acid positions 10, 41 and 64 in E2 were identified as additional critical residues for 6B8 mAb binding.

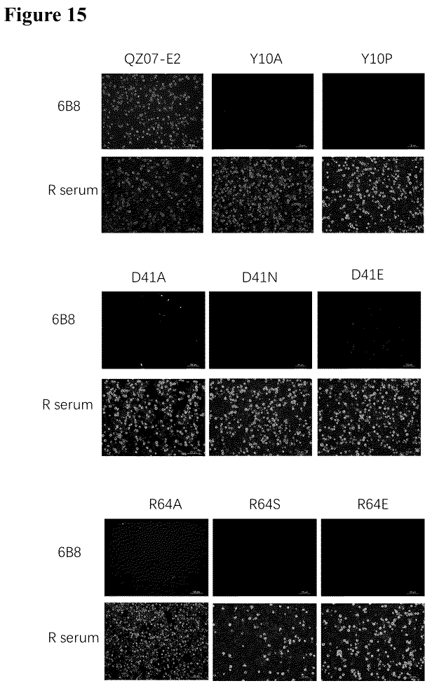

Mutant QZ07-E2 proteins containing Y10A, Y10P. D41A, D41N, D41E, R64A, R64S, or R64E (amino acid sequences shown in SEQ ID NOs: 74-81, respectively; codon-optimized coding sequences shown in SEQ ID NOs: 83-90, respectively) respectively were expressed in CHO cells (according to the method described in previous Example) and subject to IFA (immunoinfluoscent assay) test (according to the method described in previous Example). The results are shown in FIG. 15. Substitutions Y10A, Y10P, D41A. D41N, D41E, R64A, R64S, and R64E can inhibit 6B8 mAb binding respectively, indicating that these mutations may also be used in DIVA.

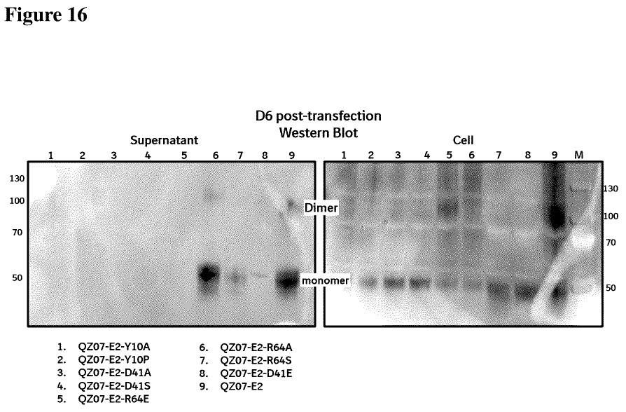

Expression of mutant QZ07-E2 proteins containing Y10A, Y10P, D41A, D41N, D41E, R64A, R64S, or R64E was detected by Western Blotting. The results are shown in FIG. 16. Mutations R64A and R64S only slightly affected the protein expression secretion in CHO cell system. Additional mutation on residue 64 thus will be combined with KARD for expression and efficacy evaluation.

### Example 11: Identification of Additional Critical Amino Acids for Inhibiting 6B8 Binding in E2 Protein

In this Example, more mutations at amino acid positions 22, 24, 25 and 64 in E2 were identified as additional critical for inhibiting 6B8 mAb binding.

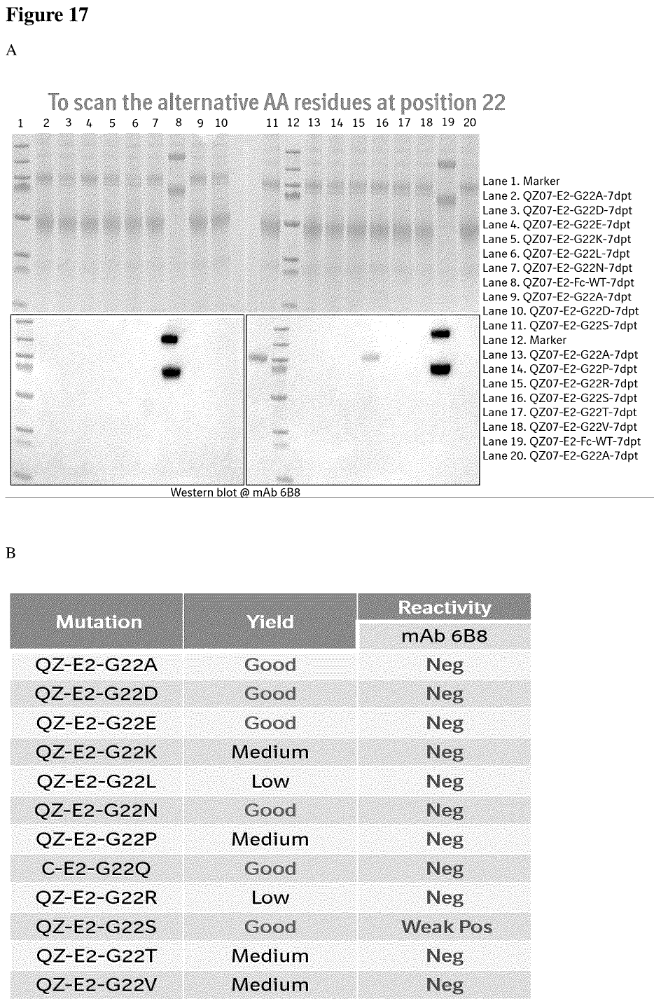

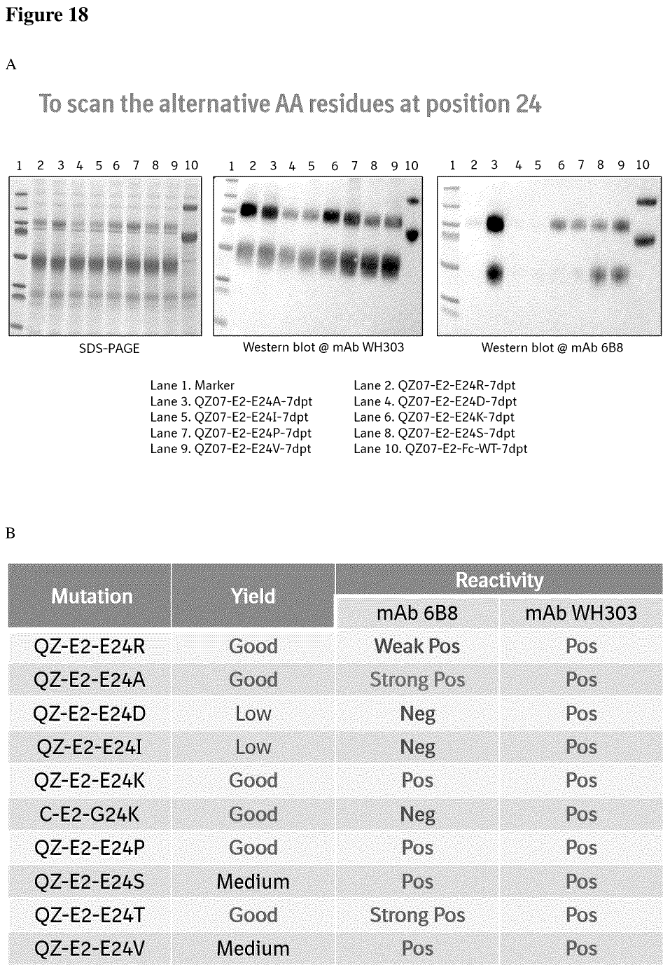

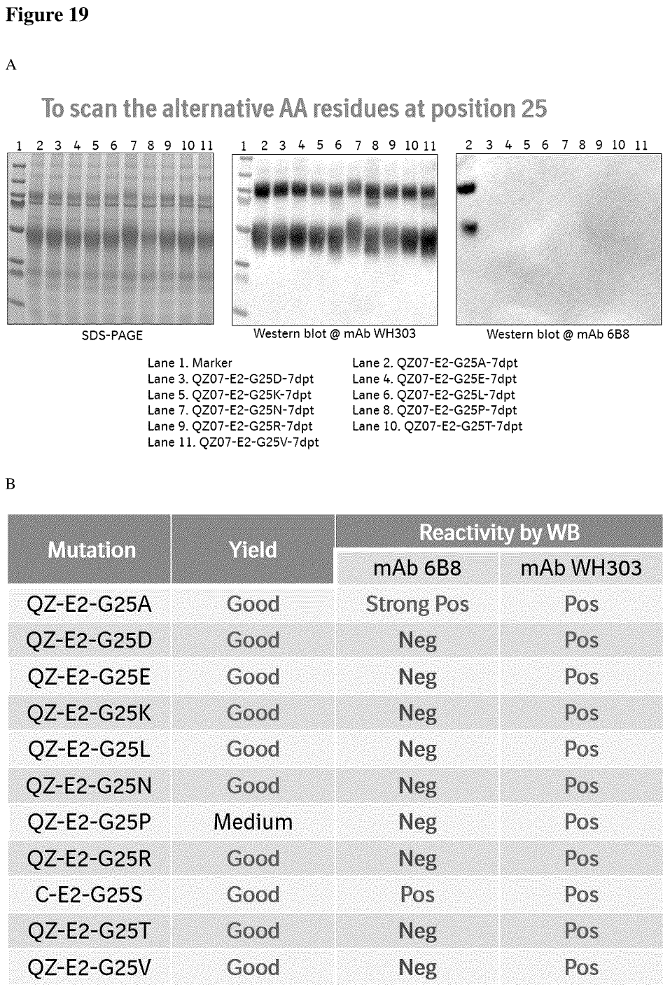

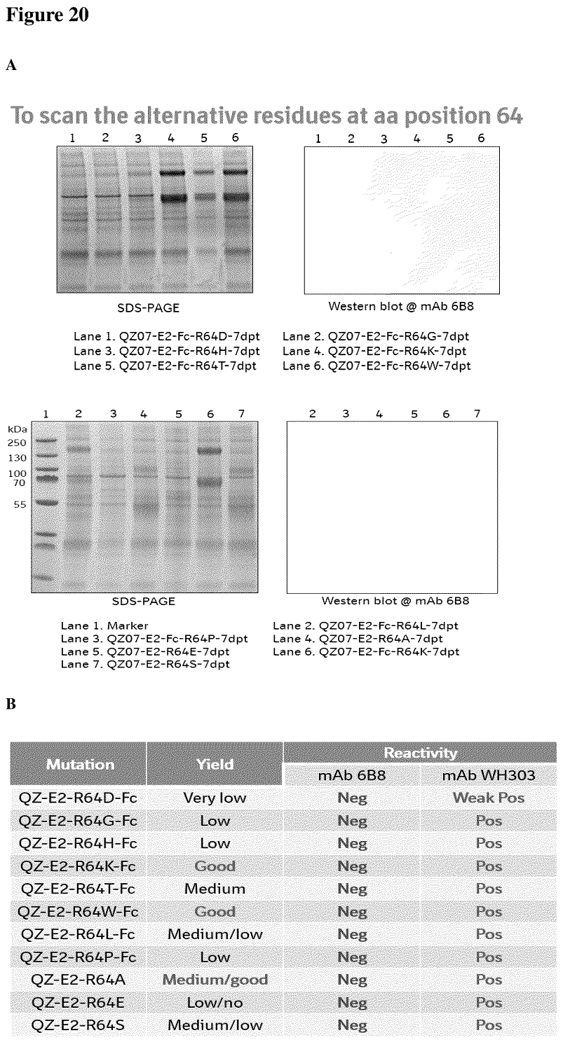

Mutant QZ07-E2 proteins containing various mutations at positions 22, 24, 25 and 64 respectively were expressed in CHO cells (according to the method described in the previous Example) and subject to IFA (immunoinfluoscent assay) test (according to the method described in the previous Example). Expression of mutant QZ07-E2 proteins containing various mutations at positions 22, 24, 25 and 64 respectively was detected by Western Blotting. Some mutants including Mutant QZ07-E2-Fc-R64D, Mutant QZ07-E2-Fc-R64H, Mutant QZ07-E2-Fc-R64T, Mutant QZ07-E2-Fc-R64G, and Mutant QZ07-E2-Fc-R64K were prepared by further linking C terminus of mutant E2 to an Fc fragment for enhancing the protein expression. The method of preparing Mutant QZ07-E2-Fc proteins can be referred to the above examples. The results are shown in FIG. 17-20.

For amino acid position 22, G22A, G22Q, G22D, G22E, G22N and G22S can be candidate substitutions in terms of both yield and reactivity with mAb 6B8.

For amino acid position 24, G24R still have weak reactivity with mAb 6B8. G24D and G24I mutants cannot react with mAb 6B8, although the yield is low.

For amino acid position 25, G25D, G25K, G25L, G25N, G25R, G25T and G25V can be candidate substitutions in terms of both yield and reactivity with mAb 6B8.

For amino acid position 26, R64A, R64K and R64W are the candidate mutants in terms of yield and reactivity with mAb 6B8.

### Example 12: Efficacy Evaluation of Mutant E2 Proteins

E2 proteins with one or more mutations at position 10, 14, 22, 24, 24/25, 41 or 64 are expressed in CHO cells according to the method used in the previous Example. Briefly, codon optimized sequences encoding the mutant E2 proteins are cloned into the pcDNA3.4 vector; the obtained constructs are transfected respectively into ExpiCHO-S™ cells (Gibico, Cat #A29127) which have been adapted to high-density suspension culture in ExpiCHO™ Expression Medium (Gibico, Cat #A2910001); proteins are harvested about 2-14 days post transfection.

Efficacy of the expressed mutant E2 proteins is evaluated according to the method described in the previous Example. Briefly, piglets (3-week old) are assigned into various IVP test groups (according to the mutant E2 protein to be tested), challenge control group and strict (negative) control group. On Day 0, animals in IVP test groups are inoculated (IM) with 2 mL adjuvanated (Seppic ISA 206) mutant E2 proteins (about 50 μg/ml), respectively. Animals in challenge control group are inoculated (IM) with PBS+Adjuvant (Seppic ISA 206) on Day 0. Animals in IVP test groups and challenge control group are inoculated (IM) with CSFV Shimen strain at dose ≥10⁵ MLD/mL on Day 21. Rectal temperature and clinical observations are collected daily from D21 to D37. Serum samples are collected every 7 days starting from −Day 7. On Days 21, 24, 28, 31 and 37 (DPC 0, 3, 7, 10, 16), whole blood samples and nasal swap sample of all animals are collected. Body temperature, leucocyte counts, mortality, clinical scores as listed in Table 8, virus isolation, and serological response are analyzed for evaluation of efficacy.

Expression in CHO cells or introduction of the additional mutation(s) in the 6B8 epitope has no adverse impact on efficacy.

1. A recombinant CSFV (classical swine fever virus) E2 protein comprising at least one mutation within the 6B8 epitope at an amino acid position 10, 41 or 64 of the E2 protein, wherein the at least one mutation within the 6B8 epitope of the E2 protein leads to a specific inhibition of the binding of a 6B8 monoclonal antibody to such mutated 6B8 epitope.

2. (canceled)

3. The recombinant CSFV E2 protein according to claim 1, wherein the 6B8 monoclonal antibody
(i) is produced by a hybridoma deposited at CCTCC under the accession number CCTCC C2018120, or
(ii) comprises a heavy chain variable region (V_{H}) having an amino acid sequence as set forth in SEQ ID NO: 7 and a light chain variable region (V_{L}) having an amino acid sequence as set forth in SEQ ID NO: 8, or
(iii) comprises the CDRs of the monoclonal antibody produced by a hybridoma deposited at CCTCC under the accession number CCTCC C2018120, or
(iv) comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6.

4. The recombinant CSFV E2 protein according to claim 1, wherein the 6B8 epitope of the E2 protein specifically recognized by the 6B8 monoclonal antibody is defined at least by
i) the amino acid residue at position 14, position 22, position 24, positions 24/25, position 10, position 41 and/or position 64 of the E2 protein;
ii) the amino acid residue S14, G22, E24, E24/G25, Y10, D41 and/or R64 of the E2 protein; and/or
iii) the amino acid residue S14, G22, G24, G24/G25, Y10, D41 and/or R64 of the E2 protein.

5. (canceled)

6. The recombinant CSFV E2 protein according to claim 1, which comprises
i) a substitution at amino acid position 24 of the E2 protein, a substitution at amino acid positions 24/25 of the E2 protein, a substitution at amino acid position 14 of the E2 protein, a substitution at amino acid position 22 of the E2 protein, a substitution at amino acid position 10 of the E2 protein, a substitution at amino acid position 41 of the E2 protein, and/or a substitution at amino acid position 64 of the E2 protein;
ii) a substitution at amino acid position 24 of the E2 protein to R, D or I, substitutions at amino acid positions 24 and 25 of the E2 protein to R, D, or I and D, K, L, N, R, T, V, E or P, respectively, a substitution at amino acid position 14 of the E2 protein to K, Q or R, a substitution at amino acid position 22 of the E2 protein to A, R, Q, E, D, N, K, L, P, T, V or S, a substitution at amino acid position 10 of the E2 protein to A or P, a substitution at amino acid position 41 of the E2 protein to A, N or E, and/or a substitution at amino acid position 64 of the E2 protein to A, S, E, D, G, H, T, L, P, K or W; and/or
iii) a substitution of E or G to R, D or I at amino acid position 24 of the E2 protein, substitutions of E or G to R, D or I at amino acid position 24 and G to D, K, L, N, R, T, V, E or P at amino acid position 25 of the E2 protein, a substitution of S to K, Q or R at amino acid position 14 of the E2 protein, a substitution of G to A, R, Q, E, D, N, K, L, P, T, V or S at amino acid position 22 of the E2 protein, a substitution of Y to A or P at amino acid position 10 of the E2 protein, a substitution of D to A, N or E at amino acid position 41 of the E2 protein, and/or a substitution of R to A, S, E, D, G, H, T, L, P, K or W at amino acid position 64 of the E2 protein.

7.-8. (canceled)

9. The recombinant CSFV E2 protein according to claim 1, wherein the recombinant CSFV E2 protein
i) is derived from C-strain or a field strain QZ07 or GD18;
ii) is derived from a field strain QZ07 and comprises a substitution of E to R, D or I at amino acid position 24 of the E2 protein, or substitutions of E to R, D or I at amino acid position 24 and G to D, K, L, N, R, T, V, E or P at amino acid position 25 of the E2 protein, and at least one further substitution selected from the group consisting of a substitution of Y to A or P at amino acid position 10 of the E2 protein, a substitution of D to A, N or E at amino acid position 41 of the E2 protein, and a substitution of R to A, S, E, D, G, H, T, L, P, K or W at amino acid position 64 of the E2 protein;
iii) is derived from a field strain QZ07 and comprises a substitution of E to R, D or I at amino acid position 24 of the E2 protein, or substitutions of E to R, D or I at amino acid position 24 and G to D, K, L, N, R, T, V, E or P at amino acid position 25 of the E2 protein, or a substitution of S to K, Q or R at amino acid position 14 of the E2 protein, or a substitution of G to A, R, Q, E, D, N, K, L, P, T, V or S at amino acid position 22 of the E2 protein, and at least one further substitution selected from the group consisting of a substitution of Y to A or P at amino acid position 10 of the E2 protein, a substitution of D to A, N or E at amino acid position 41 of the E2 protein, and a substitution of R to A, S, E, D, G, H, T, L, P, K or W at amino acid position 64 of the E2 protein;
iv) is derived from a field strain GD18 and comprises a substitution of E to R, D or I at amino acid position 24 of the E2 protein, or substitutions of E to R, D or I at amino acid position 24 and G to D, K, L, N, R, T, V, E or P at amino acid position 25 of the E2 protein;
v) is derived from a field strain GD18, and comprises a substitution of E to R, D or I at amino acid position 24 of the E2 protein, or substitutions of E to R, D or I at amino acid position 24 and G to D, K, L, N, R, T, V, E or P at amino acid position 25 of the E2 protein, and comprises a substitution of S to K, Q or R at amino acid position 14 of the E2 protein or a substitution of G to A, R, Q, E, D, N, K, L, P, T, V or S at amino acid position 22 of the E2 protein, and at least one further substitution selected from the group consisting of a substitution of Y to A or P at amino acid position 10 of the E2 protein, a substitution of D to A, N or E at amino acid position 41 of the E2 protein, and a substitution of R to A, S, E, D, G, H, T, L, P, K or W at amino acid position 64 of the E2 protein;
vi) is derived from C-strain, and comprises a substitution of G to R, D or I at amino acid position 24 of the E2 protein, and a substitution of G to D, K, L, N, R, T, V, E or P at amino acid position 25 of the E2 protein; and/or
vii) is derived from C-strain, and comprises a substitution of G to R, D or I at amino acid position 24 of the E2 protein, and a substitution of G to D, K, L, N, R, T, V, E or P at amino acid position 25 of the E2 protein, and further comprises a substitution of S to K at amino acid position 14 of the E2 protein, a substitution of G to A, R, Q, E, D, N, K, L, P, T, V or S at amino acid position 22 of the E2 protein, a substitution of Y to A or P at amino acid position 10 of the E2 protein, a substitution of D to A, N or E at amino acid position 41 of the E2 protein, and/or a substitution of R to A, S, E, D, G, H, T, L, P, K or W at amino acid position 64 of the E2 protein.

10.-12. (canceled)

13. The recombinant CSFV E2 protein according to claim 1, wherein the recombinant CSFV E2 protein comprises one of the amino acid sequences selected from the group consisting of SEQ ID NOs: 15-20, 22-33, 35-40, 49-72 and 74-81.

14. The recombinant CSFV E2 protein according to claim 1, wherein an Fc fragment is linked to the recombinant CSFV E2 protein.

15. A recombinant nucleic acid coding for the recombinant CSFV E2 protein according to claim 1.

16. A vector comprising the nucleic acid of claim 15.

17. A host cell comprising the nucleic acid of claim 15.

18. A method for producing the recombinant CSFV E2 protein according to claim 1, comprising
(i) culturing a host cell comprising a nucleic acid coding for the recombinant CSFV E2 protein under conditions suitable for the expression of the CSFV E2 protein, and
(ii) isolating and optionally purifying the CSFV E2 protein.

19. A recombinant CSFV (classical swine fever virus) comprising the recombinant E2 protein of claim 1.

20. (canceled)

21. An immunogenic composition comprising the recombinant CSFV E2 protein according to claim 1.

22.-23. (canceled)

24. A method of preventing and/or treating diseases associated with CSFV in an animal, the method comprising the step of administering the immunogenic composition according to claim 21 to an animal in need thereof.

25. A method of differentiating animals infected with CSFV from animals vaccinated with the immunogenic composition of claim 21, comprising
a) obtaining a sample, and
b) testing said sample in an immuno test.

26. The method according to claim 25, wherein the immuno test
i) comprises testing whether an antibody specifically recognizing the 6B8 epitope of the CSFV E2 protein or an antigen-binding fragment thereof can bind to the CSFV E2 protein in the sample;
ii) comprises testing whether an antibody specifically recognizing a 6B8 epitope of the CSFV E2 protein is present in the sample, and/or testing whether an antibody specifically recognizing a mutated 6B8 epitope of the recombinant CSFV E2 protein is present in the sample; and/or
iii) is an EIA (enzyme immunoassay) or ELISA (enzyme linked immunosorbent assay).

27.-28. (canceled)

29. The method according to claim 26, wherein the antibody specifically recognizing the 6B8 epitope
(i) is produced by a hybridoma deposited at CCTCC under the accession number CCTCC C2018120, or
(ii) comprises a heavy chain variable region (V_{H}) having an amino acid sequence as set forth in SEQ ID NO: 7 and a light chain variable region (V_{L}) having an amino acid sequence as set forth in SEQ ID NO: 8, or
(iii) comprises the CDRs of the monoclonal antibody produced by a hybridoma deposited at CCTCC under the accession number CCTCC C2018120, or
(iv) comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6.

30. A kit for differentiating animals infected with CSFV from animals vaccinated with the immunogenic composition of claim 21, which comprises an antibody specifically recognizing the 6B8 epitope of the CSFV E2 protein or an antigen-binding fragment thereof.

31. A method for producing a recombinant CSFV E2 protein comprising the one or more amino acid substitutions as defined in tables 1 to 7, in CHO cells, said method comprises
a) adapting CHO cells to high-density suspension culture in a medium, or growing CHO cells adapted to high-density suspension culture in a medium;
b) transfecting the adapted CHO cells from step a) with a mammalian expression vector comprising the nucleic acid molecule encoding the recombinant CSFV E2 protein;
c) culturing the transfected CHO cells from step b) under conditions suitable for the expression of the recombinant CSFV E2 protein; and
d) harvesting and optionally purifying the recombinant CSFV E2 protein.

32. (canceled)

33. The method of claim 31, wherein in step c), the transfected CHO cells are cultured
i) at about 32-37° C., preferably about 32° C.; and/or
ii) with a humidified atmosphere of about 5-8% CO₂.

33. (canceled)

34. The method of claim 31, wherein the recombinant CSFV E2 protein is harvested about 2-14 days post transfection.


SEQUENCE LISTING (97 sequences)

SEQ ID NO: 1 - protein, 5 aa, Artificial Sequence
  Synthesized
SFGMH

SEQ ID NO: 2 - protein, 17 aa, Artificial Sequence
  Synthesized
YISSDSFTIYYADTMKG

SEQ ID NO: 3 - protein, 7 aa, Artificial Sequence
  Synthesized
GDLPFAY

SEQ ID NO: 4 - protein, 11 aa, Artificial Sequence
  Synthesized
KASQAVGTAVA

SEQ ID NO: 5 - protein, 7 aa, Artificial Sequence
  Synthesized
WASTRHT

SEQ ID NO: 6 - protein, 9 aa, Artificial Sequence
  Synthesized
HQYSSYPLT

SEQ ID NO: 7 - protein, 135 aa, Artificial Sequence
  Synthesized
MDSRLNLVFLVLILKGVQCDVQLVESGGGLVQPGGSRKLSCAASGFTFSSFGMHWVRQAPEKGLEWVAYISSDSFTIYYADTMKGRFTISRDNPQNTLFLQMTSLRSEDTAMYYCARGDLPFAYWGQGTLVTVSA

SEQ ID NO: 8 - protein, 131 aa, Artificial Sequence
  Synthesized
MGIKMETHSQVFVYMLLWLSGVEGDIVMTQSHKFMSTSVGDRVSITCKASQAVGTAVAWYQQKPGQSPKLLIYWASTRHTGVPDRFTGSGSGTDFTLTISNVQSEDLADYFCHQYSSYPLTFGAGTKLELK

SEQ ID NO: 9 - protein, 373 aa, Artificial Sequence
  Synthesized
RLSCKEDYRYAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 10 - protein, 373 aa, Artificial Sequence
  Synthesized
RLTCKEDYRYAISSTNEIGPLGAEGLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 11 - protein, 3898 aa, Artificial Sequence
  Synthesized
MELNHFELLYKTNKQKPMGVEEPVYDIAGRPLFGDPSEVHPQSTLKLPHDRGRGNIRTTLKDLPRKGDCRSGNHLGPVSGIYVKPGPVFYQDYMGPVYHRAPLEFFGEAQFCEVTKRIGRVTGSDGKLYHIYVCIDGCILLKLAKRGAPRSLKWTRNFTDCPLWVTSCSDDGTGDSKDKKPDRMNKGKLKIAPKEHEKDSKTKPPDATIVVEGVKYQVKKKGKVKGKNTQDGLYHNKNKPPESRKKLEKALLAWAVIAIVLYQPVAAENITQWNLSDNGTSGIQQAMYLRGVNRSLHGIWPEKICKGVPTHLATDTELTEIRGMMDASERTNYTCCRLQRHEWNKHGWCNWYNIDPWIQLMNRTQANLTEGPPDKECAVTCRYDKNTDVNVVTQARNRPTTLTGCKKGKNFSFAGTVIEGPCNFNVSVEDILYGDHECGSLFQDTALYLLDGMTNTIEKARQGAARVTSWLGRQLRTTGKKLERGSKTWFGAYALSPYCNVTRKIGYIWYTNNCTPACLPKNTKIIGPGKFDTNAEDGKILHEMGGHLSEFLLLSLVVLSDFAPETASTLYLILHYAIPQSRDEPEVCDTNQLNLTVGLRTEDVVPSSVWNIGKYVCVRPDWWPYETKVALLFEEAGQVIKLALRALRDLTRVWNSASTTAFLICLIKILRGQVVQGIIWLLLVTGAQGRLSCKEDYRYAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAGLQLGQGEVVLIGNLITHTDNEVVVYFLLLYLIIRDEPIKKWILLLFHAMTNNPVKTMTVALLMISGVAKGGKTDGGWQRQPETNFDIQLALAVIVVVVMLLAKRDPTTFPLVITVATLRTAKITNGFSTDLAIATVSAALLTWTYISDYYKYKTWLQYLISTVTGIFLIRVLKGIGELDMHAPTLPSHRPLFYILVYLISTAVVTRWNLDVAGLLLQCVPTLLMVFTMWADILTLILVLPTYELAKLYYLKEVKIGTERGWLWKTNYKRVNDIYEVDQAGEGVYLFPSKQKTSAITSTMLPLIKAILISCISNKWQLIYLLYLIFEVSYYLHKKVIDEIAGGTNFVSRLVAALIEVNWAFDNEEVKGLKKFFLLSSRVKELVIKHKVRNEVVARWFGDEEIYGMPKLIGLVKAATLSKNKHCILCTVCEDRDWRGETCPKCGRFGPPVICGMTLADFEEKHYKRIFIREDQSDGPLREERAGYLQYRARGQLFLRNLPVLATKVKMLLVGNLGTEVGDLEHLGWVLRGPAVCKKVTEHEKCATSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNDDSKPTKLMSGIQTVSKSATDLTEMVKKITTMNRGEFRQITLATGAGKTTELPRSVIEEIGRHKRVLVLIPLRAAAESVYQYMRQKHPSIAFNLRIGEMKEGHMATGITYASYGYFCQMPQPKLRAAMVEYSYIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPVAVKNIMARTDHPEPIQLAYNSYETQVPVLFPKIRNGEVTDTYDTYTFINARKLGDDVPPYVYATEDEDLAVELLGLDWPDPGNQGTVEAGRALKQVVGLSTAENALLVALFGYVGYQALSKRHIPVVTDIYSVEDHRLEDTTHLQYAPNAIKTEGKETELKELAQGDVQRCVEAMTNYARGGIQFMKSQALQVRETPTYKETMNTVADYVKKFIEALSDSKEDILKYGLWGVHTALYKSIGARLGHETAFATLAVKWLAFGGESIADHIKQAATDLVVYYIINRPQFPGDTETQQEGRNFVASLLVSALATYTYKSWNYNNLSKIVEPALATLPYAAKALKLFAPTRLESVVILSTAIYKTYLSIRRGKSDGLLGTGVSAAMEIMSQNPVSVGIAVMLGVGAVAAHNAIEASEQKRTLLMKVFVKNFLDQAATDELVKESPEKIIMALFEAVQTVGNPLRLVYHLYGVFYKGWEAKELAQRTAGRNLFTLIMFEAVELLGVDSEGKIRQLSSNYILELLYKFRDSIKSSVREIAISWAPAPFSCDWTPTDDRIGLPHNNYLQMETRCPCGYRMKAVKTCAGELRLLEEGGSFLCRNKFGRGSRNYRVTKYYDDNLSEIKPVIRMEGHVELYYKGATIKLDFNNSKTVLATDKWEVDHSTLVRALKRHTGAGYQGAYMGEKPNHKHLIERDCATITKDKVYFIKMKRGCAFTYDLSLHNLTRLIELVHKNDLEDREIPAVTVTTWLAYTFVNEDIGTIKPVFGEKVTPEKQEEVALQPAVVVDTTDVAVTVVGETSTMTTGETPTAFTSLGSDSKVQQVLKLGVDEGQYPGPSQQRASLLDAIQGVDERPSVLILGSDKATSNRVKTAKNVKIFRSRDPLELREMMRRGKILVIALCKVDTALLKFVDYKGTFLTRETLEALSLGKPKKKNITKTEAQWLLCLENQIEELPDWFAAEEPVFLEANIKRDKYHLVGDIATIKEKAKQLGATDSTKISKEVGAKVYSMKLSNWVIQEENKQGSLAPLFEELLQQCPPGGQNKTTHMVSAYQLAQGNWMPVGCHVFMGTIPARRTKTHPYEAYVKLRELVDEYKMKTLCGGSGLSKHNEWVIRKIKHQGNLRTKHMLNPGKVAEQLLREGHRHNVYNKTIGSVMTATGIRLEKLPVVRAQTDTTNFHQAIRDKIDKEENLQTPGLHKKLMEVFNALKRPDLEASYDAVEWEELEKGINRKGAAGFFEHKNIGEVLDSEKNKVEEIIDSLRKGRSIRYYETAIPKNEKRDVNDDWTAGDFVDEKKPRVIQYPEAKTRLAITKVMYKWVKQKPVVIPGYEGKTPLFQIFDKVKKEWDQFQNPVAVSFDTKAWDTQVTTGDLELIRDIQKFYFKKKWHKFIDTLTMHMSEVPVISADGEVYIRKGQRGSGQPDTSAGNSMLNVLTMVYAFCEATGVPYKSFDRVAKIHVCGDDGFLITERALGEKFSSKGVQILYEAGKPQKITEGDKMKVAYQFDDIEFCSHTPVQVRWSDNTSSYMPGRNTTTILAKMATRLDSSGERGTIAYEKAVAFSFLLMYSWNPLIRRICLLVLSTELQVRPGKSTTYYYEGDPISAYKEVIGHNLFDLKRTSFEKLAKLNLSMSTLGVWTRHTSKRLLQDCVNVGTKEGNWLVNADRLVSSKTGNRYIPGEGHTQQGKHYEELILARKPISNFEGTDRYNLGPIVNVVLRRLRVMMMALIGRGV

SEQ ID NO: 12 - protein, 3898 aa, Artificial Sequence
  Synthesized
MELNHFELLYKTNKQKPIGVEEPVYDIAGRPFFGDPSEVHPQSTLKLPHDRGRGNIRTTLKNLPRKGDCRSGNHLGPVSGIYVKPGPVFYQDYMGPVYHRAPLEFFEEAQLCEVTKRIGRVTGSDGKLYHIYVCIDGCILLKLAKRGTPRSLKWTRNFTDCPLWVTSCSDDGAGGSKDKKPDRMNKGKLKIAPKEHEKDSKTKPPDATIVVEGVKYQVKKKGKVKGKNTQDGLYHNKNKPPESRKKLEKALLAWAVITIVLYQPVAAENITQWNLSDNGTSGIQQAMYLRGVNRSLHGIWPEKICKGVPTHLATDTELTEIRGMMDASERTNYTCCRLQRHEWNKHGWCNWYNIDPWIQLMNRTQANLTEGPPDKECAVTCRYDKNADVNVVTQARNRPTTLTGCKKGKNFSFAGTIIEGPCNFNVSVEDILYGDHECGSLFQDTALYLLDGMTNTIEKARQGAARVTSWLGRQLSTTGKKLERGSKTWFGAYALSPYCNVTRKVGYIWYTNNCTPACLPKNTKIIGPGKFDTNAEDGKILHEMGGHLSEFLLLSLVILSDFAPETASTLYLILHYAIPQSHEEPEGCDTNQLNLTVGLRTEDVVPSSVWNIGKYVCVRPDWWPYETKVALLFEEAGQVIKLALRALRDLTRVWNSASTTAFLICLIKILRGQVVQGIIWLLLVTGAQGRLTCKEDYRYAISSTNEIGPLGAEGLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAGLQLGQGEVVLIGNLITHMDNEVVVYFLLLYLIIRDEPIKKWILLLFHAMTNNPVKTMTVALLMISGVAKGGKIDGGWQRQPETNFDIQFALAVIIVVVMLLAKRDPTTFPLVITVATLRTAKITSGFSTDLAIATVSATLLTWTYISDYYKYKTWLQYLISTVTGIFLIRVLKGVGELDLHAPTLPSHRPLFYILVYLISTAVVTRWNLDVAGILLQCAPTLLMVFTMWADILTLILILPTYELTKLYYLKEVKIGTERGWLWKTNYKRVNDIYEVDQAGEGVYLFPSKQKTGAITSTVLPLIKAILISCISNKWQFIYLLYLIFEVSYYLHKKIIDEIAGGTNFVSRLVAALIEVNWALDNEEVKGLKKFFLLSSRVKELVIKHKVRNEVMVRWFEDEEIYGMPKLIGLVKAATLSKNKHCILCTVCEDRDWRGETCPKCGRFGPPVICGMTLADFEEKHYKRIFIREDQSDGPLREEHAGYLQYKARGQLFLRNLPVLATKVKMLLVGNLGTEVGDLEHLGWVLRGPAVCKKVTEHEKCATSIMDKLTAFFGVMPRGTTPRAPVRFPTSLLKIRRGLETGWAYTHQGGISSVDHVTCGKDLLVCDTMGRTRVVCQSNNKMTDESEYGVKTDSGCPEGARCYVFNPEAVNISGTKGAMVHLQKTGGEFTCVTASGTPAFFDLKNLKGWSGLPIFEASSGRVVGRVKVGKNEDSKPTKLMSGIQTVSKSATDLTEMVKKITTMNRGEFRQITLATGAGKTTELPRSVIEEIGRHKRVLVLIPLRAAAESVYQYMRQKHPSIAFNLRIGEMEEGYMATGITYASYGYFCQMPQPKLRAAMVEYSYIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPVAVKNIMARTDHPEPIQLAYNSYETQVPVLFPKIRNGEVTDTYDNYTFLNARKLGDDVPPYVYATEDEDLAVELLGLDWPDPGNQGTVEAGRALKQVVGLSTAENALLVALFGYVGYQALSKRHIPVVTDIYSVEDHRLEDTTHLQYAPNAIKTEGKETELKELAQGDVQRCVEAVANYASEGIQFIKSQALKVRETPTYKETMNTVADYVKKFIEALSDSKEDILKYGLWGVHTALYKSIGARLGHETAFATLVVKWLAFGGESVTDHIKQAATDLVVYYIINRPQFPGDTETQQEGRKFVASLLVSALATYTYKSWNYNNLSKIVEPALATLPYAAKALKLFAPTRLESVVILSTAIYKTYLSIRRGKSDGLLGTGVSAAMEIMSQNPVSVGIAVMLGVGAVAAHNAIEASEQKRTLLMKVFVKNFLDQAATDELVKESPEKIIMALFEAVQTVGNPLRLVYHLYGVFYKGWEAKELAQRTAGRNLFTLIMFEAVELLGVDSEGKIRQLSSNYILELLYKFRDDIKSSVREIAISWAPAPFSCDWTPTDDRIGLPHDNYLQMETRCPCGYRMKAVKTCAGELRLLEEGGSFLCRNKFGRGSRNYRVTKYYDDNLSEIKPVIRMEGHVELYHKGATFKLDFDNSKTVLATDRWEVDHSTLIRVLKRHTGAGFQGAYLGEKPNHKHLIERDCATITKDKVCFIKMKRGCAFTYDLSLHNLTRLIELVHKNNLDDREIPAVTVTTWLAYTFVNEDIGTIKPVFGEKVTPEKQEEVALQPAVVVDTTDVAVTVVGETSTMTTGETPTIFTSLGSDSKFQQVLKLGVDEGQYPGPRQQRASLLEAVQGVDERPSVLILGSDKATSNRVKTAKNVKIFRSRDPLELREMMRRGKILIIALCKVDTALLKFVDYKGTFLTRETLEALSLGKPKKRDITKTEARWLLCLEGQLEELPDWFAAKEPIFLEANIKRDKYHLVGDIATIKEKAKQLGATDSTKISKEVGAKVYSMKLSNWVIQEENKQGSLTPLFEELLQQCPPGGQNKTTHMASAYQLAQGNWMPVGCHVFMGTIPARRTKTHPYEAYVKLRELVEEHKMKTSCGGSGLCKHNEWVIRKIKHQGNLRTRHMLNPGKIAEQLDREGHRHNVYNKTIGSVMTATGIRLEKLPVVRAQTDTTNFHQAIRDKIDKEENLQTPGLHKKLMEVFNALKRPELEASYDAVEWEELERGINRKGAAGFFERKNIGEVLDSEKYKVEEIIDSLKKGRSIKYYETAIPKNEKRDVNDDWTAGDFVDEKKPRVIQYPEAKTRLAITKVMYKWVKQKPVVIPGYEGKTPLFQIFDKVKKEWNQFQNPVAVSFDTKAWDTQVTTRDLELIRDIQKFYFKKKWHKFIDTLTMHMSEVPVISADGEVYIRKGQRGSGQPDTSAGNSMLNVLTMVYAFCEATGVPYKSFDRVAKIHVCGDDGFLITERALGEKFASRGVQILYEAGKPQKITEGDNMKVAYQFDDIEFCSHTPVQVRWSDNTSSYMPGRNTTTILAKMATRLDSSGERGTIAYEKAVAFSFLLMYSWNPLIRRICLLVLSTEMQVRPGKSTTYYYEGDPISAYKEVIGHNLFDLKRTSFEKLAKLNLSMSTLGVWTRHTSKRLLQDCVNVGTKEGNWLVNADRLVSSKTGNRYIPGEGHTQQGKHYEELVLARKPTSNFEGTDRYNLGPIVNVVLRRLRVMMMALIGRGV

SEQ ID NO: 13 - DNA, 12296 bp, Artificial Sequence
  Synthesized
gtatacgaggttagttcgttctcgtgtacaatattggacaaaacaaaattccgatttggcctagggcacccctccagcgacggccgaactgggctagccatgcccacagtaggactagcaaacggagggactagccgtagtggcgagctccctgggtggtctaagtcctgagtacaggacagtcgtcagtagttcgacgtgagcaggagcccacctcgagatgctatgtggacgagggcatgcccaagacacaccttaaccctggcgggggtcgccagggtgaaatcacaccatgtgatgggggtacgacctgatagggcgctgcagaggcccactaacaggctagtataaaaatctctgctgtacatggcacatggagttgaatcattttgaactattgtataaaacaaacaaacaaaaaccaatgggagtggaggaaccggtgtatgacatcgcagggagaccattatttggggacccaagtgaggtacacccacaatcaacactgaagctaccacacgacaggggaagaggtaatatcagaactacactgaaggacctacctaggaaaggcgactgcaggagtggaaaccacctaggaccagttagtgggatatacgtaaagcctggccctgtcttctatcaggactacatgggccctgtctaccatagagccccattagagttttttggtgaggcccaattttgtgaggtgaccaagaggataggtagggtgacgggtagtgacggaaagctctaccacatatatgtgtgcatcgacggttgcatactgttgaagttagccaaaaggggcgcgcccagatccctaaagtggactaggaacttcaccgactgtccactgtgggtcactagttgttctgatgatggcacaggtgacagcaaggataaaaagccagacaggatgaacaagggtaaattaaagatagccccaaaagagcatgagaaggacagtaagaccaagccacctgatgctacaattgtggtagagggagtaaaataccaagtaaaaaagaaaggcaaagtcaaagggaagaacactcaagatggcctgtaccataataagaacaaaccaccagagtccaggaagaaactagaaaaagccctattggcctgggcagtgatagcaatagtgctgtaccagcctgtagcagccgagaatataacccaatggaacctgagtgacaacggcacaagcggcatccagcaagctatgtatctcagaggggtcaataggagcttgcatgggatctggcctgaaaaaatatgcaagggggtccctactcatctggccactgacacggaactgacagagatacgcgggatgatggacgccagtgagaggacgaactacacgtgttgtaggttgcagagacacgaatggaacaaacatggctggtgcaactggtacaacatagacccctggattcagttaatgaacaggacccaagcaaatttgacagaaggccctccagataaagagtgtgccgtgacttgcaggtatgacaaaaataccgatgttaacgtggtcacccaggccaggaatagacctactactctgactggctgcaagaaagggaaaaatttttcatttgcaggtacggtcatagagggcccatgcaatttcaacgtatccgtggaggacatcttatatggcgaccatgagtgtggcagcctgttccaggacacggctctgtacctattagatggaatgaccaacactatagagaaagccaggcagggtgcggcaagagtcacatcttggctcgggagacaactcagaaccacagggaagaagttggagagaggaagcaaaacctggttcggtgcctatgccctgtcaccttactgcaatgtaacaaggaaaatagggtacatatggtacacgaacaattgcactccggcatgcctcccaaaaaacacaaaaataataggcccaggaaagtttgacaccaatgcagaagacgggaagattcttcatgaaatggggggccacctatcagaattcttgttgctttctctggtagtcctgtctgacttcgccccagaaacggccagtacattatacctaatcttacactatgcaatccctcagtcccgtgacgaacccgaggtttgcgatacaaaccagctcaacctaacagtgggacttaggacagaagatgtggtaccatcatcagtctggaatattggcaaatatgtgtgtgttagaccagattggtggccgtatgaaactaaggtggctctactgtttgaagaggcgggacaggtcataaagttagctctacgggcactgagggatttaactagagtctggaacagtgcatcaactactgcgttcctcatctgcttgataaaaatattaagaggacaggttgtgcaaggtataatatggctgctgctggtgactggggcacaagggcggttgtcctgcaaggaagactacaggtatgcgatatcatcaaccaatgagatagggccgctaggggctgaaggtctcaccaccacctggaaagaatataaccatggtttgcagctggatgacgggactgtcagggccatttgcactgcagggtcctttaaagttatagcacttaatgtggtcagtaggaggtacctggcatcattacacaagagggctttacccacttcagtaacatttgaactcctatttgatgggactagtccagcaattgaggagatgggagatgactttggatttgggctgtgcccttttgacacaacccccgtggtcaaagggaagtacaataccactttattaaacggtagtgctttctatctagtctgcccaataggatggacgggtgtcatagagtgcacggcagtaagccctacaaccttgagaacagaagtggtgaagaccttcaagagagagaagcctttcccacacagagtgggttgcgtgaccactgttgtagaaaaagaagacctgttctactgcaagtgggggggtaattggacatgtgtaaaaggcaacccggtgacctacatgggggggcaagtaaaacaatgcagatggtgcggttttgacttcaaggagcccgatgggctcccacactaccccataggcaagtgcatcctagcaaatgagacgggttacagggtagtggattccacagactgcaatagagatggcgtcgttatcagcactgaaggtgaacacgagtgcttgattggcaacaccaccgtcaaggtgcacgcgttggatggaagactgggccctatgccgtgcagacccaaagaaatcgtctctagcgcaggacctgtaaggaaaacttcctgcaccttcaactacacaaagacactaagaaacaagtactatgaacccagggacagctatttccagcaatatatgcttaagggcgagtaccaatactggtttgatctggacgtgaccgaccaccacacagactactttgccgaatttgttgtcttggtggtagtggcactattaggggggaggtacgtcctatggctaatagtgacctatgtaattttaacagagcaactcgctgccggtttacagctgggccaaggcgaggtagtactgatagggaacttaattacccacacggacaatgaggtagtggtatacttcttactgctctacctaataataagagacgagcctataaagaaatggatactactgctgtttcatgccatgaccaacaacccagttaagaccatgacagtagcattgctgatgatcagtggggtcgccaagggtgggaaaacagatggtggttggcagaggcagccggagaccaattttgacattcaactcgcactggcagttatagtagtcgttgtgatgttactggcaaagagagacccgactacttttcccctggtgatcacagtggcaaccttaagaacagctaagataaccaatggtttcagcacagatctagctatagccacagtgtcggcagctttgctaacctggacttatatcagtgactactacaaatataagacttggctacagtacctcatcagcacggtgactgggatcttcctaataagggtactgaagggaataggcgagctagacatgcacgccccaactttgccatctcacagaccccttttctacatcctcgtgtacctcatctccactgctgtggtgactagatggaacctggacgtagccggactgctgctacagtgtgtcccaacacttctaatggtgttcacgatgtgggctgacattctcaccctaatcctcgtgttacccacttatgaactggcaaagttgtattaccttaaggaagtgaagatcggaaccgaaagaggatggctgtggaaaactaactataagagggtaaatgacatctatgaagtcgaccaagctggcgagggggtctacctcttcccttcgaaacaaaagactagtgctataacaagcaccatgttgccattgatcaaagccatactcattagctgcatcagtaacaagtggcaactcatatatttactgtacctgatattcgaagtgtcctactaccttcacaagaaagtcatagatgaaatagctggtgggaccaacttcgtttcaaggctagtggcagccttaattgaagttaattgggccttcgacaatgaagaagttaaaggcctaaagaagttctttttgctgtccagtagagttaaagaattggtcatcaaacacaaagtgaggaatgaagtagtggcccgctggttcggagatgaagagatctatgggatgcccaagttgatcggcttagtcaaggcagcaactctaagtaaaaataaacactgtatcttgtgcaccgtctgcgaggacagagattggagaggagaaacctgcccaaaatgtgggcgtttcggaccaccagtgatctgtggtatgaccctagccgacttcgaggagaagcattataagaggatattcatcagggaggaccaatcagatggaccgctcagggaggagcgtgcagggtacttacagtacagagctaggggtcaactgtttctgaggaatctcccagtgttggcgacaaaagtcaagatgctcctggttggtaacctcgggacggaggttggggatttggagcaccttggctgggtgcttagagggcctgctgtctgcaagaaggttactgaacatgaaaagtgtgctacgtctataatggataagttgactgctttcttcggcgttatgccaagaggtaccacccccagagcccccgtgagatttcccacctccctcctaaagataagaagagggctagagacgggttgggcttacacacatcaaggtggcattagctcagttgaccatgtgacttgtgggaaagacttgctggtgtgtgacactatgggccggacaagggtcgtttgccaatcaaataataagatgactgacgaatccgaatacggggtcaaaaccgactcagggtgcccagaaggggccagatgttatgtgttcaaccctgaggcagttaacatatcaggcactaaaggggccatggtccacttacaaaaaactggtggggagttcacctgcgtaacagcatcaggaaccccggccttctttgatctcaagaacctcaagggctggtcagggctaccgatattcgaagcatcgagtggaagggtagtcggaagggtcaaggtcgggaagaacgatgactctaaaccaaccaaactcatgagtgggatacaaacggtctctaaaagtgccactgacctgacggagatggtaaagaagataacaactatgaacaggggagagttcagacagataaccttggccacaggtgctgggaaaactacagagctccccaggtcagtcatagaagagatagggagacacaaaagggtgttggtattgatccccttgagggccgcagcagaatcagtataccaatatatgaggcagaaacacccaagtatagcattcaacctgaggataggggagatgaaggaaggtcacatggccacggggataacctatgcctcctacggttacttttgccagatgccacaacctaagctgagggccgcaatggtagagtactcctacatctttctagatgagtaccattgtgccaccccagaacaactggctatcatggggaagatccacagattctcagagaacctgcgggtggtagctatgacagcgacaccagcagggacagtaacaactactgggcagaaacaccctatagaggagttcatagccccggaagtaatgaaaggagaagacttaggttcagagtacttagacattgccggactaaagataccagtagaggagatgaagaacaacatgctagtttttgtaccaactaggaatatggcggtagaggcggcaaagaaactgaaagctaaaggatacaactcaggctattactacagtggtgaggatccatctaacctgagggtagtcacatcgcagtccccatacgtggttgtagcgaccaacgcgatagaatcaggcgtcactctcccggaccttgacgtggtcgttgacacgggactcaagtgtgagaaaagaatccgattgtcacccaagatgcctttcatagtgactggcctaaaaagaatggctgttaccattggggaacaagcccagagaagagggagggttggaagagtaaagcccgggagatactacaggagccaagaaacccctgtcggctctaaggactaccactacgatttattgcaagcccagaggtacggtatagaagatgggataaatatcaccaaatccttcagagaaatgaactatgattggagcctctatgaggaagatagcctgatgataacacaattggaaatcctcaacaatctactgatatcagaggagctgccggtggcagtaaaaaatataatggctaggactgaccacccagaaccaattcaactagcatataatagctacgagacacaagtgcctgtgttgttcccaaaaataagaaatggagaggtgactgacacttacgatacttacaccttcatcaatgcaagaaaattgggagatgatgtacctccctacgtgtatgccacagaagatgaggacttggcagtggaactgttgggcctagattggccggaccctgggaaccaaggcactgtggaggccggcagagcactaaaacaggtggtcggtctatcaacagccgagaatgccctgttagtagccttgtttggctacgtagggtaccaggccctctcaaagaggcatatacctgtggtcacagacatatactcagttgaagatcacaggctagaggacaccacacacctacagtacgctccgaatgccatcaagacggaggggaaggagactgaattgaaggagttggctcagggggatgtgcagagatgcgtggaagcgatgaccaattacgcgagagggggcatccaatttatgaagtcacaggcactgcaggtgagagaaacccccacttataaagagacaatgaatactgtagcagactatgtgaaaaagtttatcgaggcactgtctgatagcaaggaagacatcttgaaatatgggctgtggggcgtacatacggccttgtacaagagcattggtgccaggcttggtcacgaaaccgcgttcgcaaccctagccgtgaaatggctggcatttgggggggaatcaatagcagatcatataaaacaagcggctacagacttggtcgtctactatatcatcaacagacctcagttcccaggagacacagagacacaacaggaggggagaaattttgtggccagcctactggtctcagccctagcaacttacacatataaaagctggaactataataacctgtccaagatagtagaaccggctttggccaccctgccctatgccgctaaagcccttaaattatttgcccctaccaggctagagagcgtcgtcatattgagcaccgcaatctacaaaacatacctatcaatcaggcgaggcaaaagcgatggattgctaggtacaggggttagtgcggctatggagattatgtcacaaaatccggtatcagtgggcatagcagttatgctgggggttggggctgtagcagcacacaacgcaattgaagccagtgagcagaagagaacactacttatgaaagtttttgtaaagaacttcttggaccaagcagccacagatgaactagtcaaagagagccctgagaaaataataatggccttgtttgaagcagtgcaaacagtaggcaatcctcttaggctagtgtaccacctttacggggttttttataaagggtgggaagcaaaagagttggcccaaaggacagccggcaggaatcttttcaccttgataatgtttgaggccgtggaactattgggagtagatagcgaaggaaaaatccgccaactatcgagtaattacatattagagctcctgtataagttccgtgacagtatcaaatctagtgtgagggagatagcaatcagctgggcccctgccccctttagttgcgattggacaccaacagatgacagaatagggcttccccataacaattacctccaaatggagacaagatgcccttgtggctacaggatgaaagcagtaaaaacctgtgctggggagttgagacttctggaagagggaggttcattcctctgcaggaataaattcggtagagggtcacggaactatagggtgacaaaatactatgatgacaatttatcagaaataaaaccagtgataagaatggaaggacatgtggaactatattataagggggccactatcaaactggactttaacaacagtaaaacagtactggcaaccgacaaatgggaggttgaccattccaccctggtcagggcgctcaagaggcacacaggggctgggtaccaaggagcgtatatgggtgagaaacctaaccacaaacatctgatagagagagactgtgcaacgatcacaaaagacaaggtttacttcatcaagatgaagagggggtgtgcgttcacttatgacttgtccctccacaacctcacccggttaatcgaattggtacacaaaaacgacctggaagatagagaaatccctgctgttacggttacaacctggctggcctacacatttgtgaatgaagacatagggaccataaaaccagtttttggggaaaaagtaacaccggaaaaacaggaggaggtagccttgcagcctgctgtggtggtggacacaacagatgtggccgtgaccgtggtgggggaaacctctactatgactacaggggagaccccaacggcattcaccagcttaggttcagactctaaagttcaacaagttctgaagttaggggtggatgagggtcaataccccgggcccagtcagcagagagcaagcttgctcgacgctatacaaggcgtggatgaaaggccctcggtattgatattgggatctgataaggccacctccaatagggtgaagaccgcaaagaatgtcaagatattcaggagcagagaccccctggaactgagagaaatgatgagaagagggaagatcctggtcatagccctatgtaaggtcgacactgctctactgaaatttgttgattacaaaggcaccttcctgaccagagaaaccctagaggcattaagtttgggtaagcctaagaaaaaaaacataaccaagacagaggcacaatggttgttgtgcctcgaaaaccaaatagaagagctgcctgactggttcgcagctgaggagcccgtatttctggaagccaacatcaaacgtgacaagtatcacctagtgggggatatagctactatcaaagaaaaagctaaacaactgggggcaacagactccacaaagatatcaaaagaggttggagctaaagtatattctatgaagctgagtaactgggtgatacaagaagagaataaacagggcagcttagcccctttgtttgaagagctcctgcaacagtgcccacctgggggccagaataaaaccacacatatggtctcagcctaccaactggctcaagggaactggatgccagttggttgtcacgtgttcatggggaccatacccgccagaagaaccaagacccatccctatgaggcatacgtcaagctgagggagttggtagatgaatataagatgaagacgctatgtggcggttcaggcctaagtaagcacaacgaatgggtaattcgtaagatcaagcatcaagggaacctgaggaccaaacacatgttgaaccccgggaaggttgcagagcaactgcttagagaaggacacagacacaatgtatataataagactataggctcagtgatgacagcaactggtatcaggctggaaaagttacccgtggtcagggcccaaacagacacaaccaacttccatcaagcaataagggataaaatagacaaggaagagaacttacagactccaggcttacacaagaagttaatggaagtcttcaatgcattaaaaagacccgatcttgaggcctcttacgacgctgtggagtgggaggaattggagaaaggaataaataggaagggtgctgcaggtttctttgaacacaagaatataggagaggttctggattcagaaaaaaacaaggttgaagagatcatagacagtttgagaaaaggtaggagtatcaggtactatgaaactgcaatcccaaaaaacgagaagagggacgtcaatgatgactggactgccggtgactttgtagacgagaaaaagcccagggtgatacaataccctgaggctaaaaccaggttggccatcactaaagtaatgtacaagtgggtgaagcagaaaccagtagtcatacccggctatgaaggtaagacacctctgttccaaatctttgacaaagtgaagaaagaatgggaccaattccaaaatccagtggctgtgagctttgacaccaaagcgtgggacacccaggtgactacaggagacctggaactaataagggatatacagaagttctacttcaagaagaagtggcacaaattcattgacaccctaaccatgcacatgtcagaagtacccgtaattagtgccgacggggaggtgtacataaggaaggggcagagaggcagtggacaacctgatacgagcgcaggcaacagcatgttgaatgtattaacaatggtttatgccttctgtgaggccacgggagtgccctataagagttttgacagagtggcaaagatccacgtctgcggggatgatggtttcttgatcactgaaagggctcttggtgaaaaattttcgagtaaaggagtccagatcctatatgaagctggtaagccccaaaaaattacagaaggggataagatgaaagtggcctaccagtttgatgacatcgagttctgctcccatacaccagtgcaagtaaggtggtcagacaatacttccagttatatgccggggaggaacacgactacaatcctggcaaaaatggctacaaggttagattccagtggtgagaggggcactatagcatatgagaaggcggtggcgtttagcttcttattgatgtactcctggaacccactgatcagaaggatatgcttactggtgttgtcaactgaactgcaagtgagaccagggaagtcaaccacctattactatgaaggggacccaatttctgcttacaaggaggtcataggccataatctctttgaccttaaaagaacaagctttgagaaactagctaagttaaatcttagtatgtccacactcggggtatggaccagacacaccagcaaaagattactacaagactgtgtcaacgtcggcactaaagagggcaactggctggttaatgcagacagactggtgagtagcaagacaggaaataggtatatacctggagagggtcatacccagcaagggaaacattatgaagaactgatattggcaaggaagccgatcagcaattttgaagggactgatagatacaatttgggcccaatagtcaacgtagtgttgaggaggctgagagtcatgatgatggctcttataggaaggggggtgtaagcatggccggcccttaaccgggccctatcagtagaaccctgttgtaaataacactaacttattatttatttaaatactattatttatttatttatttatttattgaatgagcaaggattggtacaaactacctcatgttaccacactacactcattttaacagcactttagctggagggaaaatcctgacgtccatagttggactaaggtaatttcctaacggccc

SEQ ID NO: 14 - DNA, 12299 bp, Artificial Sequence
  Synthesized
  misc_feature (12256)..(12256): n is a, c, g, or t
gtatacgaggttagttcgttctcgtgtacaatattggacaagaccaaaattccgatttggcctagggcacccctccagcgacggccgaactgggctagccatgcccacagtaggactagcaaacggagggactagccgtagtggcgagctccctgggtggtctaagtcctgagtacaggacagtcgtcagtagttcgacgtgagcaggagcccacctcgagatgctatgtggacgagggcatgcccaagacacaccttaaccctggcgggggtcgccagggtgaaatcacaccatgtgatgggggtacgacctgatagggtgctgcagaggcccactaacaggctagtataaaaaatctctgctgtacatggcacatggagttgaatcattttgaactactatacaaaacaaataaacaaaaaccaataggagtggaggaaccggtgtatgacatcgcaggaagaccatttttcggggacccaagtgaggtacacccacaatcaacactgaagctgccacatgacagggggagaggtaatatcagaactacactgaagaacctacctaggaaaggcgactgtaggagtggaaaccacctaggaccagtcagtgggatatatgtaaaacccggccctgtcttctatcaggactacatgggccctgtctatcatagagctccattggagttttttgaagaggcccagctgtgtgaggtgactaagagaataggtagggtgacgggtagtgatgggaagctttaccacatatatgtgtgcattgacggttgcatactgctgaagttagccaaaaggggcacgcctagatccctaaagtggactaggaacttcaccgactgtccactatgggtcactagttgttctgatgatggcgcaggtggcagcaaggataagaaaccagacaggatgaacaagggtaaattaaaaatagccccaaaagagcatgagaaggacagcaaaaccaagccacctgatgccacgatcgtggtagagggagtaaaataccaagtgaaaaagaaaggcaaagtcaaagggaagaacactcaagatggcctataccataataagaacaaaccaccagagtccaggaagaaactagaaaaagccctactggcttgggcagtgataacaatcgtactgtaccagcctgtagcggccgagaatataactcagtggaacctgagtgacaatggcacaagcggtatccagcaagccatgtatcttagaggggttaataggagtctacatgggatctggcctgaaaaaatatgcaaaggggtccctactcacctggctactgatacggagctgacagagatacgagggatgatggacgccagcgagaggacaaactatacgtgttgcaggttgcagagacacgaatggaataaacatggatggtgtaactggtacaacatagacccctggattcaattaatgaacaggacccaagcaaatctgacagaaggccccccggataaggagtgcgccgtgacttgcaggtatgacaaaaatgctgacgttaacgtggtcacccaggccaggaatagaccaaccactctgaccggctgcaagaaaggaaaaaacttttcatttgcaggtacgattatagagggcccatgcaatttcaacgtctccgtggaggacatcctatatggggaccatgagtgtggcagcctgttccaggacacagctctgtacctattagatggaatgaccaacactatagagaaagccaggcagggtgcggcaagagttacatcctggcttgggaggcaactcagcaccacagggaagaagttggagagaggaagcaaaacctggttcggcgcctacgccctgtcaccttactgtaacgtaacaaggaaagtggggtacatatggtatacgaacaattgcactccggcatgtctcccaaaaaacacgaaaataataggtccaggaaaattcgataccaacgcggaagatgggaagatacttcatgaaatggggggccacctatcagaatttttgttgctttctttagtaatcctgtctgacttcgccccggagacggccagtacgctatacctaatcttacactatgcaatccctcagtcccatgaagagcctgaaggttgcgatacgaaccagctcaatctaacagtgggacttaggacagaagatgtggtaccatcatcagtttggaatatcggcaaatatgtgtgtgtcagaccagactggtggccgtatgaaactaaggtggctttgctgtttgaggaggcaggacaggttataaaactagctctacgggcactgagagatttaactagagtctggaacagtgcatcaactactgcgttcctcatctgcttgataaaaatattaagaggacaggttgtgcaaggtataatatggctgctgctggtaactggggcacaagggcggttgacctgtaaggaagactacaggtatgcgatatcatcaaccaatgagatagggccgcttggagctgaaggtctcactaccacctggaaagaatacaaccatggtttacagctggatgacgggactgtcagggccacttgcactgcagggtcctttaaagttatagcacttaatgtggttagtaggaggtacctggcatcattacacaagagggctttgcccacctcagtaacatttgaactcctatttgatgggaccagcccagtaattgaggagatgggagacgactttggatttgggctgtgcccttttgacacgatccctgtggtcaaagggaagtataacaccaccttattaaacggcagtgctttctatctagtctgccctataggatggacgggtgttatagagtgcacggcagtaagccccacaaccttgagaacagaagtggtgaagaccttcaagagagagaagcctttccctcacagagtggactgcgtgaccactatagtagaaaaagaagacctgttctattgcaggctggggggtaattggacatgcgtgaaaggtgacccggtgacctacacggggggccaagtaaaacaatgcaggtggtgcggtttcaacttcaaggagcctgatgggctcccacactaccccataggcaagtgcatcctagcaaatgagacgggatacagggtagtggactccacagactgcaacagagacggcgtcgtcatcggcactgaaggagagcatgagtgcttgatcggcaacaccaccgtcaaggtgcacgcgctggacggaagactggcccctatgccttgcagacccaaagaaatcgtatctagtgcgggacctgtaaggaaaacttcctgcacatttaactatacaaagactttgaggaacaagtactatgagcccagggacagctacttccagcaatacatgcttaagggcgagtatcaatactggtttgatctggacgtgacagaccaccacacagactacttcgccgagtttgttgtcttggtggtagtggcactattagggggaaggtacgtcctgtggctaatagtgacctatatagttctaacagagcaactcgctgctggtttacagctaggccagggcgaggtggtgctgatagggaacttaatcacccacatggacaacgaggtagtggtatacttcttactgctttacctaataataagagacgagcccataaagaaatggatactactgctgttccatgccatgaccaacaacccagttaagaccatgacagtagcattgctaatgataagcggggtagccaagggtgggaaaatagatggtggttggcagaggcagccggagaccaattttgacatccaattcgcactggcagtcataatagtcgtcgtgatgttgttggcaaagagagatccgactacttttcccctggtgatcactgtggcaaccttgagaacggctaagataaccagtggtttcagcacagatctagccatagccacagtgtcggcaactttgctaacctggacttatatcagtgactactacaaatacaagacttggctacagtacctcatcagcacggtgactggaatttttctgataagggtactgaagggggtcggcgagttggacctacacgccccgactctgccatcccacagaccccttttctacatccttgtgtacctcatctccactgccgtggtaacgagatggaacttggacgtagccggaatactgctacagtgcgccccaacacttctaatggtattcacgatgtgggctgacatcctcaccctaattctcatactgcccacctatgagttgacaaagttatattaccttaaggaagtgaagatagggactgaaagaggatggctgtggaaaactaattacaagagggtaaatgacatctatgaagtcgaccaagctggcgaaggggtttacctcttcccttcgaaacaaaagactggagctataacgagcaccgtgttgccgttgatcaaagccatactcattagctgcatcagtaacaagtggcaattcatatatttactgtacttgatatttgaggtgtcctactaccttcacaagaaaatcatagatgagatagccggcgggaccaacttcgtttcgagactagtggcggctttaattgaagttaattgggctctagacaatgaagaagtcaaaggcctaaagaagtttttcttgttgtctagtagggtcaaagagttggttatcaaacacaaagtgaggaatgaagtaatggtccgctggtttgaagatgaagagatctacgggatgccaaagctgatcggcctggttaaggcagcaacactgagcaaaaacaaacactgtattttgtgcaccgtctgtgaggacagagattggcggggagaaacttgcccgaaatgcgggcgttttggaccaccagtgatctgtggtatgaccctagctgacttcgaggaaaaacactataagaggattttcatcagggaggaccaatcagacgggccgctcagggaggagcatgcagggtacttgcagtacaaagctaggggtcaattgtttctgaggaatctcccagtgttggcaacaaaagtcaagatgcttctggttggcaacctagggacagaggttggggacctggagcaccttggctgggtgctcagagggcccgctgtctgcaagaaggttactgaacatgaaaagtgtgctacgtctataatggataagttgactgccttctttggtgtcatgccaaggggtaccacccccagagctcctgtaagatttcccacctcccttctaaagataagaagagggctggagacgggttgggcttatacacaccaaggcggtatcagctcagttgaccatgttacttgcgggaaagacctgctagtgtgtgacaccatgggtcggacaagggttgtctgccaatcgaataacaagatgactgacgagtctgaatacggagtcaaaaccgactcagggtgcccagagggagccaggtgctacgtgttcaaccctgaggcagttaatatatcaggcactaaaggagccatggtccacttacagaaaactggtggagaattcacctgtgtaacagcatcagggaccccagctttcttcgatctcaagaacctcaagggttggtcagggctaccgatatttgaagcatcgagtggaagggtagtcggaagggtcaaggtcgggaagaacgaagactctaaaccaaccaaactcatgagtgggatacaaacggtctctaaaagtgccactgatttgacggagatggtgaagaagataacaaccatgaacaggggggagttcagacaaataaccttggccacaggtgctggaaaaactacagagctacccaggtcagtcatagaagagatagggagacacaaaagggtgctggtattgatcccccttagggccgcagcagaatcagtataccaatacatgaggcagaaacacccgagtatagcattcaacctgaggataggggagatggaggaaggatacatggccacgggaataacctatgcttcttatggttacttttgccagatgccacaacccaagttgagagccgcaatggtagagtattcctacatctttctagacgagtaccattgcgccaccccggaacaattggctatcatggggaagatccacagattctcagagaacctgcgggtggtcgctatgacagcgacgccggcaggcacggtaacaactactgggcagaaacaccctatagaggagttcatagccccggaagtgatgaaaggagaagacttaggttcagagtacttagacatcgccggactaaagataccagtagaggagatgaagaacaacatgctagtttttgtaccaactaggaacatggcagtggaggcggcaaagaaattgaaagccaaaggatataactcaggctattactacagtggtgaggacccatccaacctgagggtagttacatcgcagtccccatacgtggtggtagcgaccaacgcaatagaatcaggcgtcactctcccagaccttgatgtggtcgttgacacaggactcaagtgtgagaaaagaatccgactgtcacccaagatgcctttcatagtgactggcctgaaaagaatggccgtcactattggggaacaagcccagagaagagggagggttggaagagtaaagcccgggagatactacaggagccaagaaactccagtcggctctaaggactaccactatgacttgctgcaagcccaaaggtacggtatagaagatgggataaatatcaccaaatccttcagagagatgaactacgattggagcctttatgaggaagatagcctgatgataacacaactggaaatcctcaacaatctgttgatatcagaggagctgccggtagcagtaaaaaatataatggctaggactgaccacccagaaccaattcaactagcgtataatagctacgagacacaggtaccagtgttgttcccaaagataaggaatggagaggtgactgacacttacgacaactacaccttcctcaatgcaagaaaattgggagatgatgtacccccctatgtgtatgccacagaagatgaggacttggcagtggaactgttaggcctagattggccagaccctggaaaccaaggcactgtggaggctggcagagcactaaaacaggtggtcggcctatcaacagccgagaatgccctgctagtggccttgtttggctacgtagggtaccaggccctctcaaaaaggcatatacctgtagtcacagacatatattcagttgaagatcacaggttggaagatacaacacacctacagtatgccccgaacgccatcaagacggaggggaaggagactgaattgaaggagttggctcaaggggatgtgcagagatgtgtggaagcagtggccaattatgcgagtgagggcatccaatttattaagtcacaggcactgaaggtgagagagaccccgacttacaaagagacaatgaatacagtggcagactatgtgaaaaagttcattgaggcactgtcagatagcaaggaagacatcttgaaatatgggctgtggggtgtacacacggccttgtataagagcattggtgccagactaggtcacgaaactgcgttcgcaactttagtcgtgaaatggttggcatttgggggggaatcagtaacagatcacataaagcaagcagccacagacttggttgtttactatattatcaacagacctcaattcccaggggacacggagacacaacaagaagggaggaaatttgtggccagcttactggtctcagccctagcaacctacacatacaaaagctggaactacaacaatctgtccaagatagttgaacctgctttggccactttgccctacgccgccaaagcccttaaattattcgcccctacccgactggagagcgtcgtcatattgagcaccgcaatctacaaaacatatctatcaatcaggcgaggcaaaagtgatggattgcttggtacaggggttagtgcggctatggagattatgtcacaaaatccagtatcagtgggcatagcagtcatgctgggggttggggctgtagcagcccataatgcaattgaagccagtgagcagaagagaacactactaatgaaagtctttgtgaaaaacttcttggaccaagcagccacggatgaactagtcaaagagagccctgagaaaataataatggccctgtttgaagcagtgcagacggtaggcaaccctctcagactggtgtaccacctatatggagttttttataaagggtgggaagcaaaagagttggcccaaaggacagccggcaggaacctcttcaccttaataatgttcgaggctgtggaactactgggagtagatagtgaagggaaaatccgccagctctcaagtaactacatattagagctcctgtacaagttccgtgacgacatcaaatctagtgtgagggagatagcaatcagttgggcccctgcccccttcagctgcgactggacaccaacagatgacagaataggacttccccatgacaattatctccagatggagacaagatgtccttgtggttacaggatgaaagcagtaaaaacctgtgccggggagttgagactcctggaagaggggggttcattcctctgcaggaataaattcggcagagggtcacggaattatagggtgacaaaatattatgatgacaacttatcagaaataaaaccagtgataagaatggaaggacacgtggaactgtatcataaaggggccactttcaaactggactttgacaacagtaaaacagtactggcaacagatagatgggaagtcgaccattccaccctgatcagggtgctcaagaggcacacaggggctggatttcaaggggcgtatctgggtgagaaacccaatcacaagcacctgatagagagagactgtgcaacgatcacaaaagacaaggtctgcttcatcaaaatgaagagggggtgcgcgttcacctatgacttatcactccacaaccttacccggctaatcgaattggtacataagaacaacctggatgacagagaaatccctgccgttacggtcacaacctggctggcctacacattcgtgaatgaagacatagggaccataaaaccagtcttcggggaaaaagtaacaccggagaagcaggaggaggtagccttgcagcctgcagtggtggtggacacaacagatgtggccgtgaccgtggtgggggaaacctccactatgaccacaggggagacccccacaatatttaccagcttaggttcggactctaagtttcaacaagtcctgaaactgggggtggatgagggtcaatatcccgggcccaggcagcagagagcaagtttgctcgaagctgtacaaggtgtagatgagaggccctcggtactaatattggggtctgataaggccacctccaatagggtgaagaccgcaaagaatgtgaagatattcaggagcagagaccccctggaactgagagagatgatgagaagagggaagatcctaatcatagccctgtgtaaggtggacaccgctctgctgaaatttgttgattacaaaggtaccttcctgaccagagagaccctagaggcattaagtctgggcaaacctaagaaaagagacataactaagacagaggcacgatggttgttgtgcctcgaaggtcaattagaagagctgcctgactggtttgcagccaaggagcccatattcttagaagccaacatcaaacgtgacaagtatcatctggtgggagacatagctacaatcaaagaaaaagccaagcaactgggggcaacagactccacaaagatatcaaaagaggttggcgcgaaagtgtattctatgaagttgagtaactgggtgatacaggaagagaataaacagggcagcttaacccccttgtttgaagagctcctgcaacagtgcccgcccgggggccagaataaaaccacacatatggcctcagcctaccaattggctcaggggaactggatgccagttggttgccacgtgttcatggggaccatacccgctagaagaaccaagactcatccctacgaggcatatgtcaagttgagggagttggtggaagaacataagatgaagacatcatgtggcggttcaggcctatgtaagcacaacgaatgggtaattcgtaagatcaagcatcaagggaacctgaggaccagacacatgctgaaccccggaaagattgctgagcaactggatagagaagggcacagacacaatgtgtacaacaagaccataggctcagtaatgacagcgactggcatcaggctggaaaagttacccgtggttagagcccagacagatacaactaatttccaccaagcaataagggataaaatagacaaggaagagaacctacagaccccaggcttacacaagaagttaatggaggtattcaatgcattaaaaagacctgagcttgaggcctcttacgacgccgtggagtgggaggaattggagagaggaataaataggaagggtgctgcagggttcttcgaacgcaagaatataggagaggtcctagattcagaaaaatataaggttgaagagatcatagacagtttgaagaaaggtaggagtattaaatattatgaaactgcaatcccaaagaacgaaaagagggatgtcaatgatgactggactgccggagactttgtggatgagaaaaaacccagggtgatacaatatcctgaggccaaaaccaggttagccatcactaaagtgatgtataagtgggtgaaacagaaaccagtagtcatacccggctatgaaggtaagacacctctgttccaaatttttgacaaagtgaaaaaagaatggaaccaattccaaaatccagtggcagtgagctttgacactaaagcatgggatacccaggtgactacaagagacctggaactaataagggatatacagaagttctatttcaagaagaagtggcacaaattcatcgacaccctaaccatgcacatgtcagaagtacccgtgattagtgccgacggggaggtgtatataaggaaagggcagagaggcagtgggcaacctgatacgagcgcaggcaacagtatgctgaatgtgttaacaatggtttatgccttctgtgaggccacgggggtaccctataaaagtttcgacagagtggcaaagatccacgtctgtggggatgatggcttcttgattactgaaagggctcttggtgaaaaatttgcaagtagaggagtccagatcctatatgaagccgggaagccccaaaaaatcactgaaggggacaatatgaaagtggcctatcagtttgacgacatcgagttctgctcccatacaccagtgcaggtaaggtggtcagacaacacctccagttacatgccggggaggaacacaaccacaatactggcaaaaatggctacaaggttggattccagtggtgagagaggcactatagcatatgagaaggcagtggcattcagtttcttgttgatgtactcctggaacccactgattagaaggatatgcttattggtgttatcaactgaaatgcaagtgagaccagggaagtcaaccacctattactatgaaggagacccaatatctgcttacaaggaggtcatcggccataatctctttgaccttaaaagaacaagtttcgagaagctagctaagctaaatcttagtatgtccacactcggggtatggaccaggcataccagcaaaagattactacaagactgtgtcaatgtcggcaccaaagagggcaactggctggttaacgcagacagactggtgagtagtaagacagggaataggtatatacctggagagggtcatacccaacaaggaaaacattatgaagagctggtattggcaagaaaaccgaccagtaattttgaagggactgatagatataatttgggcccaatagtcaacgtagtgttgaggaggctgagagtcatgatgatggctcttataggaaggggggtgtgagagcggccggctcttgaccgggccctatcagtataaccctgttgtaaataacactaacttattatttatctagacactactatttatttatttatttatttattgaatgagcaaggaatggtacgaactacctcatgttaccacactacactcattttaacagcactttagctgaggaaaaaantcctgacgtccatagttggactaaggtaatttcctaacggccc

SEQ ID NO: 15 - protein, 373 aa, Artificial Sequence
  Synthesized
RLSCKEDYRYAISKTNEIGPLGARDLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 16 - protein, 373 aa, Artificial Sequence
  Synthesized
RLSCKEDYRYAISKTNEIGPLAARDLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 17 - protein, 373 aa, Artificial Sequence
  Synthesized
RLSCKEDYRYAISSTNEIGPLGARDLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 18 - protein, 373 aa, Artificial Sequence
  Synthesized
RLTCKEDYRYAISSTNEIGPLGARDLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 19 - protein, 373 aa, Artificial Sequence
  Synthesized
RLTCKEDYRYAISKTNEIGPLGARDLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 20 - protein, 373 aa, Artificial Sequence
  Synthesized
RLTCKEDYRYAISKTNEIGPLAARDLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 21 - protein, 373 aa, Artificial Sequence
  Synthesized
RLACKEDYRYAISSTDEIGLLGAGGLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG

SEQ ID NO: 22 - protein, 373 aa, Artificial Sequence
  Synthesized
RLACKEDYRYAISSTDEIGLLGARDLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG

SEQ ID NO: 23 - protein, 373 aa, Artificial Sequence
  Synthesized
RLACKEDYRYAISKTDEIGLLGARDLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG

SEQ ID NO: 24 - protein, 373 aa, Artificial Sequence
  Synthesized
RLACKEDYRYAISKTDEIGLLAARDLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG

SEQ ID NO: 25 - protein, 330 aa, Artificial Sequence
  Synthesized
RLSCKEDYRYAISKTNEIGPLGARDLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 26 - protein, 330 aa, Artificial Sequence
  Synthesized
RLSCKEDYRYAISKTNEIGPLAARDLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 27 - protein, 330 aa, Artificial Sequence
  Synthesized
RLSCKEDYRYAISSTNEIGPLGARDLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 28 - protein, 330 aa, Artificial Sequence
  Synthesized
RLTCKEDYRYAISSTNEIGPLGARDLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLD

SEQ ID NO: 29 - protein, 330 aa, Artificial Sequence
  Synthesized
RLTCKEDYRYAISKTNEIGPLGARDLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLD

SEQ ID NO: 30 - protein, 330 aa, Artificial Sequence
  Synthesized
RLTCKEDYRYAISKTNEIGPLAARDLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLD

SEQ ID NO: 31 - protein, 331 aa, Artificial Sequence
  Synthesized
RLACKEDYRYAISSTDEIGLLGARDLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDA

SEQ ID NO: 32 - protein, 331 aa, Artificial Sequence
  Synthesized
RLACKEDYRYAISKTDEIGLLGARDLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDA

SEQ ID NO: 33 - protein, 331 aa, Artificial Sequence
  Synthesized
RLACKEDYRYAISKTDEIGLLAARDLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDA

SEQ ID NO: 34 - protein, 373 aa, Artificial Sequence
  Synthesized
RLSCKEDYRYAISSTNEIGPLGAEGLTTTWKEYNHGLQLDDGTVRAICTAGSFKVTALNVVSRRYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLDVTDHHTDYFAEFIVLVVVALLGGRYVLWLIVTYIVLTEQLAAG

SEQ ID NO: 35 - protein, 373 aa, Artificial Sequence
  Synthesized
RLSCKEDYRYAISSTNEIGPLGARDLTTTWKEYNHGLQLDDGTVRAICTAGSFKVTALNVVSRRYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLDVTDHHTDYFAEFIVLVVVALLGGRYVLWLIVTYIVLTEQLAAG

SEQ ID NO: 36 - protein, 373 aa, Artificial Sequence
  Synthesized
RLSCKEDYRYAISKTNEIGPLGARDLTTTWKEYNHGLQLDDGTVRAICTAGSFKVTALNVVSRRYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLDVTDHHTDYFAEFIVLVVVALLGGRYVLWLIVTYIVLTEQLAAG

SEQ ID NO: 37 - protein, 373 aa, Artificial Sequence
  Synthesized
RLSCKEDYRYAISKTNEIGPLAARDLTTTWKEYNHGLQLDDGTVRAICTAGSFKVTALNVVSRRYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLDVTDHHTDYFAEFIVLVVVALLGGRYVLWLIVTYIVLTEQLAAG

SEQ ID NO: 38 - protein, 330 aa, Artificial Sequence
  Synthesized
RLSCKEDYRYAISSTNEIGPLGARDLTTTWKEYNHGLQLDDGTVRAICTAGSFKVTALNVVSRRYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLD

SEQ ID NO: 39 - protein, 330 aa, Artificial Sequence
  Synthesized
RLSCKEDYRYAISKTNEIGPLGARDLTTTWKEYNHGLQLDDGTVRAICTAGSFKVTALNVVSRRYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLD

SEQ ID NO: 40 - protein, 330 aa, Artificial Sequence
  Synthesized
RLSCKEDYRYAISKTNEIGPLAARDLTTTWKEYNHGLQLDDGTVRAICTAGSFKVTALNVVSRRYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLD

SEQ ID NO: 41 - protein, 16 aa, Artificial Sequence
  Synthesized
IVQGVVWLLLVTGAQG

SEQ ID NO: 42 - protein, 21 aa, Artificial Sequence
  Synthesized
ILRGQVVQGIIWLLLVTGAQG

SEQ ID NO: 43 - protein, 21 aa, Artificial Sequence
  Synthesized
VLRGQVVQGVIWLLLVTGAQG

SEQ ID NO: 44 - DNA, 1095 bp, Artificial Sequence
  Synthesized
atgatcctgcgtggtcaggtggtccagggcatcatctggctcctgctcgtgactggtgctcagggaaggctgtcttgcaaggaagactaccgttacgctatctccagcaccaacgaaatcggacctctcggtgctgagggactgaccactacctggaaggagtacaaccacggactgcaactggacgacggtactgtgcgcgctatctgcaccgccggttccttcaaggtcatcgctctgaacgtggtctcccgccgttacctggctagcctgcacaagcgtgccctgcccacttctgtgaccttcgaactgctgttcgacggtacctcaccagccatcgaggaaatgggcgacgacttcggcttcggactgtgccctttcgacactacccccgtggtcaagggaaagtacaacactaccctgctgaacggttccgctttctacctggtctgccctatcggatggactggtgtgatcgagtgcaccgccgtcagccccactaccctgaggactgaagtggtcaagaccttcaagagggagaagcctttcccccacagagtgggatgcgtcactaccgtggtcgagaaggaagacctgttctactgcaagtggggtggcaactggacttgcgtgaagggcaacccagtcacctacatgggaggtcaagtgaagcagtgcagatggtgcggattcgacttcaaggaacctgacggcctgccccactacccaatcggaaagtgcatcctggctaacgagactggctacagggtggtcgactccaccgactgcaacagagacggtgtggtcatcagcaccgagggcgaacacgagtgcctgatcggaaacactactgtgaaggtccacgctctggacggaaggctgggaccaatgccttgccgtccaaaggaaatcgtgtcttcagccggtcctgtccgcaagacttcttgcaccttcaactacactaagaccctgaggaacaagtactacgaacctagagactcatacttccagcagtacatgctgaagggcgagtaccagtactggttcgacctggacgcgggttcgggtggctcaggtcatcatcatcatcatcac

SEQ ID NO: 45 - DNA, 1095 bp, Artificial Sequence
  Synthesized
atgatcctgcgtggtcaggtggtccagggcatcatctggctcctgctcgtgactggtgctcagggaaggctgtcttgcaaggaagactaccgttacgctatctccaagaccaacgaaatcggacctctcggtgctagagacctgaccactacctggaaggagtacaaccacggactgcaactggacgacggtactgtgcgcgctatctgcaccgccggttccttcaaggtcatcgctctgaacgtggtctcccgccgttacctggctagcctgcacaagcgtgccctgcccacttctgtgaccttcgaactgctgttcgacggtacctcaccagccatcgaggaaatgggcgacgacttcggcttcggactgtgccctttcgacactacccccgtggtcaagggaaagtacaacactaccctgctgaacggttccgctttctacctggtctgccctatcggatggactggtgtgatcgagtgcaccgccgtcagcaaggacaccctgaggactgaagtggtcaagaccttcaagagggagaagcctttcccccacagagtgggatgcgtcactaccgtggtcgagaaggaagacctgttctactgcaagtggggtggcaactggacttgcgtgaagggcaacccagtcacctacatgggaggtcaagtgaagcagtgcagatggtgcggattcgacttcaaggaacctgacggcctgccccactacccaatcggaaagtgcatcctggctaacgagactggctacagggtggtcgactccaccgactgcaacagagacggtgtggtcatcagcaccgagggcgaacacgagtgcctgatcggaaacactactgtgaaggtccacgctctggacggaaggctgggaccaatgccttgccgtccaaaggaaatcgtgtcttcagccggtcctgtccgcaagacttcttgcaccttcaactacactaagaccctgaggaacaagtactacgaacctagagactcatacttccagcagtacatgctgaagggcgagtaccagtactggttcgacctggacgcgggttcgggtggctcaggtcatcatcatcatcatcac

SEQ ID NO: 46 - DNA, 1095 bp, Artificial Sequence
  Synthesized
atgatcctgcgtggtcaggtggtccagggcatcatctggctcctgctcgtgactggtgctcagggaaggctgtcttgcaaggaagactaccgttacgctatctccaagaccaacgaaatcggacctctcgcagctagagacctgaccactacctggaaggagtacaaccacggactgcaactggacgacggtactgtgcgcgctatctgcaccgccggttccttcaaggtcatcgctctgaacgtggtctcccgccgttacctggctagcctgcacaagcgtgccctgcccacttctgtgaccttcgaactgctgttcgacggtacctcaccagccatcgaggaaatgggcgacgacttcggcttcggactgtgccctttcgacactacccccgtggtcaagggaaagtacaacactaccctgctgaacggttccgctttctacctggtctgccctatcggatggactggtgtgatcgagtgcaccgccgtcagcaaggacaccctgaggactgaagtggtcaagaccttcaagagggagaagcctttcccccacagagtgggatgcgtcactaccgtggtcgagaaggaagacctgttctactgcaagtggggtggcaactggacttgcgtgaagggcaacccagtcacctacatgggaggtcaagtgaagcagtgcagatggtgcggattcgacttcaaggaacctgacggcctgccccactacccaatcggaaagtgcatcctggctaacgagactggctacagggtggtcgactccaccgactgcaacagagacggtgtggtcatcagcaccgagggcgaacacgagtgcctgatcggaaacactactgtgaaggtccacgctctggacggaaggctgggaccaatgccttgccgtccaaaggaaatcgtgtcttcagccggtcctgtccgcaagacttcttgcaccttcaactacactaagaccctgaggaacaagtactacgaacctagagactcatacttccagcagtacatgctgaagggcgagtaccagtactggttcgacctggacgcgggttcgggtggctcaggtcatcatcatcatcatcac

SEQ ID NO: 47 - DNA, 1152 bp, Artificial Sequence
  Synthesized
atgatcgtgcaaggtgtggtatggctgttactagtaactggggcacaaggccggctagcctgcaaggaagattacaggtacgcaatatcgtcaaccgatgagatagggctacttggggccggaggtctcaccaccacctggaaggaatacaaccacgatttgcaactgaatgacgggaccgtcaaggccagttgcgtggcaggttcctttaaagtcatagcacttaatgtggtcagtaggagatatttggcgtcattgcataagaaggctttacccacttccgtgacattcgagctcctgttcgacgggaccaacccatcaactgaggaaatgggagatgacttcaggtccgggctgtgcccgtttgatacgagtcccgttgttaagggaaagtacaatacgaccttgttgaacggtagtgctttctatcttgtctgcccaatagggtggacgggtgtcatagagtgcacagcagtgagcccaacaactctgaggacagaagtggtaaagaccttcaggagagacaagccctttccgcacagaatggattgtgtgaccaccacagtggaaaatgaagatttattctattgtaagttggggggcaactggacatgtgtgaaaggcgagccagtggtctacacagggggggtagtaaaacaatgtagatggtgtggcttcgacttcgatgggcctgacggactcccgcattaccccataggtaagtgcattttggcaaatgagacaggttacagaatagtagattcaacggactgtaacagagatggcgttgtaatcagcacagaggggagtcatgagtgcttgatcggtaacacgactgtcaaggtgcatgcatcagatgaaagactgggccctatgccatgcagacctaaagagattgtctctagtgctggtcctgtaaggaaaacctcctgtacattcaactacacaaaaactttgaagaacaggtactatgagcccagggacagctacttccagcaatatatgcttaagggtgagtatcagtactggtttgacctggatgcggaattcggttccggaggctccggtgactacaaagaccatgacggtgattataaagatcatgacatcgattacaaggatgacgatgacaagcatcatcaccatcaccat

SEQ ID NO: 48 - DNA, 1152 bp, Artificial Sequence
  Synthesized
atgatcgtgcaaggtgtggtatggctgttactagtaactggggcacaaggccggctagcctgcaaggaagattacaggtacgcaatatcgaaaaccgatgagatagggctacttgcagccagagatctcaccaccacctggaaggaatacaaccacgatttgcaactgaatgacgggaccgtcaaggccagttgcgtggcaggttcctttaaagtcatagcacttaatgtggtcagtaggagatatttggcgtcattgcataagaaggctttacccacttccgtgacattcgagctcctgttcgacgggaccaacccatcaactgaggaaatgggagatgacttcaggtccgggctgtgcccgtttgatacgagtcccgttgttaagggaaagtacaatacgaccttgttgaacggtagtgctttctatcttgtctgcccaatagggtggacgggtgtcatagagtgcacagcagtgagcccaacaactctgaggacagaagtggtaaagaccttcaggagagacaagccctttccgcacagaatggattgtgtgaccaccacagtggaaaatgaagatttattctattgtaagttggggggcaactggacatgtgtgaaaggcgagccagtggtctacacagggggggtagtaaaacaatgtagatggtgtggcttcgacttcgatgggcctgacggactcccgcattaccccataggtaagtgcattttggcaaatgagacaggttacagaatagtagattcaacggactgtaacagagatggcgttgtaatcagcacagaggggagtcatgagtgcttgatcggtaacacgactgtcaaggtgcatgcatcagatgaaagactgggccctatgccatgcagacctaaagagattgtctctagtgctggtcctgtaaggaaaacctcctgtacattcaactacacaaaaactttgaagaacaggtactatgagcccagggacagctacttccagcaatatatgcttaagggtgagtatcagtactggtttgacctggatgcggaattcggttccggaggctccggtgactacaaagaccatgacggtgattataaagatcatgacatcgattacaaggatgacgatgacaagcatcatcaccatcaccat

SEQ ID NO: 49 - protein, 373 aa, ARTIFICIAL SEQUENCE
  mutated E2 with KARD and A64 for QZ07
RLSCKEDYRYAISKTNEIGPLAARDLTTTWKEYNHGLQLDDGTVRAICTAGSFKVIALNVVSRAYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 50 - protein, 373 aa, ARTIFICIAL SEQUENCE
  mutated E2 with KARD and S64 for QZ07
RLSCKEDYRYAISKTNEIGPLAARDLTTTWKEYNHGLQLDDGTVRAICTAGSFKVIALNVVSRSYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 51 - protein, 373 aa, ARTIFICIAL SEQUENCE
  mutated E2 with KARD and E64 for QZ07
RLSCKEDYRYAISKTNEIGPLAARDLTTTWKEYNHGLQLDDGTVRAICTAGSFKVIALNVVSREYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 52 - protein, 330 aa, ARTIFICIAL SEQUENCE
  truncated and mutated E2 with KARD and A64 for QZ07
RLSCKEDYRYAISKTNEIGPLAARDLTTTWKEYNHGLQLDDGTVRAICTAGSFKVIALNVVSRAYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 53 - protein, 330 aa, ARTIFICIAL SEQUENCE
  truncated and mutated E2 with KARD and S64 for QZ07
RLSCKEDYRYAISKTNEIGPLAARDLTTTWKEYNHGLQLDDGTVRAICTAGSFKVIALNVVSRSYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 54 - protein, 330 aa, ARTIFICIAL SEQUENCE
  truncated and mutated E2 with KARD and E64 for QZ07
RLSCKEDYRYAISKTNEIGPLAARDLTTTWKEYNHGLQLDDGTVRAICTAGSFKVIALNVVSREYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 55 - protein, 373 aa, ARTIFICIAL SEQUENCE
  mutated E2 with KARD and A64 for GD18
RLTCKEDYRYAISKTNEIGPLAARDLTTTWKEYNHGLQLDDGTVRATCTAGSFKVIALNVVSRAYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 56 - protein, 373 aa, ARTIFICIAL SEQUENCE
  mutated E2 with KARD and S64 for GD18
RLTCKEDYRYAISKTNEIGPLAARDLTTTWKEYNHGLQLDDGTVRATCTAGSFKVIALNVVSRSYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 57 - protein, 373 aa, ARTIFICIAL SEQUENCE
  mutated E2 with KARD and E64 for GD18
RLTCKEDYRYAISKTNEIGPLAARDLTTTWKEYNHGLQLDDGTVRATCTAGSFKVIALNVVSREYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 58 - protein, 330 aa, ARTIFICIAL SEQUENCE
  truncated and mutated E2 with KARD and A64 for GD18
RLTCKEDYRYAISKTNEIGPLAARDLTTTWKEYNHGLQLDDGTVRATCTAGSFKVIALNVVSRAYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLD

SEQ ID NO: 59 - protein, 330 aa, ARTIFICIAL SEQUENCE
  truncated and mutated E2 with KARD and S64 for GD18
RLTCKEDYRYAISKTNEIGPLAARDLTTTWKEYNHGLQLDDGTVRATCTAGSFKVIALNVVSRSYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLD

SEQ ID NO: 60 - protein, 330 aa, ARTIFICIAL SEQUENCE
  truncated and mutated E2 with KARD and E64 for GD18
RLTCKEDYRYAISKTNEIGPLAARDLTTTWKEYNHGLQLDDGTVRATCTAGSFKVIALNVVSREYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLD

SEQ ID NO: 61 - protein, 373 aa, ARTIFICIAL SEQUENCE
  mutated E2 with KARD and A64 for C-strain
RLACKEDYRYAISKTDEIGLLAARDLTTTWKEYNHDLQLNDGTVKASCVAGSFKVIALNVVSRAYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG

SEQ ID NO: 62 - protein, 373 aa, ARTIFICIAL SEQUENCE
  mutated E2 with KARD and S64 for C-strain
RLACKEDYRYAISKTDEIGLLAARDLTTTWKEYNHDLQLNDGTVKASCVAGSFKVIALNVVSRSYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG

SEQ ID NO: 63 - protein, 373 aa, ARTIFICIAL SEQUENCE
  mutated E2 with KARD and E64 for C-strain
RLACKEDYRYAISKTDEIGLLAARDLTTTWKEYNHDLQLNDGTVKASCVAGSFKVIALNVVSREYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG

SEQ ID NO: 64 - protein, 331 aa, ARTIFICIAL SEQUENCE
  truncated and mutated E2 with KARD and A64 for C-strain
RLACKEDYRYAISKTDEIGLLAARDLTTTWKEYNHDLQLNDGTVKASCVAGSFKVIALNVVSRAYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDA

SEQ ID NO: 65 - protein, 331 aa, ARTIFICIAL SEQUENCE
  truncated and mutated E2 with KARD and S64 for C-strain
RLACKEDYRYAISKTDEIGLLAARDLTTTWKEYNHDLQLNDGTVKASCVAGSFKVIALNVVSRSYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDA

SEQ ID NO: 66 - protein, 331 aa, ARTIFICIAL SEQUENCE
  truncated and mutated E2 with KARD and E64 for C-strain
RLACKEDYRYAISKTDEIGLLAARDLTTTWKEYNHDLQLNDGTVKASCVAGSFKVIALNVVSREYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDA

SEQ ID NO: 67 - protein, 373 aa, ARTIFICIAL SEQUENCE
  mutated E2 with KARD and A64 for GD191
RLSCKEDYRYAISKTNEIGPLAARDLTTTWKEYNHGLQLDDGTVRAICTAGSFKVTALNVVSRAYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLDVTDHHTDYFAEFIVLVVVALLGGRYVLWLIVTYIVLTEQLAAG

SEQ ID NO: 68 - protein, 373 aa, ARTIFICIAL SEQUENCE
  mutated E2 with KARD and S64 for GD191
RLSCKEDYRYAISKTNEIGPLAARDLTTTWKEYNHGLQLDDGTVRAICTAGSFKVTALNVVSRSYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLDVTDHHTDYFAEFIVLVVVALLGGRYVLWLIVTYIVLTEQLAAG

SEQ ID NO: 69 - protein, 373 aa, ARTIFICIAL SEQUENCE
  mutated E2 with KARD and E64 for GD191
RLSCKEDYRYAISKTNEIGPLAARDLTTTWKEYNHGLQLDDGTVRAICTAGSFKVTALNVVSREYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLDVTDHHTDYFAEFIVLVVVALLGGRYVLWLIVTYIVLTEQLAAG

SEQ ID NO: 70 - protein, 330 aa, ARTIFICIAL SEQUENCE
  truncated and mutated E2 with KARD and A64 for GD191
RLSCKEDYRYAISKTNEIGPLAARDLTTTWKEYNHGLQLDDGTVRAICTAGSFKVTALNVVSRAYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLD

SEQ ID NO: 71 - protein, 330 aa, ARTIFICIAL SEQUENCE
  truncated and mutated E2 with KARD and S64 for GD191
RLSCKEDYRYAISKTNEIGPLAARDLTTTWKEYNHGLQLDDGTVRAICTAGSFKVTALNVVSRSYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLD

SEQ ID NO: 72 - protein, 330 aa, ARTIFICIAL SEQUENCE
  truncated and mutated E2 with KARD and E64 for GD191
RLSCKEDYRYAISKTNEIGPLAARDLTTTWKEYNHGLQLDDGTVRAICTAGSFKVTALNVVSREYLASLHKRALPTSVTFELLFDGTTPAVEEMGDDFGFGLCPFDTTPVVKGKYNTTLLNGSAFYLVCPIGWTGVIECTAVSKDTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLD

SEQ ID NO: 73 - protein, 351 aa, ARTIFICIAL SEQUENCE
  truncated QZ07-E2 with signal peptide
ILRGQVVQGIIWLLLVTGAQGRLSCKEDYRYAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 74 - protein, 330 aa, ARTIFICIAL SEQUENCE
  truncated QZ07-E2-Y10P
RLSCKEDYRPAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 75 - protein, 330 aa, ARTIFICIAL SEQUENCE
  truncated QZ07-E2-Y10A
RLSCKEDYRAAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 76 - protein, 330 aa, ARTIFICIAL SEQUENCE
  truncated QZ07-E2-D41A
RLSCKEDYRYAISSTNEIGPLGAEGLTTTWKEYNHGLQLDAGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 77 - protein, 330 aa, ARTIFICIAL SEQUENCE
  truncated QZ07-E2-D41N
RLSCKEDYRYAISSTNEIGPLGAEGLTTTWKEYNHGLQLDNGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 78 - protein, 330 aa, ARTIFICIAL SEQUENCE
  truncated QZ07-E2-D41E
RLSCKEDYRYAISSTNEIGPLGAEGLTTTWKEYNHGLQLDEGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 79 - protein, 330 aa, ARTIFICIAL SEQUENCE
  truncated QZ07-E2-64A
RLSCKEDYRYAISSTNEIGPLGAEGLTTTWKEYNHGLQLDDGTVRAICTAGSFKVIALNVVSRAYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 80 - protein, 330 aa, ARTIFICIAL SEQUENCE
  truncated QZ07-E2-R64S
RLSCKEDYRYAISSTNEIGPLGAEGLTTTWKEYNHGLQLDDGTVRAICTAGSFKVIALNVVSRSYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 81 - protein, 330 aa, ARTIFICIAL SEQUENCE
  truncated QZ07-E2-R64E
RLSCKEDYRYAISSTNEIGPLGAEGLTTTWKEYNHGLQLDDGTVRAICTAGSFKVIALNVVSREYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 82 - DNA, 1053 bp, ARTIFICIAL SEQUENCE
  codon-optimized QZ07-E2 SEQ
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 83 - DNA, 1053 bp, ARTIFICIAL SEQUENCE
  codon-optimized QZ07-E2-Y10P
attctgagggggcaggtcgtccagggcattatttggctgctgctggtcacaggggcacaggggagactgagttgtaaagaggattatcgccccgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 84 - DNA, 1053 bp, ARTIFICIAL SEQUENCE
  codon-optimized QZ07-E2-Y10A
attctgagggggcaggtcgtccagggcattatttggctgctgctggtcacaggggcacaggggagactgagttgtaaagaggattatcgcgccgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 85 - DNA, 1053 bp, ARTIFICIAL SEQUENCE
  codon-optimized QZ07-E2-D41A
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgccggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 86 - DNA, 1053 bp, ARTIFICIAL SEQUENCE
  codon-optimized QZ07-E2-D41N
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacaacggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 87 - DNA, 1053 bp, ARTIFICIAL SEQUENCE
  codon-optimized QZ07-E2-D41E
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgagggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 88 - DNA, 1053 bp, ARTIFICIAL SEQUENCE
  codon-optimized QZ07-E2-R64A
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcgggcctatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 89 - DNA, 1053 bp, ARTIFICIAL SEQUENCE
  codon-optimized QZ07-E2-R64S
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcggtcctatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 90 - DNA, 1053 bp, ARTIFICIAL SEQUENCE
  codon-optimized QZ07-E2-R64E
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcgggagtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 91 - protein, 3 aa, Artificial sequence
  linker
GGS

SEQ ID NO: 92 - protein, 5 aa, Artificial sequence
  linker
GGGGS

SEQ ID NO: 93 - protein, 6 aa, Artificial sequence
  linker
GSGGSG

SEQ ID NO: 94 - protein, 10 aa, Artificial sequence
  linker
GGGGSGGGGS

SEQ ID NO: 95 - protein, 229 aa, Artificial sequence
  Fc amino acid sequence
TKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 96 - DNA, 687 bp, Artificial sequence
  Fc encoding sequence
accaaaactaaaccaccctgccctatctgccctgcctgcgagtctcccggaccttcagtcttcattttcccccctaagcccaaggacaccctgatgatcagcagaacccctcaggtgacatgcgtggtggtggacgtgtcccaggagaaccctgaggtgcagttcagctggtacgtggatggcgtggaggtgcacaccgcccagacaagaccaaaggaggagcagtttaacagcacatatcgcgtggtgtctgtgctgcccatccagcatcaggactggctgaatggcaaggagttcaagtgcaaggtgaacaataaggatctgccagcccccatcaccaggatcatctctaaggctaagggccagacccgggagccacaggtgtacacactgccaccacacgctgaggagctgtcccgcagcaaggtgtccatcacatgtctggtcatcggcttttaccctccagacatcgatgtggagtggcagaggaacggccagcctgagccagagggcaattatcggaccacaccccctcagcaggacgtggatggcacctacttcctgtattctaagttttccgtggataaggcctcttggcagggcggcggcatcttccagtgtgctgtgatgcacgaagcactgcacaaccattacactcagaaaagtatctctaagaccccaggaaaa

SEQ ID NO: 97 - protein, 584 aa, Artificial sequence
  QZ07-E2-Fc-WT with signal peptide
MILRGQVVQGIIWLLLVTGAQGRLSCKEDYRYAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK